SEQUENCE LISTING (4679 sequences)

SEQ ID NO: 1 - protein, 4 aa, synthetic construct
AQAY

SEQ ID NO: 2 - protein, 4 aa, synthetic construct
NAAQ

SEQ ID NO: 3 - protein, 5 aa, synthetic construct
NAAQA

SEQ ID NO: 4 - protein, 22 aa, Adeno-associated virus 5
TNNQSSTTAPATGTYNLQEIVP

SEQ ID NO: 5-136 - skipped (132 entries)

SEQ ID NO: 137 - DNA, 2175 bp, Adeno-associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctctactactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 138 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 139 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 140-743 - skipped (604 entries)

SEQ ID NO: 744 - protein, 15 aa, synthetic construct
ATNNQSSTNAAQAYT

SEQ ID NO: 745 - DNA, 45 bp, synthetic construct
  source 1..45: mol_type = other DNA
gcgacgaataatcagtcttctacgaatgcggcgcaggcgtatacg

SEQ ID NO: 746-942 - skipped (197 entries)

SEQ ID NO: 943 - protein, 6 aa, synthetic construct
NAAQAY

SEQ ID NO: 944 - DNA, 18 bp, synthetic construct
  source 1..18: mol_type = other DNA
aatgcggcgcaggcgtat

SEQ ID NO: 945-981 - skipped (37 entries)

SEQ ID NO: 982 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTNAAQAYTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 983 - skipped

SEQ ID NO: 984 - DNA, 2175 bp, synthetic construct
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggcgacgaataatcagtcttctacgaatgcggcgcaggcgtatacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 985-2099 - skipped (1115 entries)

SEQ ID NO: 2100 - DNA, 1184 bp, synthetic construct
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 2101 - DNA, 227 bp, synthetic construct
  source 1..227: mol_type = other DNA
gcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2102 - skipped

SEQ ID NO: 2103 - DNA, 16 bp, synthetic construct
  source 1..16: mol_type = other DNA
ggtggagaagagcatg

SEQ ID NO: 2104 - DNA, 27 bp, synthetic construct
  source 1..27: mol_type = other DNA
gtcatcactgaggtggagaagagcatg

SEQ ID NO: 2105-2107 - skipped (3 entries)

SEQ ID NO: 2108 - DNA, 222 bp, synthetic construct
  source 1..222: mol_type = other DNA
gcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2109 - DNA, 222 bp, synthetic construct
  source 1..222: mol_type = other DNA
gcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgc

SEQ ID NO: 2110 - skipped

SEQ ID NO: 2111 - DNA, 229 bp, synthetic construct
  source 1..229: mol_type = other DNA
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2112 - DNA, 235 bp, synthetic construct
  source 1..235: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2113 - DNA, 241 bp, synthetic construct
  source 1..241: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2114 - DNA, 236 bp, synthetic construct
  source 1..236: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2115 - DNA, 246 bp, synthetic construct
  source 1..246: mol_type = other DNA
gcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2116 - DNA, 241 bp, synthetic construct
  source 1..241: mol_type = other DNA
gcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2117 - DNA, 257 bp, synthetic construct
  source 1..257: mol_type = other DNA
ggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2118 - DNA, 252 bp, synthetic construct
  source 1..252: mol_type = other DNA
ggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2119 - DNA, 268 bp, synthetic construct
  source 1..268: mol_type = other DNA
gtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaag

SEQ ID NO: 2120 - DNA, 263 bp, synthetic construct
  source 1..263: mol_type = other DNA
gtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2121-4672 - skipped (2552 entries)

SEQ ID NO: 4673 - DNA, 23 bp, unidentified
  source 1..23: mol_type = unassigned DNA
acaaacaccattgtcacactcca

SEQ ID NO: 4674 - DNA, 71 bp, unidentified
  source 1..71: mol_type = unassigned DNA
acaaacaccattgtcacactccacacaaacaccattgtcacactccacacaaacaccattgtcacactcca

SEQ ID NO: 4675 - DNA, 23 bp, unidentified
  source 1..23: mol_type = unassigned DNA
tccataaagtaggaaacactaca

SEQ ID NO: 4676 - DNA, 22 bp, unidentified
  source 1..22: mol_type = unassigned DNA
agtgaattctaccagtgccata

SEQ ID NO: 4677 - DNA, 24 bp, unidentified
  source 1..24: mol_type = unassigned DNA
agtgtgagttctaccattgccaaa

SEQ ID NO: 4678 - DNA, 23 bp, unidentified
  source 1..23: mol_type = unassigned DNA
agcaaaaatgtgctagtgccaaa

SEQ ID NO: 4679 - DNA, 22 bp, unidentified
  source 1..22: mol_type = unassigned DNA
atacatacttctttacattcca

(19) United States
(12) Patent Application Publication
(10) Pub. No.: US 2025/0215453 A1
(43) Pub. Date: Jul. 3, 2025

(54) AAV CAPSID VARIANTS AND USES THEREOF

(71) Applicant: VOYAGER THERAPEUTICS, INC., Lexington, MA (US)

(72) Inventors: Mathieu Emmanuel Nonnenmacher, Boston, MA (US); Jing Lin, Watertown, MA (US); Hongxing Wang, Newton, MA (US); Jinzhao Hou, Lexington, MA (US); Wei Wang, Arlington, MA (US); Jiangyu Li, Watertown, MA (US)

(21) Appl. No.: 18/876,521

(22) PCT Filed: Jun. 27, 2023
(86) PCT No.: PCT/US2023/069146

§ 371 Date: Dec. 18, 2024

Related U.S. Application Data
(60) Provisional application No. 63/356,337, filed on Jun. 28, 2022.

(51) Int. Cl. C12N 15/86; A61K 48/00; C07K 14/005

(52) U.S. Cl. CPC C12N 15/86; A61K 48/0041; C07K 14/005; C12N 2750/14122; C12N 2750/14143

(57) ABSTRACT

The disclosure relates to compositions and methods for the preparation, use, and/or formulation of adeno-associated virus capsid protein variants.

### RELATED APPLICATIONS

This application claims priority to U.S. Provisional Application No. 63/356,337 filed on Jun. 28, 2022; the entire contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Jun. 16, 2023, is named V2071-1124PCT_SL.xml and is 970,569 bytes in size.

### FIELD OF THE DISCLOSURE

The disclosure relates to compositions and methods for the preparation, use, and/or formulation of adeno-associated virus capsid proteins and variants thereof.

### BACKGROUND

Gene delivery to the central nervous system (CNS) remains a significant challenge in gene therapy. Engineered adeno-associated virus (AAV) capsids with improved brain tropism represent an attractive solution to the limitations of CNS delivery.

AAV-derived vectors are promising tools for clinical gene transfer because of their non-pathogenic nature, their low immunogenic profile, low rate of integration into the host genome and long-term transgene expression in non-dividing cells. However, the transduction efficiency of AAV natural variants in certain organs is too low for clinical applications, and capsid neutralization by pre-existing neutralizing antibodies may prevent treatment of a large proportion of patients. For these reasons, considerable efforts have been devoted to obtaining capsid variants with enhanced properties. Of many approaches tested so far, significant advances have resulted from directed evolution of AAV capsids using in vitro or in vivo selection of capsid variants created by capsid sequence randomization using either error-prone PCR, shuffling of various parent serotypes, or insertion of fully randomized short peptides at defined positions.

Attempts at providing AAV capsids with improved properties, e.g., improved tropism to a target cell or tissue upon systemic administration, have had limited success. As such, there is a need for improved methods of producing AAV capsids and resulting AAV capsids for delivery of a payload of interest to a target cell or tissue, e.g., a CNS cell or tissue, or a muscle cell or tissue.

### SUMMARY OF THE DISCLOSURE

The present disclosure pertains, at least in part, to compositions and methods for the production and use of an AAV particle comprising an AAV capsid polypeptide, e.g., an AAV capsid variant. In some embodiments, the AAV capsid variant has an enhanced tropism for a tissue or a cell, e.g., a CNS tissue or a CNS cell; or a muscle cell or tissue. Said tropism can be useful for delivery of a payload, e.g., a payload described herein, to a cell or tissue, for the treatment of a disorder, e.g., a neurological or a neurodegenerative disorder, a muscular disorder, a muscular dystrophy, a neuromuscular disorder, or a neuro-oncological disorder.

Accordingly, in one aspect, the present disclosure provides an AAV capsid variant, e.g., an AAV5 capsid variant, which comprises an amino acid other than T at position 578 (e.g., N), P at position 580 (e.g., A), A at position 581 (e.g., Q), T at position 582 (e.g., A), and/or G at position 583 (e.g., Y), numbered relative to SEQ ID NO: 138. In some embodiments, the AAV capsid variant comprises an N at position 578, numbered relative to SEQ ID NO: 138. In some embodiments, the AAV capsid variant comprises an A at position 580, numbered relative to SEQ ID NO: 138. In some embodiments, the AAV capsid variant comprises a Q at position 581, numbered relative to SEQ ID NO: 138. In some embodiments, the AAV capsid variant comprises an A at position 582, numbered relative to SEQ ID NO: 138. In some embodiments, the AAV capsid variant comprises a Y at position 583, numbered relative to SEQ ID NO: 138.

In yet another aspect, the present disclosure provides an AAV capsid variant comprising: (a) the amino acid sequence of SEQ ID NO: 943; (b) an amino acid sequence comprising at least 3, 4, or 5 consecutive amino acids from SEQ ID NO: 943; (c) an amino acid sequence comprising one, two, or three but no more than four different amino acids, relative to the amino acid sequence of SEQ ID NO: 943; or (d) an amino sequence comprising one, two, or three but no more than four modifications, e.g., substitutions (e.g., conservative substitutions), insertions, or deletions, relative to the amino acid sequence of SEQ ID NO: 943.

In yet another aspect, the present disclosure provides an AAV capsid variant comprising one, two, three, four, or all of an amino acid other than T at position 578, an amino acid other than P at position 580, an amino acid other than A at position 581, an amino acid other than T at position 582, and an amino acid other than G at position 583, numbered according to SEQ ID NO: 138.

In yet another aspect, the present disclosure provides an AAV capsid variant comprising an amino acid other than T at position 578, an amino acid other than P at position 580, an amino acid other than A at position 581, an amino acid other than T at position 582, and an amino acid other than G at position 583, numbered according to SEQ ID NO: 138.

In another aspect, the present disclosure provides an AAV capsid variant comprising one, two, three, four, five, or all of the amino acid N at position 578, A at position 579, A at position 580, Q at position 581, A at position 582, and/or Y at position 583, numbered according to SEQ ID NO: 138.

In yet another aspect, present disclosure provides an AAV capsid variant comprising one, two, three, four, or all of the amino acid N at position 578, A at position 580, Q at position 581, A at position 582, and/or Y at position 583, numbered according to SEQ ID NO: 138.

In yet another aspect, the present disclosure provides an AAV capsid variant comprising the amino acid N at position 578, A at position 579, A at position 580, Q at position 581, A at position 582, and Y at position 583, numbered according to SEQ ID NO: 138.

In yet another aspect, the present disclosure provides an AAV capsid variant comprising the amino acid N at position 578, A at position 580, Q at position 581, A at position 582, and Y at position 583, numbered according to SEQ ID NO: 138.

In yet another aspect, the present disclosure provides an AAV capsid variant comprising one, two, three, four, or all of the amino acid substitutions T578N, P580A, A581Q, T582A, and G583Y, numbered according to SEQ ID NO: 138.

In yet another aspect, the present disclosure provides an AAV capsid variant comprising the amino acid substitutions T578N, P580A, A581Q, T582A, and G583Y, numbered according to SEQ ID NO: 138.

In yet another aspect, the present disclosure provides an AAV capsid variant comprising (i) the amino acid sequence AQAY (SEQ ID NO: 1), optionally wherein the amino acid sequence replaces positions 580-583 (e.g., P580, A581, T582, G583), numbered according to SEQ ID NO: 138; and (ii) an amino acid other than T at position 578, numbered according to SEQ ID NO: 138.

In yet another aspect, the present disclosure provides an AAV capsid variant comprising (i) the amino acid sequence AQAY (SEQ ID NO: 1), wherein the amino acid sequence replaces positions 580-583 (e.g., P580, A581, T582, G583), numbered according to SEQ ID NO: 138; and (ii) an amino acid other than T at position 578, numbered according to SEQ ID NO: 138.

In yet another aspect, the present disclosure provides an AAV capsid variant comprising (i) the amino acid sequence AQAY (SEQ ID NO: 1), optionally wherein the amino acid sequence is present at positions 580-583, numbered according to SEQ ID NO: 982; and (ii) an amino acid other than T at position 578, numbered according to SEQ ID NO: 138.

In yet another aspect, the present disclosure provides an AAV capsid variant comprising (i) the amino acid sequence AQAY (SEQ ID NO: 1), wherein the amino acid sequence is present at positions 580-583, numbered according to SEQ ID NO: 982; and (ii) an amino acid other than T at position 578, numbered according to SEQ ID NO: 138.

In yet another aspect, the present disclosure provides an AAV capsid variant comprising (i) the amino acid sequence AQAY (SEQ ID NO: 1), optionally wherein the amino acid sequence replaces positions 580-583 (e.g., P580, A581, T582, G583), numbered according to SEQ ID NO: 138; and (ii) the amino acid N at position 578, numbered according to SEQ ID NO: 138.

In yet another aspect, the present disclosure provides an AAV capsid variant comprising (i) the amino acid sequence AQAY (SEQ ID NO: 1), wherein the amino acid sequence replaces positions 580-583 (e.g., P580, A581, T582, G583), numbered according to SEQ ID NO: 138; and (ii) the amino acid N at position 578, numbered according to SEQ ID NO: 138.

In yet another aspect, the present disclosure provides an AAV capsid variant comprising (i) the amino acid sequence AQAY (SEQ ID NO: 1), optionally wherein the amino acid sequence is present at positions 580-583, numbered according to SEQ ID NO: 982; and (ii) the amino acid N at position 578, numbered according to SEQ ID NO: 982.

In yet another aspect, the present disclosure provides an AAV capsid variant comprising (i) the amino acid sequence AQAY (SEQ ID NO: 1), wherein the amino acid sequence is present at positions 580-583, numbered according to SEQ ID NO: 982; and (ii) the amino acid N at position 578, numbered according to SEQ ID NO: 982.

In yet another aspect, the present disclosure provides an AAV capsid variant comprising the amino acid sequence of positions 193-724 of SEQ ID NO: 982, or an amino acid sequence at least 70%, 75%, 80%, 85%, 90%, 95%, 96%, 97%, 98%, or 99% identical thereto, wherein the AAV capsid variant comprises an N at position 578, A at position 580, Q at position 581, A at position 582, and Y at position 583, numbered according to SEQ ID NO: 982. In some embodiments, the AAV capsid variant comprises positions 193-724 of SEQ ID NO: 982. In some embodiments, the AAV capsid variant comprises positions 137-724 of SEQ ID NO: 982. In some embodiments the AAV capsid variant comprises the amino acid sequence of SEQ ID NO: 982.

In yet another aspect, the present disclosure provides an AAV capsid variant comprising the amino acid sequence of positions 137-724 of SEQ ID NO: 982, or an amino acid sequence at least 70%, 75%, 80%, 85%, 90%, 95%, 96%, 97%, 98%, or 99% identical thereto, wherein the AAV capsid variant comprises an N at position 578, A at position 580, Q at position 581, A at position 582, and Y at position 583, numbered according to SEQ ID NO: 982. In some embodiments, the AAV capsid variant comprises positions 137-724 of SEQ ID NO: 982. In some embodiments the AAV capsid variant comprises the amino acid sequence of SEQ ID NO: 982.

In yet another aspect, the present disclosure provides an AAV capsid variant comprising the amino acid sequence of SEQ ID NO: 982, or an amino acid sequence at least 70%, 75%, 80%, 85%, 90%, 95%, 96%, 97%, 98%, or 99% identical thereto, wherein the AAV capsid variant comprises an N at position 578, A at position 580, Q at position 581, A at position 582, and Y at position 583, numbered according to SEQ ID NO: 982. In some embodiments the AAV capsid variant comprises the amino acid sequence of SEQ ID NO: 982.

In another aspect, the present disclosure provides an AAV capsid variant comprising the amino acid sequence of SEQ ID NO: 982. In another aspect, the present disclosure provides an AAV capsid variant that consists of the amino acid sequence of SEQ ID NO: 982.

In yet another aspect, the present disclosure provides an AAV capsid variant comprising an amino acid sequence encoded by the nucleotide sequence of SEQ ID NO: 984. In another aspect, the present disclosure provides an AAV capsid variant comprising an amino acid sequence encoded by a nucleotide sequence having at least 95% identity to SEQ ID NO: 984.

In yet another aspect, the present disclosure provides a polynucleotide encoding an AAV capsid variant, wherein the encoded AAV capsid variant comprises: (a) the amino acid sequence of SEQ ID NO: 943; (b) an amino acid sequence comprising at least 3, 4, or 5 consecutive amino acids from SEQ ID NO: 943; (c) an amino acid sequence comprising one, two, or three but no more than four different amino acids, relative to the amino acid sequence of SEQ ID NO: 943; or (d) an amino sequence comprising one, two, or three but no more than four modifications, e.g., substitutions (e.g., conservative substitutions), insertions, or deletions, relative to the amino acid sequence of SEQ ID NO: 943.

In another aspect, the present disclosure provides a polynucleotide encoding an AAV capsid variant, wherein the encoded AAV capsid variant comprises: (i) one, two, three, four, or all of an amino acid other than T at position 578, an amino acid other than P at position 580, an amino acid other than A at position 581, an amino acid other than T at position 582, and/or an amino acid other than G at position 583, numbered according to SEQ ID NO: 138; (ii) an amino acid other than T at position 578, an amino acid other than P at position 580, an amino acid other than A at position 581, an amino acid other than T at position 582, and an amino acid other than G at position 583, numbered according to SEQ ID NO: 138; (iii) one, two, three, four, five, or all of the amino acid N at position 578, A at position 579, A at position 580, Q at position 581, A at position 582, and/or Y at position 583, numbered according to SEQ ID NO: 138; (iv) the amino acid N at position 578, A at position 579, A at position 580, Q at position 581, A at position 582, and Y at position 583, numbered according to SEQ ID NO: 138; (v) one, two, three, four, or all of the amino acid N at position 578, A at position 580, Q at position 581, A at position 582, and/or Y at position 583, numbered according to SEQ ID NO: 138; (vi) the amino acid N at position 578, A at position 580, Q at position 581, A at position 582, and Y at position 583, numbered according to SEQ ID NO: 138; (vii) one, two, three, four, or all of the amino acid substitutions T578N, P580A, A581Q, T582A, and/or G583Y, numbered according to SEQ ID NO: 138; and/or (viii) the amino acid substitutions T578N, P580A, A581Q, T582A, and G583Y, numbered according to SEQ ID NO: 138.

In yet another aspect, the present disclosure provides a polynucleotide encoding an AAV capsid variant comprising: (i) the amino acid sequence AQAY (SEQ ID NO: 1), optionally wherein the amino acid sequence is present at positions 580-583, numbered according to SEQ ID NO: 982; and an amino acid other than T at position 578, numbered according to SEQ ID NO: 138; and/or (ii) the amino acid sequence AQAY (SEQ ID NO: 1), optionally wherein the amino acid sequence is present at positions 580-583, numbered according to SEQ ID NO: 982; and the amino acid N at position 578, numbered according to SEQ ID NO: 138.

In yet another aspect, the present disclosure provides a polynucleotide encoding an AAV capsid variant comprising: (i) the amino acid sequence AQAY (SEQ ID NO: 1), optionally wherein the amino acid sequence corresponds to positions 580-583 of SEQ ID NO: 982; and an amino acid other than T at position 578, numbered according to SEQ ID NO: 138; and/or (ii) the amino acid sequence AQAY (SEQ ID NO: 1), optionally wherein the amino acid sequence corresponds to positions 580-583 of SEQ ID NO: 982; and the amino acid N at position 578, numbered according to SEQ ID NO: 138.

In yet another aspect, the present disclosure provides a peptide comprising: (a) the amino acid sequence of SEQ ID NO: 943; (b) an amino acid sequence comprising at least 3, 4, or 5 consecutive amino acids from SEQ ID NO: 943; or (c) an amino acid sequence comprising one, two, or three but no more than four different amino acids, relative to the amino acid sequence of SEQ ID NO: 943; or (d) an amino sequence comprising one, two, or three but no more than four modifications, e.g., substitutions (e.g., conservative substitutions), insertions, or deletions, relative to the amino acid sequence of SEQ ID NO: 943.

In yet another aspect, the present disclosure provides a peptide comprising the amino acid sequence of NAAQAY (SEQ ID NO: 943).

In yet another aspect, the present disclosure provides a peptide comprising the amino acid sequence of ATNNQSSTNAAQAYT (SEQ ID NO: 744).

In yet another aspect, the present disclosure provides a peptide encoded by the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto. In yet another aspect, the present disclosure provides a peptide encoded by (i) a nucleotide sequence comprising one, two, three, four, five, six, or seven, but no more than ten different nucleotides, relative to the nucleotide sequence of SEQ ID NO: 944; or (ii) a nucleotide sequence comprising one, two, three, four, five, six, or seven modifications, e.g., substitutions, insertions or deletions, but no more than ten modifications, e.g., substitutions, insertions or deletions, relative to the nucleotide sequence of SEQ ID NO: 944.

In yet another aspect, the present disclosure provides a peptide, wherein the nucleotide sequence encoding the peptide comprises (i) the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto; (ii) a nucleotide sequence comprising one, two, three, four, five, six, or seven, but no more than ten different nucleotides relative to the nucleotide sequence of SEQ ID NO: 944; or (iii) a nucleotide sequence comprising one, two, three, four, five, six, or seven modifications, e.g., substitutions, insertions or deletions, but no more than ten modifications, e.g., substitutions, insertions or deletions, relative to the nucleotide sequence of SEQ ID NO: 944.

In yet another aspect, the present disclosure provides an AAV particle comprising an AAV capsid variant described herein. In some embodiments, the AAV particle comprises a nucleic acid sequence encoding a payload. In some embodiments, the AAV particle further comprises a viral genome comprising a promoter operably linked to the nucleic acid sequence encoding the payload.

In yet another aspect, the present disclosure provides a method of making an AAV particle comprising an AAV capsid polypeptide, e.g., an AAV capsid variant, described herein. In some embodiments, the method comprises providing a host cell comprising a viral genome and incubating the host cell under conditions suitable to enclose the viral genome in the AAV capsid variant, e.g., an AAV capsid variant described herein, thereby making the AAV particle.

In yet another aspect, the present disclosure provides a method of delivering a payload to a cell or tissue (e.g., a CNS cell or a CNS tissue). The method comprising administering an effective amount of an AAV particle comprising an AAV capsid variant described herein.

In yet another aspect, the present disclosure provides a method of treating a subject having or diagnosed with having a genetic disorder e.g., a monogenic disorder or a polygenic disorder. The method comprising administering an effective amount of an AAV particle comprising an AAV capsid variant described herein.

In yet another aspect, the present disclosure provides a method of treating a subject having or diagnosed with having a neurological, e.g., a neurodegenerative, disorder. The method comprising administering an effective amount of an AAV particle comprising an AAV capsid variant described herein.

In yet another aspect, the present disclosure provides a method of treating a subject having or diagnosed with having a muscular disorder, a muscular dystrophy, or a neuromuscular disorder. The method comprising administering an effective amount of an AAV particle comprising an AAV capsid variant described herein.

In yet another aspect, the present disclosure provides a method of treating a subject having or diagnosed with having a cardiac disorder, e.g., a cardiac disorder as described herein (e.g., a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction). The method comprising administering an effective amount of an AAV particle comprising an AAV capsid variant described herein.

In yet another aspect, the present disclosure provides a method of treating a subject having or diagnosed with having a neuro-oncological disorder. The method comprising administering an effective amount of an AAV particle comprising an AAV capsid variant described herein.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. An AAV capsid variant comprising:
   (a) the amino acid sequence of SEQ ID NO: 943;
   (b) an amino acid sequence comprising at least 3, 4, or 5 consecutive amino acids from SEQ ID NO: 943;
   (c) an amino acid sequence comprising one, two, or three but no more than four different amino acids, relative to the amino acid sequence of SEQ ID NO: 943; or
   (d) an amino sequence comprising one, two, or three but no more than four modifications, e.g., substitutions (e.g., conservative substitutions), insertions, or deletions, relative to the amino acid sequence of SEQ ID NO: 943.
2. The AAV capsid variant of embodiment 1, which comprises at least 3, 4, or 5 consecutive amino acids from SEQ ID NO: 943.
3. The AAV capsid variant of embodiment 1 or 2, wherein the 3 consecutive amino acids comprise NAA.
4. The AAV capsid variant of any one of embodiments 1-3, wherein the 4 consecutive amino acids comprise NAAQ (SEQ ID NO: 2).
5. The AAV capsid variant of any one of embodiments 1-4, wherein the 5 consecutive amino acids comprise NAAQA (SEQ ID NO: 3).
6. The AAV capsid variant of any one of embodiments 1-5, wherein amino acid sequence comprises NAAQAY (SEQ ID NO: 943).
7. The AAV capsid variant of any one of embodiments 1-6, which comprises an amino acid sequence comprising one, two, or three but no more than four different amino acids, relative to the amino acid sequence of NAAQAY (SEQ ID NO: 943).
8. The AAV capsid variant of any one of embodiments 1-7, which comprises an amino acid sequence comprising one or two (e.g., no more than two) different amino acids, relative to the amino acid sequence of NAAQAY (SEQ ID NO: 943).
9. The AAV capsid variant of any one of embodiments 1-8, which comprises an amino acid sequence comprising one, two, or three but no more than four modifications, e.g., substitutions (e.g., conservative substitutions), insertions, or deletions, relative to the amino acid sequence of NAAQAY (SEQ ID NO: 943).
10. The AAV capsid variant of any one of embodiments 1-9, which comprises an amino acid sequence comprising one or two (e.g., no more than two) modifications, e.g., substitutions (e.g., conservative substitutions), insertions, or deletions, relative to the amino acid sequence of NAAQAY (SEQ ID NO: 943).
11. The AAV capsid variant of any one of embodiments 1-10, which comprises an amino acid sequence encoded by:
   (i) a nucleotide sequence comprising at least one, two, three, four, five, six, or seven modifications, e.g., substitutions, but no more than ten modifications, e.g., substitutions, relative to the nucleotide sequence of SEQ ID NO: 944; or
   (ii) a nucleotide sequence comprising at least one, two, three, four, five, six, or seven, but no more than ten different nucleotides relative to the nucleotide sequence of SEQ ID NO: 944.
12. The AAV capsid variant of any one of embodiments 1-11, wherein the nucleotide sequence encoding the amino acid sequence comprises:
   (i) a nucleotide sequence comprising at least one, two, three, four, five, six, or seven modifications, e.g., substitutions, but no more than ten modifications, e.g., substitutions, relative to the nucleotide sequence of SEQ ID NO: 944; or
   (ii) a nucleotide sequence comprising at least one, two, three, four, five, six, or seven, but no more than ten different nucleotides relative to the nucleotide sequence of SEQ ID NO: 944.
13. The AAV capsid variant of any one of embodiments 1-12, wherein the amino acid sequence is present in loop VIII, optionally wherein loop VIII comprises positions 571-592, numbered according to SEQ ID NO: 138.
14. The AAV capsid variant of any one of embodiments 1-13, wherein the amino acid sequence replaces one, two, three, four, five, or all of positions 578, 579, 580, 581, 582, and/or 583 (e.g., positions T578, A579, P580, A581, T582, and/or G583), numbered according to the amino acid sequence of SEQ ID NO: 138.
15. The AAV capsid variant of any one of embodiments 1-14, wherein the amino acid sequence replaces positions 578, 579, 580, 581, 582, and 583 (e.g., positions T578, A579, P580, A581, T582, and G583), numbered according to the amino acid sequence of SEQ ID NO: 138.
16. The AAV capsid variant of any one of embodiments 1-15, wherein the amino acid sequence corresponds to positions 578, 579, 580, 581, 582, and 583 (e.g., positions N578, A579, A580, Q581, A582, and Y583) of SEQ ID NO: 982.
17. The AAV capsid variant of any one of embodiments 1-16, which comprises the amino acid sequence of NAAQAY (SEQ ID NO: 943), optionally wherein the amino acid sequence replaces positions 578, 579, 580, 581, 582, 583 (e.g., positions T578, A579, P580, A581, T582, and G583), relative to a reference sequence numbered according to the amino acid sequence of SEQ ID NO: 138.
18. The AAV capsid variant of any one of embodiments 1-17, which comprises the amino acid sequence of NAAQAY (SEQ ID NO: 943), optionally wherein the amino acid sequence corresponds to positions 578, 579, 580, 581, 582, 583 (e.g., positions N578, A579, A580, Q581, A582, and Y583) of SEQ ID NO: 982.
19. The AAV capsid variant of any one of embodiments 1-18, which comprises the amino acid sequence of NAAQAY (SEQ ID NO: 943), optionally wherein the amino acid sequence is present at positions 578-583, numbered according to SEQ ID NO: 982.
20. The AAV capsid variant of any one of embodiments 1-16, which comprises the amino acid sequence AQAY (SEQ ID NO: 1), optionally wherein the amino acid sequence replaces positions 580-583 (e.g., P580, A581, T582, and G583), numbered relative to SEQ ID NO: 138.
21. The AAV capsid variant of any one of embodiments 1-16, which comprises the amino acid sequence AQAY (SEQ ID NO: 1), optionally wherein the amino acid sequence is present at positions 580-583, numbered relative to SEQ ID NO: 982.
22. The AAV capsid variant of embodiment 20 or 21, which further comprises an amino acid other than T at position 578, numbered according to SEQ ID NO: 138.
23. The AAV capsid variant of any one of embodiments 20-22, which further comprises the amino acid N at position 578, numbered according to SEQ ID NO: 138.
24. The AAV capsid variant of any one of embodiments 1-23, which comprises:
   (i) the amino acid sequence AQAY (SEQ ID NO: 1), wherein the amino acid sequence replaces positions 580-583 (e.g., P580, A581, T582, and G583), numbered relative to SEQ ID NO: 138; and/or
   (ii) an amino acid other than T at position 578, numbered according to SEQ ID NO: 138.
25. The AAV capsid variant of any one of embodiments 1-24, which comprises:
   (i) the amino acid sequence AQAY (SEQ ID NO: 1), wherein the amino acid sequence is present at positions 580-583, numbered according to SEQ ID NO: 982; and/or
   (ii) an amino acid other than T at position 578, numbered according to SEQ ID NO: 138.
26. The AAV capsid variant of any one of embodiments 1-25, which comprises:
   (i) the amino acid sequence AQAY (SEQ ID NO: 1), wherein the amino acid sequence replaces positions 580-583 (e.g., P580, A581, T582, and G583), numbered relative to SEQ ID NO: 138; and/or
   (ii) the amino acid N at position 578, numbered according to SEQ ID NO: 138.
27. The AAV capsid variant of any one of embodiments 1-26, which comprises:
   (i) the amino acid sequence AQAY (SEQ ID NO: 1), wherein the amino acid sequence is present at positions 580-583, numbered according to SEQ ID NO: 982; and/or
   (ii) the amino acid N at position 578, numbered according to SEQ ID NO: 982.
28. The AAV capsid variant of any one of embodiments 1-27, which comprises:
   (i) the amino acid sequence AQAY (SEQ ID NO: 1), wherein the amino acid sequence corresponds to positions 580-583 of SEQ ID NO: 982; and/or
   (ii) the amino acid N at position 578, numbered according to SEQ ID NO: 138.
29. The AAV capsid variant of any one of the preceding embodiments, which comprises one, two, three, four, or all of an amino acid other than T at position 578, an amino acid other than P at position 580, an amino acid other than A at position 581, an amino acid other than T at position 582, and/or an amino acid other than G at position 583, numbered according to SEQ ID NO: 138.
30. An AAV capsid variant which comprises one, two, three, four, five, or all of an amino acid other than T at position 578, an amino acid other than P at position 580, an amino acid other than A at position 581, an amino acid other than T at position 582, and/or an amino acid other than G at position 583, numbered according to SEQ ID NO: 138.
31. The AAV capsid variant of any one of the preceding embodiments, which comprises an amino acid other than T at position 578, an amino acid other than P at position 580, an amino acid other than A at position 581, an amino acid other than T at position 582, and an amino acid other than G at position 583, numbered according to SEQ ID NO: 138.
32. An AAV capsid variant comprising an amino acid other than T at position 578, an amino acid other than P at position 580, an amino acid other than A at position 581, an amino acid other than T at position 582, and an amino acid other than G at position 583, numbered according to SEQ ID NO: 138.
33. The AAV capsid variant of any one of the preceding embodiments, which comprises one, two, three, four, five, or all of the amino acid N at position 578, A at position 579, A at position 580, Q at position 581, A at position 582, and/or Y at position 583, numbered according to SEQ ID NO: 138.
34. The AAV capsid variant of any one of the preceding embodiments, which comprises one, two, three, four, or all of the amino acid N at position 578, A at position 580, Q at position 581, A at position 582, and/or Y at position 583, numbered according to SEQ ID NO: 138.
35. An AAV capsid variant comprising one, two, three, four, five, or all of the amino acid N at position 578, A at position 579, A at position 580, Q at position 581, A at position 582, and/or Y at position 583, numbered according to SEQ ID NO: 138.
36. An AAV capsid variant comprising one, two, three, four, or all of the amino acid N at position 578, A at position 580, Q at position 581, A at position 582, and/or Y at position 583, numbered according to SEQ ID NO: 138.
37. The AAV capsid variant of any one of the preceding embodiments, which comprises the amino acid N at position 578, A at position 579, A at position 580, Q at position 581, A at position 582, and Y at position 583, numbered according to SEQ ID NO: 138.
38. The AAV capsid variant of any one of the preceding embodiments, which comprises the amino acid N at position 578, A at position 580, Q at position 581, A at position 582, and Y at position 583, numbered according to SEQ ID NO: 138.
39. An AAV capsid variant comprising the amino acid N at position 578, A at position 579, A at position 580, Q at position 581, A at position 582, and Y at position 583, numbered according to SEQ ID NO: 138.
40. An AAV capsid variant comprising the amino acid N at position 578, A at position 580, Q at position 581, A at position 582, and Y at position 583, numbered according to SEQ ID NO: 138.
41. The AAV capsid variant of any one of embodiments 1-32, which comprises one, two, three, four, or all of the amino acid substitutions T578N, P580A, A581Q, T582A, and/or G583Y, numbered according to SEQ ID NO: 138.
42. An AAV capsid variant comprising one, two, three, four, or all of the amino acid substitutions T578N, P580A, A581Q, T582A, and/or G583Y, numbered according to SEQ ID NO: 138.
43. The AAV capsid variant of any one of embodiments 1-38, which comprises the amino acid substitutions T578N, P580A, A581Q, T582A, and G583Y, numbered according to SEQ ID NO: 138.
44. An AAV capsid variant comprising the amino acid substitutions T578N, P580A, A581Q, T582A, and G583Y, numbered according to SEQ ID NO: 138.
45. The AAV capsid variant of any one of the preceding embodiments, which corresponds to positions 578-583 (e.g., N578, A579, A580, Q581, A582, Y583) of SEQ ID NO: 982.
46. An AAV capsid variant which comprises:
   (i) the amino acid sequence AQAY (SEQ ID NO: 1), optionally wherein the amino acid sequence replaces positions 580-583 (e.g., P580, A581, T582, G583), numbered according to SEQ ID NO: 138; and
   (ii) an amino acid other than T at position 578, numbered according to SEQ ID NO: 138.
47. An AAV capsid variant which comprises:
   (i) the amino acid sequence AQAY (SEQ ID NO: 1), wherein the amino acid sequence replaces positions 580-583 (e.g., P580, A581, T582, G583), numbered according to SEQ ID NO: 138; and
   (ii) an amino acid other than T at position 578, numbered according to SEQ ID NO: 138.
48. An AAV capsid variant which comprises:
   (i) the amino acid sequence AQAY (SEQ ID NO: 1), optionally wherein the amino acid sequence is present at positions 580-583, numbered according to SEQ ID NO: 982; and
   (ii) an amino acid other than T at position 578, numbered according to SEQ ID NO: 138.
49. An AAV capsid variant which comprises:
   (i) the amino acid sequence AQAY (SEQ ID NO: 1), wherein the amino acid sequence is present at positions 580-583, numbered according to SEQ ID NO: 982; and
   (ii) an amino acid other than T at position 578, numbered according to SEQ ID NO: 138.
50. An AAV capsid variant which comprises:
   (i) the amino acid sequence AQAY (SEQ ID NO: 1), optionally wherein the amino acid sequence replaces positions 580-583 (e.g., P580, A581, T582, G583), numbered according to SEQ ID NO: 138; and
   (ii) the amino acid other N at position 578, numbered according to SEQ ID NO: 138.
51. An AAV capsid variant which comprises:
   (i) the amino acid sequence AQAY (SEQ ID NO: 1), optionally wherein the amino acid sequence replaces positions 580-583 (e.g., P580, A581, T582, G583), numbered according to SEQ ID NO: 138; and
   (ii) the amino acid other N at position 578, numbered according to SEQ ID NO: 138.
52. An AAV capsid variant which comprises:
   (i) the amino acid sequence AQAY (SEQ ID NO: 1), optionally wherein the amino acid sequence is present at positions 580-583, numbered according to SEQ ID NO: 982; and
   (ii) the amino acid other N at position 578, numbered according to SEQ ID NO: 982.
53. An AAV capsid variant which comprises:
   (i) the amino acid sequence AQAY (SEQ ID NO: 1), wherein the amino acid sequence is present at positions 580-583, numbered according to SEQ ID NO: 982; and
   (ii) the amino acid other N at position 578, numbered according to SEQ ID NO: 982.
54. The AAV capsid variant of any one of the preceding embodiments, which comprises the amino acid A at position 579, numbered according to SEQ ID NO: 138 or 982.
55. The AAV capsid variant of any one of the preceding embodiments, which corresponds to position 579 of SEQ ID NO: 138 or 982.
56. The AAV capsid variant, of any one of the preceding embodiments, which further comprises a modification, e.g., an insertion, substitution (e.g., conservative substitution), and/or deletion, in loop I, II, IV, and/or VI.
57. The AAV capsid variant of any one of the preceding embodiments, which comprises an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 138.
58. The AAV capsid variant, of any one of the preceding embodiments, which comprises an amino acid sequence comprising at least one, two or three, but no more than 30, 20 or 10 different amino acids relative to the amino acid sequence of SEQ ID NO: 138.
59. The AAV capsid variant, of any one of the preceding embodiments, which comprises the amino acid sequence of SEQ ID NO: 138, or an amino acid sequence with at least 80% (e.g., at least about 85, 90, 95, 96, 97, 98, or 99%) sequence identity thereto.
60. The AAV capsid variant, of any one of the preceding embodiments, which comprises the amino acid sequence of SEQ ID NO: 138.
61. The AAV capsid variant, of any one of the preceding embodiments, which comprises an amino acid sequence encoded by the nucleotide sequence of SEQ ID NO: 137, or a sequence with at least 80% (e.g., at least about 85, 90, 95, 96, 97, 98, or 99%) sequence identity thereto.
62. The AAV capsid variant, of any one of the preceding embodiments, wherein the nucleotide sequence encoding the capsid variant comprises the nucleotide sequence of SEQ ID NO: 137, or a sequence with at least 80% (e.g., at least about 85, 90, 95, 96, 97, 98, or 99%) sequence identity thereto.
63. The AAV capsid variant, of any one of the preceding embodiments, which comprises a VP1 protein, a VP2 protein, a VP3 protein, or a combination thereof.
64. The AAV capsid variant, of any one of embodiments 1-63, which comprises the amino acid sequence corresponding to positions 137-724, e.g., a VP2, of SEQ ID NO: 982, or a sequence with at least 80% (e.g., at least about 85, 90, 95, 96, 97, 98, or 99%) sequence identity thereto.
65. The AAV capsid variant, of any one of embodiments 1-64, which comprises the amino acid sequence corresponding to positions 193-724, e.g., a VP3, of SEQ ID NO: 982, or a sequence with at least 80% (e.g., at least about 85, 90, 95, 96, 97, 98, or 99%) sequence identity thereto.
66. The AAV capsid variant, of any one of embodiments 1-65, which comprises the amino acid sequence corresponding to positions 137-724, e.g., a VP2, of SEQ ID NO: 138, or a sequence with at least 80% (e.g., at least about 85, 90, 95, 96, 97, 98, or 99%) sequence identity thereto.
67. The AAV capsid variant, of any one of embodiments 1-66, which comprises the amino acid sequence corresponding to positions 193-724, e.g., a VP3, of SEQ ID NO: 138, or a sequence with at least 80% (e.g., at least about 85, 90, 95, 96, 97, 98, or 99%) sequence identity thereto.
68. The AAV capsid variant, of any one of embodiments 1-67, comprising an amino acid sequence comprising at least 3, 4, 5, or 6 consecutive amino acids from the amino acid sequence of NAAQAY (SEQ ID NO: 943), wherein:
   (i) the 3 consecutive amino acids comprise NAA;
   (ii) the 4 consecutive amino acids comprise NAAQ (SEQ ID NO: 2);
   (iii) the 5 consecutive amino acids comprise NAAQA (SEQ ID NO: 3);
   (iv) the 6 consecutive amino acids comprise NAAQAY (SEQ ID NO: 943);
   wherein the AAV capsid variant comprises: (a) a VP1 protein comprising the amino acid sequence of SEQ ID NO: 138 or SEQ ID NO: 982; (b) a VP2 protein comprising the amino acid sequence of positions 137-724 of SEQ ID NO: 138 or positions 137-724 of SEQ ID NO: 982; (c) a VP3 protein comprising the amino acid sequence of positions 193-724 of SEQ ID NO: 138 or positions 193-724 of SEQ ID NO: 982; or (d) an amino acid sequence with at least 90% (e.g., at least about 95, 96, 97, 98, or 99%) sequence identity to any of the amino acid sequences in (a)-(c).
69. The AAV capsid variant, of any one of embodiments 1-68, comprising one, two, or three but no more than four different amino acids, relative to the amino acid sequence of NAAQAY (SEQ ID NO: 943), wherein the AAV capsid variant comprises:
   (a) a VP1 protein comprising the amino acid sequence of SEQ ID NO: 138 or SEQ ID NO: 982;
   (b) a VP2 protein comprising the amino acid sequence of positions 137-724 of SEQ ID NO: 138 or positions 137-724 of SEQ ID NO: 982;
   (c) a VP3 protein comprising the amino acid sequence of positions 193-724 of SEQ ID NO: 138 or positions 193-724 of SEQ ID NO: 982; or
   (d) an amino acid sequence with at least 90% (e.g., at least about 95, 96, 97, 98, or 99%) sequence identity to any of the amino acid sequences in (a)-(c).
70. The AAV capsid variant, of any one of embodiments 1-69, comprising one or two, but no more than three substitutions relative to the amino acid sequence of NAAQAY (SEQ ID NO: 943), wherein the AAV capsid variant comprises an amino acid sequence at least 90% (e.g., at least about 95, 96, 97, 98, or 99%) identical to the amino acid sequence of SEQ ID NO: 982.
71. The AAV capsid variant, of any one of embodiments 1-70, which comprises the amino acid sequence of SEQ ID NO: 982, or an amino acid sequence with at least 80% (e.g., at least about 85, 90, 95, 96, 97, 98, or 99%) sequence identity thereto.
72. The AAV capsid variant, of any one of embodiments 1-71, which comprises the amino acid sequence of SEQ ID NO: 982 or an amino acid sequence with at least 95% identity thereto.
73. The AAV capsid variant, of any one of embodiments 1-72, which comprises the amino acid sequence of SEQ ID NO: 982 or an amino acid sequence with at least 98% identity thereto.
74. The AAV capsid variant, of any one of embodiments 1-73, which comprises the amino acid sequence of SEQ ID NO: 982 or an amino acid sequence with at least 99% identity thereto.
75. The AAV capsid variant of any one of embodiments 1-74, which comprises an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 982.
76. The AAV capsid variant, of any one of embodiments 1-75, which comprises an amino acid sequence comprising at least one, two or three, but not more than 30, 20 or 10 different amino acids, relative to the amino acid sequence of SEQ ID NO: 982.
77. The AAV capsid variant, of any one of embodiments 1-76, which comprises the amino acid sequence of SEQ ID NO: 982.
78. The AAV capsid variant, of any one of the preceding embodiments 1-77, wherein the nucleotide sequence encoding the capsid variant comprises the nucleotide sequence of SEQ ID NO: 984, or a nucleotide sequence with at least 80% (e.g., at least about 85, 90, 95, 96, 97, 98, or 99%) sequence identity thereto.
79. The AAV capsid variant, of any one of the preceding embodiments, wherein the nucleotide sequence encoding the capsid variant is codon optimized.
80. An AAV capsid variant comprising the amino acid sequence of any one of embodiments 1-28, and further comprising an amino acid sequence at least 80% (e.g., at least about 85, 90, 95, 96, 97, 98, or 99%) identical to SEQ ID NO: 982.
81. An AAV capsid variant comprising an amino acid sequence at least 70%, 75%, 80%, 85%, 90%, 95%, 96%, 97%, 98%, or 99% identical to positions 193-724 of SEQ ID NO: 982, wherein the AAV capsid variant comprises an N at position 578, A at position 580, Q at position 581, A at position 582, and Y at position 583, numbered according to SEQ ID NO: 982.
82. The AAV capsid variant of embodiment 81, which comprises the amino acid sequence of positions 193-724 of SEQ ID NO: 982.
83. An AAV capsid variant comprising an amino acid sequence at least 70%, 75%, 80%, 85%, 90%, 95%, 96%, 97%, 98%, or 99% identical to positions 137-724 of SEQ ID NO: 982, wherein the AAV capsid variant comprises an N at position 578, A at position 580, Q at position 581, A at position 582, and Y at position 583, numbered according to SEQ ID NO: 982.
84. The AAV capsid variant of any one of embodiments 81-83, which comprises the amino acid sequence of positions 137-724 of SEQ ID NO: 982.
85. An AAV capsid variant comprising an amino acid sequence at least 70%, 75%, 80%, 85%, 90%, 95%, 96%, 97%, 98%, or 99% identical to SEQ ID NO: 982, wherein the AAV capsid variant comprises an N at position 578, A at position 580, Q at position 581, A at position 582, and Y at position 583, numbered according to SEQ ID NO: 982.
86. The AAV capsid variant of any one of embodiments 81-85, which comprises the amino acid sequence of SEQ ID NO: 982.
87. An AAV capsid variant comprising the amino acid sequence of SEQ ID NO: 982.
88. An AAV capsid variant comprising an amino acid sequence encoded by the nucleotide sequence of SEQ ID NO: 984, or a nucleotide sequence at least 95% identical thereto.
89. The AAV capsid variant of any one of embodiments 81-88, wherein the nucleotide sequence encoding the AAV capsid variant comprises the nucleotide sequence of SEQ ID NO: 984, or a nucleotide sequence at least 95% identical thereto.
90. The AAV capsid variant of any one embodiments 1-89, which has an increased tropism for a muscle cell or tissue, relative to the tropism of a reference sequence comprising the amino acid sequence of SEQ ID NO: 138 or SEQ ID NO: 139.
91. The AAV capsid variant of any one of embodiments 1-90, which transduces a muscle region, optionally wherein the level of transduction is at least 2, 5, 10, 15, 20, or 25-fold greater as compared to a reference sequence of SEQ ID NO: 138 or 139, e.g., when measured by an assay, e.g., an immunohistochemistry assay or a qPCR assay, e.g., as described in Example 3.
92. The AAV capsid variant of any one of embodiments 1-91, which delivers an increased level of a payload to a muscle cell or region, optionally wherein the level of the payload is increased by at least 5, 10, 12, 15, 20, 21, or 25-fold, as compared to a reference sequence of SEQ ID NO: 138 or SEQ ID NO: 139, e.g., when measured by an assay, e.g., a qRT-PCR or a qPCR assay (e.g., as described in Example 3.
93. The AAV capsid variant of any one of embodiments 1-92, which delivers an increased level of viral genomes to a muscle cell or region, optionally wherein the level of viral genomes is increased by at least 2, 2.5, 5, 5.5, 6, 6.5, 7, 7.5, 8, 8.5, 9, 9.5, 10, 10.5, 11, 11.5, 12, 12.5, 13, 13.5, 13.8, or 14-fold, as compared to a reference sequence of SEQ ID NO: 138 or SEQ ID NO: 139, e.g., when measured by an assay, e.g., a qRT-PCR or a qPCR assay (e.g., as described in Example 3).
94. The AAV capsid variant of any one of embodiments 1-93, which has increased tropism for a heart cell or tissue, relative to the tropism of a reference sequence of SEQ ID NO: 138 or SEQ ID NO: 139.
95. The AV capsid variant of any one of embodiments 1-94, which delivers an increased level of a payload to a heart cell or region, optionally wherein the level of the payload is increased by at least 2, 2.5, 3, 3.5, 4, 4.5, or 5-fold, as compared to a reference sequence of SEQ ID NO: 138, e.g., when measured by an assay, e.g., a qRT-PCR or a qPCR assay (e.g., as described in Example 3).
96. The AAV capsid variant of any one of embodiments 1-95, which delivers an increased level of viral genomes to a heart cell or region, optionally wherein the level of viral genomes is increased by at least 5, 6, 7, 8, 9 or 10-fold, as compared to a reference sequence of SEQ ID NO: 138 or SEQ ID NO: 139, e.g., when measured by an assay, e.g., a qRT-PCR or a qPCR assay (e.g., as described in Example 3).
97. The AAV capsid variant of any one embodiments 1-96, which has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138 or SEQ ID SEQ ID NO: 139.
98. The AAV capsid variant of any one of embodiments 1-97, which transduces a brain cell or region, e.g., (e.g., the caudate, motor cortex, putamen, thalamus, and/or cerebellum (e.g., the molecular and granule layer of the cerebellum)), optionally wherein the level of transduction is at least 3, 4, 5, 6, 7, 8, 9, 10, 11, 12, 13, 14, 15, 20, 24, 25, 29, or 30-fold greater as compared to a reference sequence of SEQ ID NO: 138 or SEQ ID NO: 139, e.g., when measured by an assay, e.g., an immunohistochemistry assay or a qPCR assay, e.g., as described in Example 3.
99. The AAV capsid variant of any one of embodiments 1-98, which delivers an increased level of a payload to a brain cell or region, optionally wherein the level of the payload is increased by at least 3, 4, 5, 6, 7, 8, 9, 10, 11, 12, 13, or 14-fold, as compared to a reference sequence of SEQ ID NO: 138 or SEQ ID NO: 139, e.g., when measured by an assay, e.g., a qRT-PCR or a qPCR assay (e.g., as described in Example 3).
100. The AAV capsid variant of any one of embodiments 1-99, which delivers an increased level of viral genomes to a brain cell or region, optionally wherein the level of viral genomes is increased by at least 3, 4, 5, 6, 7, 8, 9, 10, 11, 12, 13, 14, 15, 20, 24, 25, 29, or 30-fold, as compared to a reference sequence of SEQ ID NO: 138 or SEQ ID NO: 139, e.g., when measured by an assay, e.g., a qRT-PCR or a qPCR assay (e.g., as described in Example 3).
101. The AAV capsid variant of any one of embodiments 98-100, wherein the brain region is a caudate, motor cortex, putamen, thalamus, and/or cerebellum (e.g., the molecular and granule layer of the cerebellum).
102. The AAV capsid variant of any one of embodiments 1-101, which is enriched at least about 4, 4.6, 10, 20, 25, 28, 30, 40, 50, 60, 70, 80, 81, 80, 100, 101, or 110-fold, in the brain compared to a reference sequence of SEQ ID NO: 138, e.g., when measured by an assay as described in Example 1 or 2.
103. The AAV capsid variant of any one of embodiments 1-102, which is enriched in the brain of at least two to three species, e.g., a non-human primate and rodent (e.g., mouse), e.g., as compared to a reference sequence of SEQ ID NO: 138.
104. The AAV capsid variant, of any one of embodiments 1-103, which is enriched at least about 4, 4.6, 10, 20, 25, 28, 30, 40, 50, 60, 70, 80, 81, 80, 100, 101, or 110-fold, in the brain of at least two to three species, e.g., a non-human primate and rodent (e.g., mouse), compared to a reference sequence of SEQ ID NO: 138, e.g., when measured by an assay as described in Examples 1 and 2.
105. The AAV capsid variant of embodiment 103 or 104, wherein the at least two to three species are Macaca fascicularis, Chlorocebus sabaeus, Callithrix jacchus, and/or mouse (e.g., BALB/c and/or C57BL6 mice).
106. The AAV capsid variant of any one of embodiments 1-105, which shows preferential transduction in a muscle cell or region relative to the transduction in the liver.
107. The AAV capsid variant of any one of embodiments 1-106, which shows preferential transduction in a heart cell or region relative to the transduction in the liver.
108. The AAV capsid variant of any one embodiments 1-107, which has an decreased tropism for a liver cell or tissue, relative to the tropism of a reference sequence comprising the amino acid sequence of SEQ ID NO: 138 or SEQ ID SEQ ID NO: 139.
109. The AAV capsid variant of any one of embodiments 1-108, which shows preferential transduction in a brain cell or region relative to the transduction in the liver.
110. A polynucleotide encoding the AAV capsid variant of any one of embodiments 1-109.
111. The polynucleotide of embodiment 110, which comprises:
   (i) a nucleotide sequence comprising at least one, two, three, four, five, six, or seven modifications, e.g., substitutions, but no more than ten modifications, e.g., substitutions, relative to the nucleotide sequences of SEQ ID NO: 944;
   (ii) a nucleotide sequence comprising at least one, two, three, four, five, six, or seven, but no more than ten different nucleotides, relative to the nucleotide sequences of SEQ ID NO: 944; or
   (iii) the nucleotide sequence of SEQ ID NO: 944, or nucleotide sequence substantially identical (e.g., having at least 70%, 75%, 80%, 85%, 90%, 92%, 95%, 97%, 98%, or 99% sequence identity) thereto.
112. The polynucleotide of embodiment 110 or 111, which comprises the nucleotide sequence of SEQ ID NO: 984, or a nucleotide sequence with at least 80% (e.g., at least about 85, 90, 95, 96, 97, 98, or 99%) sequence identity thereto.
113. A polynucleotide encoding an AAV capsid variant, wherein the polynucleotide comprises the nucleotide sequence of SEQ ID NO: 984.
114. A polynucleotide encoding an AAV capsid variant, wherein the encoded AAV capsid variant comprise the amino acid sequence of SEQ ID NO: 982.
115. A polynucleotide encoding an AAV capsid variant, wherein the encoded AAV capsid variant comprises:
   (a) the amino acid sequence of SEQ ID NO: 943;
   (b) an amino acid sequence comprising at least 3, 4, or 5 consecutive amino acids from SEQ ID NO: 943;
   (c) an amino acid sequence comprising one, two, or three but no more than four different amino acids, relative to the amino acid sequence of SEQ ID NO: 943; or
   (d) an amino sequence comprising one, two, or three but no more than four modifications, e.g., substitutions (e.g., conservative substitutions), insertions, or deletions, relative to the amino acid sequence of SEQ ID NO: 943.
116. A polynucleotide encoding an AAV capsid variant, wherein the encoded AAV capsid variant comprises:
   (i) one, two, three, four, or all of an amino acid other than T at position 578, an amino acid other than P at position 580, an amino acid other than A at position 581, an amino acid other than T at position 582, and/or an amino acid other than G at position 583, numbered according to SEQ ID NO: 138;
   (ii) an amino acid other than T at position 578, an amino acid other than P at position 580, an amino acid other than A at position 581, an amino acid other than T at position 582, and an amino acid other than G at position 583, numbered according to SEQ ID NO: 138;
   (iii) one, two, three, four, five, or all of the amino acid N at position 578, A at position 579, A at position 580, Q at position 581, A at position 582, and/or Y at position 583, numbered according to SEQ ID NO: 138;
   (iv) the amino acid N at position 578, A at position 579, A at position 580, Q at position 581, A at position 582, and/or Y at position 583, numbered according to SEQ ID NO: 138;
   (v) one, two, three, four, or all of the amino acid N at position 578, A at position 580, Q at position 581, A at position 582, and/or Y at position 583, numbered according to SEQ ID NO: 138;
   (vi) the amino acid N at position 578, A at position 580, Q at position 581, A at position 582, and/or Y at position 583, numbered according to SEQ ID NO: 138;
   (vii) one, two, three, four, or all of the amino acid substitutions T578N, P580A, A581Q, T582A, and/or G583Y, numbered according to SEQ ID NO: 138; and/or
   (viii) the amino acid substitutions T578N, P580A, A581Q, T582A, and G583Y, numbered according to SEQ ID NO: 138.
117. A polynucleotide encoding an AAV capsid variant comprising:
   (i) the amino acid sequence AQAY (SEQ ID NO: 1), optionally wherein the amino acid sequence corresponds to positions 580-583 of SEQ ID NO: 982; and an amino acid other than T at position 578, numbered according to SEQ ID NO: 138; and/or
   (ii) the amino acid sequence AQAY (SEQ ID NO: 1), optionally wherein the amino acid sequence corresponds to positions 580-583 of SEQ ID NO: 982; and the amino acid N at position 578, numbered according to SEQ ID NO: 138.
118. The polynucleotide of any one of embodiments 115-117, which comprises:
   (i) the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto;
   (ii) a nucleotide sequence comprising at least one, two, three, four, five, six, or seven, but no more than ten different nucleotides, relative to the nucleotide sequence of SEQ ID NO: 944; or
   (iii) a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 944.
119. The polynucleotide of any one of embodiments 115-118, wherein the AAV capsid variant comprises:
   (i) the amino acid sequence of SEQ ID NO: 982, or an amino acid sequence with at least 80% (e.g., at least about 85, 90, 95, 96, 97, 98, or 99%) sequence identity thereto;
   (ii) an amino acid sequence comprising one, two or three, but no more than four different amino acids, relative to the amino acid sequence of SEQ ID NO: 982; or
   (iii) an amino acid sequence comprising one, two or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 982.
120. The polynucleotide of any one of embodiments 115-119, comprising the nucleotide sequence of SEQ ID NO: 984, or a nucleotide sequence with at least 80% (e.g., at least about 85, 90, 95, 96, 97, 98, or 99%) sequence identity thereto.
121. The polynucleotide of any one of embodiments 115-120, which comprises a nucleotide sequence that is codon optimized.
122. A peptide comprising:
   (a) the amino acid sequence of SEQ ID NO: 943;
   (b) an amino acid sequence comprising at least 3, 4, or 5 consecutive amino acids from SEQ ID NO: 943;
   (c) an amino acid sequence comprising one, two, or three but no more than four different amino acids, relative to the amino acid sequence of SEQ ID NO: 943; or
   (d) an amino sequence comprising one, two, or three but no more than four modifications, e.g., substitutions (e.g., conservative substitutions), insertions, or deletions, relative to the amino acid sequence of SEQ ID NO: 943.
123. A peptide comprising the amino acid sequence of NAAQAY (SEQ ID NO: 943).
124. A peptide encoded by:
   (i) the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto; or
   (ii) a nucleotide sequence comprising at least one, two, three, four, five, six, or seven, but no more than ten different nucleotides, relative to the nucleotide sequence of SEQ ID NO: 944;
   (iii) a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 944.
125. A peptide wherein the nucleotide sequence encoding the peptide comprises:
   (i) the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto;
   (ii) a nucleotide sequence comprising at least one, two, three, four, five, six, or seven, but no more than ten different nucleotides relative to the nucleotide sequence of SEQ ID NO: 944; or
   (iii) a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 944.
126. An AAV capsid variant comprising the peptide of any one of embodiments 122-123.
127. An AAV capsid variant encoded by the polynucleotide of any one of embodiments 110-121.
128. An AAV particle comprising the AAV capsid variant of any one of embodiments 1-179, 126 or 127, an AAV capsid variant encoded by the polynucleotide of any one of embodiments 110-121, or an AAV capsid variant comprising the peptide of any one of embodiments 122-125.
129. The AAV particle of embodiment 128, which comprises a nucleotide sequence encoding a payload.
130. The AAV particle of embodiment 129, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
131. The AAV particle of embodiment 130, wherein the therapeutic protein or functional variant thereof, e.g., a recombinant protein, is associated with (e.g., aberrantly expressed in) a neurological or neurodegenerative disorder, a muscular, a muscular dystrophy, a neuromuscular disorder, or a neuro-oncological disorder.
132. The AAV particle of embodiment 130 or 131, the therapeutic protein or functional variant thereof is chosen from apolipoprotein E (APOE) (e.g., ApoE2, ApoE3 and/or ApoE4); human survival of motor neuron (SMN) 1 or SMN2; glucocerebrosidase (GBA1); aromatic L-amino acid decarboxylase (AADC); aspartoacylase (ASPA); tripeptidyl peptidase I (CLN2); beta-galactosidase (GLB1); N-sulphoglucosamine sulphohydrolase (SGSH); N-acetyl-alpha-glucosaminidase (NAGLU); iduronate 2-sulfatase (IDS); intracellular cholesterol transporter (NPC1); gigaxonin (GAN); titn; myotubularin; calpain-3 (CAPN-3); dysferlin (DYSF); gamma-sarcoglycan (SGCG); alpha-sarcoglycan (SGCA); microdystrophin; dystrophin; beta-sarcoglycan (SGCB); fukutin-related protein (FKRP); anoctamin-5 (ANO5); or a combination thereof.
133. The AAV particle of embodiment 130, wherein the antibody or antibody binding fragment binds to:
   (i) a CNS related target, e.g., an antigen associated with a neurological or neurodegenerative disorder, e.g., β-amyloid, APOE, tau, SOD1, TDP-43, huntingtin (HTT), and/or synuclein;
   (ii) a muscular or neuromuscular related target, e.g., an antigen associated with a muscular or neuromuscular disorder; or
   (iii) a neuro-oncology related target, e.g., an antigen associated with a neuro-oncological disorder, e.g., HER2, or EGFR (e.g., EGFRvIII).
134. The AAV particle of embodiment 130, wherein the enzyme comprises a meganuclease, a zinc finger nuclease, a TALEN, a recombinase, integrase, a base editor, a Cas9, or a fragment thereof.
135. The AAV particle of embodiment 130, wherein the component of a gene editing system comprises one or more components of a CRISPR-Cas system.
136. The AAV particle of embodiment 135, wherein the one or more components of the CRISPR-Cas system comprises a Cas9, e.g., a Cas9 ortholog or a Cpf1, and a single guide RNA (sgRNA), optionally wherein:
   (i) the sgRNA is located upstream (5′) of the Cas9 enzyme; or
   (ii) the sgRNA is located downstream (3′) of the Cas9 enzyme.
137. The AAV particle of embodiment 130, wherein the RNAi agent (e.g., a dsRNA, siRNA, shRNA, pre-miRNA, pri-miRNA, miRNA, stRNA, lncRNA, piRNA, or snoRNA), modulates, e.g., inhibits, expression of, a CNS related gene, mRNA, and/or protein.
138. The AAV particle of embodiment 137, wherein the CNS related gene is chosen from SOD1, MAPT, APOE, HTT, C9ORF72, TDP-43, APP, BACE, SNCA, ATXN1, ATXN3, ATXN7, SCNIA-SCN5A, SCN8A-SCN11A, or a combination thereof.
139. The AAV particle of any one of embodiments 128-138, which comprises a viral genome comprising a promoter operably linked to the nucleic acid sequence encoding the payload.
140. The AAV particle of embodiment 139, wherein the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor β-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light chain (NFL) or neurofilament heavy chain (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., aMHC, cTnT, and CMV-MLC2k), a liver promoter (e.g., hAAT, TBG), a skeletal muscle promoter (e.g., desmin, MCK, C512) or a functional fragment, e.g., a truncation, or a functional variant thereof.
141. The AAV particle of embodiment 139 or 140, wherein the promoter is an EF-1a promoter variant, e.g., a truncated EF-1a promoter.
142. The AAV particle of any one of embodiments 139-141, wherein the promoter comprises the nucleotide sequence of any one of SEQ ID NOs: 2100, 2101, 2103, 2104, 2108, 2109, or 2111-2120, or a nucleotide sequence provided in Table 8, a nucleotide sequence comprising at least one, two, three, four, five, six, or seven but no more than ten modifications, e.g., substitutions, insertions, or deletions, relative to the nucleotide sequence of SEQ ID NOs: 2100, 2101, 2103, 2104, 2108, 2109, or 2111-2120, or a nucleotide sequence provided in Table 8, or a nucleotide sequence with at least 80% (e.g., 85%, 90%, 95%, 96%, 97%, 98%, or 99%) sequence identity to any one of SEQ ID NOs: 2100, 2101, 2103, 2104, 2108, 2109, or 2111-2120, or a nucleotide sequence provided in Table 8.
143. The AAV particle of any one of embodiments 139-142 wherein the viral genome further comprises a polyA signal sequence.
144. The AAV particle of any one of embodiments 139-143, wherein the viral genome further comprises an inverted terminal repeat (ITR) sequence.
145. The AAV particle of any one of embodiments 139-144, wherein the viral genome comprises an ITR sequence positioned 5′ relative to the nucleic acid sequence encoding the payload.
146. The AAV particle of any one of embodiments 139-145, wherein the viral genome comprises an ITR sequence positioned 3′ relative to the nucleic acid sequence encoding the payload.
147. The AAV particle of any one of embodiments 139-146, wherein the viral genome comprises an ITR sequence positioned 5′ relative to the payload and an ITR sequence positioned 3′ relative to the nucleic acid sequence encoding the payload.
148. The AAV particle of any one of embodiments 139-147, wherein the viral genome further comprises an enhancer, a Kozak sequence, an intron region, and/or an exon region.
149. The AAV particle of any one of embodiments 139-147, wherein the viral genome further comprises a nucleotide sequence encoding a miR binding site, e.g., a miR binding site that modulates, e.g., reduces, expression of payload encoded by the viral genome in a cell or tissue where the corresponding miRNA is expressed.
150. The AAV particle of embodiment 149, wherein the encoded miRNA binding site is complementary, e.g., fully complementary or partially complementary, to a miRNA expressed in a cell or tissue of the DRG, liver, heart, hematopoietic, or a combination thereof.
151. The AAV particle of embodiment 149 or 150, wherein the encoded miR binding site modulates, e.g., reduces, expression of the encoded antibody molecule in a cell or tissue of the DRG, liver, heart, hematopoietic lineage, or a combination thereof.
152. The AAV particle of any one of embodiments 139-151, wherein the viral genome comprises at least 1-5 copies of the encoded miR binding site, e.g., at least 1, 2, 3, 4, or 5 copies.
153. The AAV particle of any one of embodiments 139-152, wherein the viral genome comprises at least 3 copies of an encoded miR binding sites, optionally wherein all three copies comprise the same miR binding site, or at least one, two, three, or all of the copies comprise a different miR binding site.
154. The AAV particle of embodiment 153, wherein the 3 copies of the encoded miR binding sites are continuous (e.g., not separated by a spacer).
155. The AAV particle of embodiment 153, wherein the 3 copies of the encoded miR binding sites are separated by a spacer, optionally wherein the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to GATAGTTA.
156. The AAV particle of any one of embodiments 139-155, wherein the viral genome comprises at least 4 copies of an encoded miR binding site, optionally wherein all four copies comprise the same miR binding site, or at least one, two, three, or all of the copies comprise a different miR binding site.
157. The AAV particle of embodiment 156, wherein the 4 copies of the encoded miR binding sites are continuous (e.g., not separated by a spacer).
158. The AAV particle of embodiment 156, wherein the 4 copies of the encoded miR binding sites are separated by a spacer, optionally wherein the spacer comprises the nucleotide sequence of GATAGTTA, or a nucleotide sequence comprising at one, two, or three modifications, e.g., substitutions, insertions, or deletions, but no more than four modifications, e.g., substitutions, insertions, or deletions, relative to GATAGTTA.
159. The AAV particle of any one of embodiments 139-158, wherein the encoded miR binding site comprises a miR122 binding site, a miR183 binding site, a miR-1 binding site, a miR-142-3p, or a combination thereof, optionally wherein:
   (i) the encoded miR122 binding site comprises the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673;
   (ii) the encoded miR183 binding site comprises the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676;
   (iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4679; and/or
   (iv) the encoded miR-142-3p binding site comprises the nucleotide sequence of SEQ ID NO: 4675, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4675.
160. The AAV particle of any one of embodiments 139-159, wherein the viral genome comprises an encoded miR122 binding site.
161. The AAV particle of any one of embodiments 139-160, wherein the viral genome comprises at least 1-5 copies, e.g., 1, 2, or 3 copies of a miR122 binding site, optionally wherein each copy is continuous (e.g., not separated by a spacer), or each copy is separated by a spacer, optionally wherein the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to GATAGTTA.
162. The AAV particle of embodiment 160 or 161, wherein the encoded miR122 binding site comprises the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673.
163. The AAV particle of any one of embodiments 139-162, wherein the viral genome comprises:
   (A) (i) a first encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673;
   (ii) a first spacer comprising the nucleotide sequence of GATAGTTA, or a nucleotide sequence comprising one, two, or three modifications, e.g., substitutions, but no more than four modifications, e.g., substitutions, relative to GATAGTTA; and
   (iii) a second encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673; or
   (B) (i) a first encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673;
   (ii) a first spacer comprising the nucleotide sequence of GATAGTTA, or a nucleotide sequence comprising one, two, or three modifications, e.g., substitutions, but no more than four modifications, e.g., substitutions, relative to GATAGTTA;
   (iii) a second encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673;
   (iv) a second spacer comprising the nucleotide sequence of GATAGTTA, or a nucleotide sequence comprising one, two, or three modifications, e.g., substitutions, but no more than four modifications, e.g., substitutions, relative to GATAGTTA; and
   (v) a third encoded miR122 binding site comprising the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673.
164. The AAV particle of any one of embodiments 139-163, wherein the viral genome comprises an encoded miR183 binding site.
165. The AAV particle of any one of embodiments 139-164, wherein the viral genome comprises at least 1-5 copies, e.g., 1, 2, or 3 copies of a miR183 binding site, optionally wherein each copy is continuous (e.g., not separated by a spacer), or each copy is separated by a spacer, optionally wherein the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to GATAGTTA.
136. The AAV particle of embodiment 164 or 165, wherein the encoded miR183 binding site comprises the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673.
167. The AAV particle of any one of embodiments 139-166, wherein the viral genome comprises: (A) (i) a first encoded miR183 binding site comprising the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676;
   (ii) a first spacer comprising the nucleotide sequence of GATAGTTA, or a nucleotide sequence comprising at least one, two, or three modifications, e.g., substitutions, insertions, or deletions, but no more than four modifications, e.g., substitutions, insertions, or deletions, relative to GATAGTTA; and
   (iii) a second encoded miR183 binding site comprising the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676; or
   (B) (i) a first encoded miR183 binding site comprising the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676;
   (ii) a first spacer comprising the nucleotide sequence of GATAGTTA, or a nucleotide sequence comprising at least one, two, or three modifications, e.g., substitutions, insertions, or deletions, but no more than four modifications, e.g., substitutions, insertions, or deletions, relative to GATAGTTA;
   (iii) a second encoded miR183 binding site comprising the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676;
   (iv) a second spacer comprising the nucleotide sequence of GATAGTTA, or a nucleotide sequence comprising at least one, two, or three modifications, e.g., substitutions, insertions, or deletions, but no more than four modifications, e.g., substitutions, insertions, or deletions, relative to GATAGTTA; and
   (v) a third encoded miR183 binding site comprising the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676.
168. The AAV particle of any one of embodiments 139-167, wherein the viral genome comprises an encoded miR122 binding site and a miR-1 binding site.
169. The AAV particle of any one of embodiments 139-168, wherein the viral genome is single stranded or self-complementary.
170. The AAV particle of any one of embodiments 139-169, wherein the viral genome further comprises a nucleotide sequence encoding a Rep protein, e.g., a non-structural protein, wherein the Rep protein comprises a Rep78 protein, a Rep68, Rep52 protein, and/or a Rep40 protein (e.g., a Rep78 and a Rep52 protein).
171. The AAV particle of any one of embodiments 128-169, wherein the AAV particle further comprises a nucleotide sequence encoding a Rep protein, e.g., a non-structural protein, wherein the Rep protein comprises a Rep78 protein, a Rep68, Rep52 protein, and/or a Rep40 protein (e.g., a Rep78 and a Rep52 protein).
172. The AAV particle of embodiment 170 or 171, wherein the Rep78 protein, the Rep68 protein, the Rep52 protein, and/or the Rep40 protein are encoded by at least one Rep gene.
173. The AAV particle of any one of embodiments 139-172, wherein the viral genome further comprises a nucleotide sequence encoding the AAV capsid variant of any one of embodiments 1-79, 96, or 97.
174. The AAV particle of any one of embodiments 128-172, wherein the AAV particle further comprises a nucleotide sequence encoding the AAV capsid variant of any one of embodiments 1-79, 96, or 97.
175. The AAV capsid variant, polynucleotide, peptide, or AAV particle of any one of the preceding embodiments, which is isolated, e.g., recombinant.
176. A vector comprising a polynucleotide encoding the AAV capsid variant of any one of embodiments 1-109, 126, 127, or 175, the polynucleotide of any one of embodiments 110-121 or 175, or a polynucleotide encoding the peptide of any one of embodiments 122-125 or 175.
177. A cell, e.g., a host cell, comprising the AAV capsid variant of any one of embodiments 1-109, 126, 127, or 175, the polynucleotide of any one of embodiments 110-121 or 175, the peptide of any one of embodiments 122-125 or 175, the AAV particle of any one of embodiments 128-175, or the vector of embodiment 431.
178. The cell of embodiment 177, wherein the cell is a mammalian cell or an insect cell.
179. The cell of embodiment 177 or 178, wherein the cell is a cell of a brain region or a spinal cord region.
180. The cell of any one of embodiments 177-179, wherein the cell is a neuron, a sensory neuron, a motor neuron, an astrocyte, a glial cell, oligodendrocyte, or a muscle cell (e.g., a cell of the heart, diaphragm, or quadriceps).
181. A method of making an AAV particle, comprising
   (i) providing a host cell comprising a viral genome; and
   (ii) incubating the host cell under conditions suitable to enclose the viral genome in the AAV capsid variant of any one of embodiments 1-79, 96, 95, or 145, or an AAV capsid variant encoded by the polynucleotide of any one of embodiments 80-91 or 145;
   thereby making the AAV particle.
182. The method of embodiment 181, further comprising, prior to step (i), introducing a first nucleic acid molecule comprising the viral genome into the host cell.
183. The method of embodiment 181 or 182, wherein the host cell comprises a second nucleic acid encoding the capsid variant.
184. The method of embodiment 183, wherein the second nucleic acid molecule is introduced into the host cell prior to, concurrently with, or after the first nucleic acid molecule.
185. A pharmaceutical composition comprising the AAV particle of any one of embodiments 128-175, an AAV particle comprising the capsid variant of any one of embodiments 1-109, 126, 127, or 175, an AAV particle comprising the peptide of any one of embodiments 122-125 or 175, and a pharmaceutically acceptable excipient.
186. A method of delivering a payload to a cell or tissue (e.g., a CNS cell or a CNS tissue), comprising administering an effective amount of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175.
187. The method of embodiment 186, wherein the cell is a cell of a brain region or a spinal cord region; or the cell is a cell of a muscle, e.g., a muscle region.
188. The method of embodiment 186 or 187, wherein the cell or tissue is within a subject.
189. The method of embodiment 188, wherein the subject has, has been diagnosed with having, or is at risk of having a genetic disorder, e.g., a monogenic disorder or a polygenic disorder.
190. The method of embodiment 188 or 189, wherein the subject has, has been diagnosed with having, or is at risk of having a neurological, e.g., a neurodegenerative disorder.
191. The method of embodiment 188 or 190, wherein the subject has, has been diagnosed with having, or is at risk of having a muscular disorder, a muscular dystrophy, or a neuromuscular disorder.
192. The method of embodiment 188 or 190, wherein the subject has, has been diagnosed with having, or is at risk of having a neuro-oncological disorder.
193. A method of treating a subject having or diagnosed with having a genetic disorder, e.g., a monogenic disorder or a polygenic disorder, comprising administering to the subject an effective amount of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175.
194. A method of treating a subject having or diagnosed with having a neurological disorder, e.g., a neurodegenerative disorder, comprising administering to the subject an effective amount of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175.
195. A method of treating a subject having or diagnosed with having a muscular disorder, a muscular dystrophy, or a neuromuscular disorder, comprising administering to the subject an effective amount of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175.
196. A method of treating a subject having or diagnosed with having a neuro-oncological disorder, comprising administering to the subject an effective amount of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175.
197. The method of any one of embodiments 189-196, wherein the genetic disorder, neurological disorder, neurodegenerative disorder, muscular disorder, a muscular dystrophy, neuromuscular disorder, or neuro-oncological disorder is Duchenne muscular dystrophy (DMD), Limb-Girdle Muscular Dystrophy (LGMD2A), Becker muscular dystrophy (BMD), congenital muscular dystrophy, Facioscapulohumeral muscular dystrophy, titinopathy, Emery Dreifuss muscular dystrophy, X-linked myotubular myopathy, Huntington's Disease, Amyotrophic Lateral Sclerosis (ALS), Gaucher Disease, Dementia with Lewy Bodies, Parkinson's disease, Spinal Muscular Atrophy, Alzheimer's Disease, a leukodystrophy (e.g., Alexander disease, autosomal dominant leukodystrophy with autonomic diseases (ADLD), Canavan disease, cerebrotendinous xanthomatosis (CTX), metachromatic leukodystrophy (MLD), Pelizaeus-Merzbacher disease, or Refsum disease), or a cancer (e.g., a HER2/neu positive cancer or a glioblastoma).
198. The method of any one of embodiments 193-197, where treating comprises prevention of progression of the disease or disorder in the subject.
199. The method of any one of embodiments 188-198, wherein the subject is a human.
200. The method of any one of embodiments 193-199, wherein the AAV particle is administered to the subject intravenously, via intra-cisterna magna injection (ICM), intracerebrally, intrathecally, intracerebroventricularly, via intraparenchymal administration, or intramuscularly.
201. The method of any one of embodiments 193-200, wherein the AAV particle is administered to the subject via focused ultrasound (FUS), e.g., coupled with the intravenous administration of microbubbles (FUS-MB), or MRI-guided FUS coupled with intravenous administration.
202. The method of any one of embodiments 193-200, wherein the AAV particle is administered to the subject intravenously.
203. The method of any one of embodiments 193-202, wherein administration of the AAV particle results in a decreased presence, level, and/or activity of a gene, mRNA, protein, or combination thereof.
204. The method of any one of embodiments 193-202, wherein administration of the AAV particle results in an increased presence, level, and/or activity of a gene, mRNA, protein, or a combination thereof.
205. The pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175, for use in a method of delivering a payload to a cell or tissue.
206. The pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175, for use in a method of treating a genetic disorder, a neurological disorder, a neurodegenerative disorder, a muscular disorder, a muscular dystrophy, a neuromuscular disorder, or a neuro-oncological disorder.
207. The pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175, for use in the manufacture of a medicament.
208. Use of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175, in the manufacture of a medicament.
209. Use of the pharmaceutical composition of embodiment 185, the AAV particle of any one of embodiments 128-175, an AAV particle comprising the capsid variant of any one of embodiments 1-109, 126, 127, or 175, an AAV particle comprising the peptide of any one of embodiments 122-125 or 175, in the manufacture of a medicament for treating a genetic disorder, a neurological disorder, a neurodegenerative disorder, a muscular disorder, a muscular dystrophy, a neuromuscular disorder, or a neuro-oncological disorder.

The details of one or more embodiments of the disclosure are set forth in the accompanying description below. Other features, objects and advantages of the disclosure will be apparent from the description. In the description, the singular forms also include the plural unless the context clearly dictates otherwise. Certain terms are defined in the Definition section and throughout.

### DETAILED DESCRIPTION OF THE DISCLOSURE

Described herein, inter alia, are compositions comprising an AAV capsid variant, e.g., an AAV capsid variant described herein, and methods of making and using the same. Generally, the AAV capsid variant has enhanced tropism for a cell or tissue, e.g., for the delivery of a payload to said cell or tissue, for example, a CNS tissue, a CNS cell, a heart cell, a heart tissue, a muscle cell, a muscle tissue, a liver cell, or a liver tissue.

Without wishing to be bound by theory, certain AAV capsid variants described herein can show multiple advantages over wild-type AAV5, including (i) increased penetrance through the blood brain barrier following intravenous administration, (ii) wider distribution throughout the multiple brain regions, (iii) elevated payload expression in multiple brain regions, (iv) wider distribution in one or more peripheral tissues, e.g., a muscle tissue, and/or (v) elevated payload expression in one or more peripheral tissues, e.g., a muscle tissue. Without wishing to be being bound by theory, it is believed that these advantages may be due, in part, to the dissemination of the AAV capsid variants through the brain vasculature. In some embodiments, the AAV capsids described herein enhance the delivery of a payload to multiple regions of the brain.

In some embodiments, AAV capsid variants disclosed herein comprise a modification in loop VIII of AAV5, e.g., at positions between 578-583, e.g., at positions 578, 580, 581, 582, and 583, numbered relative to SEQ ID NO: 138. Without wishing to be bound by theory, it is believed in some embodiments that the aforesaid region (e.g., positions between 578-583, e.g., at positions 578, 580, 581, 582, and 583) of the AAV5 capsid protrudes above the 3-fold axis of symmetry, e.g., is a surface-exposed location in the AAV5 capsid, e.g., as described in Govindasamy et al. “Structural Insights into Adeno-Associated Virus Serotype 5,” Journal of Virology, 2013, 87 (20): 11187-11199 (the contents of which are hereby incorporated by reference in their entirety). In some embodiments, loop (e.g., loop VIII) is used interchangeably herein with the term variable region (e.g., variable region VIII), or VR (e.g., VR-VIII). In some embodiments loop VIII (e.g., VR-VIII) comprises positions 571-592 (e.g., amino acids TNNQSSTTAPATGTYNLQEIVP (SEQ ID NO: 4)), numbered according to SEQ ID NO: 138. In some embodiments, loop VIII or variable region VIII (VR-VIII) is as described in Govindasamy et al. (supra) (the contents of which are hereby incorporated by reference in their entirety).

Several approaches have been used to produce AAV capsids with enhanced tropism for a cell or tissue, e.g., a CNS cell or tissue. One approach used co-infection of cultured cells (Grimm et al. In vitro and in vivo gene therapy vector evolution via multispecies interbreeding and retargeting of adeno-associated viruses. J. Virol. 2008 June 82 (12): 5887-5911) or in situ animal tissue (Lisowski et al. Selection and evaluation of clinically relevant AAV variants in a xenograft liver model. Nature 2014 506:382-386) with adenovirus, in order to trigger exponential replication of infectious AAV DNA. Another approach involved the use of cell-specific CRE transgenic mice (Deverman et al. Cre-dependent selection yields AAV variants for widespread gene transfer to the adult brain. Nat Biotechnol. 2016 Feb. 34 (2) 204-209; which is herein incorporated by reference) allowing viral DNA recombination specifically in astrocytes, followed by recovery of CRE-recombined capsid variants. Other approaches apply high throughput DNA synthesis, multiplexing, sequencing technologies, and machine learning to evaluate sequencing reads of viral DNA in different tissues to engineer variant capsids. These approaches are different from the approach disclosed herein.

There are some limitations to the art-known capsid generation methods. For example, the transgenic CRE system used by Deverman et al. (2016) has limited capabilities in other animal species and AAV variants selected by directed evolution in mouse tissue do not show similar properties in large animals. Previously described transduction-specific approaches are not amenable to large animal studies because: 1) many tissues of interest (e.g., CNS) are not readily accessible to adenovirus co-infection, 2) the specific adenovirus tropism itself would bias the library distribution, and 3) large animals are typically not amenable to transgenesis or genetic engineering to express CRE recombinase in defined cell types.

To address these limitations, a broadly applicable functional AAV capsid library screening platform for cell type-specific biopanning in non-transgenic animals has been developed and is described in the appended Examples. In the TRACER (Tropism Redirection of AAV by Cell type-specific Expression of RNA) platform system, the capsid gene is placed under the control of a cell type-specific promoter to drive capsid mRNA expression in the absence of helper virus co-infection. Without wishing to be bound by theory, it is believed that this RNA-driven screen increases the selective pressure in favor of capsid variants which transduce a specific cell type. The TRACER platform allows for generation of AAV capsid libraries whereby specific recovery and subcloning of capsid mRNA expressed in transduced cells is achieved with no need for transgenic animals or helper virus co-infection. Without wishing to be bound by theory, it is believed that since mRNA transcription is a hallmark of full transduction, the methods disclosed herein allow identification of fully infectious AAV capsid mutants, and in addition to its higher stringency, this method allows identification of capsids with high tropism for particular cell types using libraries designed to express CAP mRNA under the control of any cell-specific promoter such as, but not limited to, synapsin-1 promoter (neurons), GFAP promoter (astrocytes), TBG promoter (liver), CAMK promoter (skeletal muscle), MYH6 promoter (cardiomyocytes). Described herein are AAV capsid variants generated using the TRACER method which demonstrate enhance tropism in for example a CNS cell, a CNS tissue, a muscle cell, or a muscle tissue.

The AAV particles and payloads of the disclosure may be delivered to one or more target cells, tissues, organs, or organisms. In some embodiments, the AAV particles of the disclosure demonstrate enhanced tropism for a target cell type, tissue, or organ. As a non-limiting example, the AAV particle may have enhanced tropism for cells and tissues of the central or peripheral nervous systems (CNS and PNS, respectively). In some embodiments, an AAV particle of the disclosure may, in addition, or alternatively, have decreased tropism for a cell-type, tissue, or organ.

In some embodiments, an AAV particle is used as a biological tool due to a relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

In some embodiments, the AAV particle is a naturally occurring (e.g., wild-type) AAV or a recombinant AAV. In some embodiments, the wild-type AAV viral genome is a linear, single-stranded DNA (ssDNA) molecule approximately 5,000 nucleotides (nt) in length. In some embodiments, inverted terminal repeats (ITRs) cap the viral genome at both the 5′ and the 3′ end, providing origins of replication for the viral genome. In some embodiments, an AAV viral genome typically comprises two ITR sequences. These ITRs have a characteristic T-shaped hairpin structure defined by a self-complementary region (145nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In some embodiments, the wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are used for replication and packaging, while the capsid proteins are assembled to create the protein shell of the AAV, or AAV capsid polypeptide, e.g., an AAV capsid variant. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV5 (SEQ ID NO: 138 and 137) VP1 refers to amino acids 1-724, VP2 refers to amino acids 137-724, and VP3 refers to amino acids 193-724. In some embodiments, for the amino acid sequence of SEQ ID NO: 982, VP1 comprises amino acids 1-724, VP2 comprises amino acids 137-724, and VP3 comprises amino acids 193-724. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1: VP2: VP3.

AAV particles of the present disclosure may be produced recombinantly and may be based on adeno-associated virus (AAV) reference sequences. In addition to single stranded AAV viral genomes (e.g., ssAAVs), the present disclosure also provides for self-complementary AAV (scAAVs) viral genomes. scAAV viral genomes contain DNA strands which anneal together to form double stranded DNA. By skipping second strand synthesis, scAAVs allow for rapid expression in the transduced cell. In some embodiments, the AAV particle of the present disclosure is an scAAV. In some embodiments, the AAV particle of the present disclosure is an ssAAV.

Methods for producing and/or modifying AAV particles are disclosed in the art such as pseudotyped AAV particles (PCT Patent Publication Nos. WO200028004; WO200123001; WO2004112727; WO2005005610; and WO2005072364, the content of each of which is incorporated herein by reference in its entirety).

As described herein, the AAV particles of the disclosure comprising an AAV capsid variant, and a viral genome, have enhanced tropism for a cell-type or a tissue, e.g., a CNS cell-type, region, or tissue.

### Peptides

Disclosed herein are peptides, and associated AAV particles comprising an AAV capsid variant and a peptide for enhanced or improved transduction of a target tissue (e.g., cells of the CNS or PNS). In some, embodiments, the peptide is an isolated, e.g., recombinant, peptide. In some embodiments, the nucleic acid encoding the peptide is an isolated, e.g., recombinant, nucleic acid.

In some embodiments, the peptide may increase distribution of an AAV particle to a cell, region, or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, and/or temporal), thalamus, hypothalamus, striatum, putamen, caudate nucleus, hippocampus, entorhinal cortex, basal ganglia, or deep cerebellar nuclei. In some embodiments, the tissue of the CNS is a temporal cortex, perirhinal cortex, globus pallidus, putamen, caudate, thalamus, hippocampus, geniculate nucleus, Purkinje Layer, deep cerebellar nuclei, cerebellum, cervical spinal cord, thoracic spinal cord, or lumbar spinal cord.

In some embodiments, the peptide may increase distribution of an AAV particle to a cell, region, or tissue of the PNS. The cell or tissue of the PNS may be, but is not limited to, a dorsal root ganglion (DRG).

In some embodiments, the peptide may increase distribution of an AAV particle to the CNS (e.g., the cortex) after intravenous administration. In some embodiments, the peptide may increase distribution of an AAV particle to the CNS (e.g., the cortex) following focused ultrasound (FUS), e.g., coupled with the intravenous administration of microbubbles (FUS-MB), or MRI-guided FUS coupled with intravenous administration.

In some embodiments, the peptide may increase distribution of an AAV particle to the PNS (e.g., DRG) after intravenous administration. In some embodiments, the peptide may increase distribution of an AAV particle to the PNS (e.g., DRG) following focused ultrasound (FUS), e.g., coupled with the intravenous administration of microbubbles (FUS-MB), or MRI-guided FUS coupled with intravenous administration.

A peptide may vary in length. In some embodiments, the peptide is about 3 to about 20 amino acids in length. As non-limiting examples, the peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length. In some embodiments, a peptide comprises about 6 to 12 amino acids in length, e.g., about 9 amino acids in length. In some embodiments, a peptide comprises about 7 to 11 amino acids in length, e.g., about 8 amino acids in length. In some embodiments, a peptide comprises about 5 to 10 amino acids in length, e.g., about 7 amino acids in length. In some embodiments, a peptide comprises about 4 to 9 amino acids in length, e.g., about 6 amino acids in length.

In some embodiments a peptide may comprise a sequence as set forth in Table 1 (e.g., comprising the amino acid sequence of SEQ ID NO: 943 or 744). In some embodiments, the peptide is isolated, e.g., recombinant.

TABLE 1
Exemplary Peptide Sequences

| SEQ | | SEQ | |
|---|---|---|---|
| ID | Amino Acid | ID | Nucleotide |
| NO: | Sequence | NO: | Sequence |
| 943 | NAAQAY | 944 | AATGCGGCGCAGGCGTAT |
| 744 | ATNNQSSTNAAQAYT | 745 | GCGACGAATAATCAGTCT |
| | | | TCTACGAATGCGGCGCAG |
| | | | GCGTATACG |

In some embodiments, a peptide described herein comprises an amino acid sequence comprising at least 3, 4, or 5 consecutive amino acids from SEQ ID NO: 943. In some embodiments, the 3 consecutive amino acids comprise NAA. In some embodiments, the 4 consecutive amino acids comprise NAAQ (SEQ ID NO: 2). In some embodiments, the 5 consecutive amino acids comprise NAAQA (SEQ ID NO: 3). In some embodiments, the 6 consecutive amino acids comprise NAAQAY (SEQ ID NO: 943).

In some embodiments, a peptide described herein comprises an amino acid sequence comprising one, two, or three but no more than four different amino acids, relative to the amino acid sequence of NAAQAY (SEQ ID NO: 943). In some embodiments, the peptide comprises one or two (e.g., no more than two) different amino acids, relative to the amino acid sequence of NAAQAY (SEQ ID NO: 943).

In some embodiments, a peptide described herein comprises an amino acid sequence comprising one, two, or three but no more than four modifications, e.g., substitutions (e.g., conservative substitutions), insertions, or deletions, relative to the amino acid sequence of NAAQAY (SEQ ID NO: 943). In some embodiments, the peptide comprises an amino acid sequence comprising one or two (e.g., no more than two) modifications, e.g., substitutions (e.g., conservative substitutions), insertions, or deletions, relative to the amino acid sequence of NAAQAY (SEQ ID NO: 943).

In some embodiments, the peptide comprises the amino acid sequence of any of the sequences provided in Table 1. In some embodiments, the peptide comprises the amino acid sequence of SEQ ID NOs: 943 or 744. In some embodiments, the peptide comprises the amino acid sequence of SEQ ID NO: 943. In some embodiments, the peptide comprises the amino acid sequence of SEQ ID NO: 744.

In some embodiments, the peptide comprises an amino acid sequence encoded by a nucleotide sequence described herein, e.g., a nucleotide sequence of Table 1. In some embodiments, the peptide comprises an amino acid sequence encoded by a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 944. In some embodiments, the peptide comprises an amino acid sequence encoded by a nucleotide sequence comprising at least one, two, three, four, five, six, or seven, but no more than ten different nucleotides, relative to the nucleotide sequence of SEQ ID NO: 944. In some embodiments, the peptide comprises an amino acid sequence encoded by the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto. In some embodiments, the peptide comprises an amino acid sequence encoded by a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 745. In some embodiments, the peptide comprises an amino acid sequence encoded by a nucleotide sequence comprising at least one, two, three, four, five, six, or seven, but no more than ten different nucleotides, relative to the nucleotide sequence of SEQ ID NO: 745. In some embodiments, the peptide comprises an amino acid sequence encoded by the nucleotide sequence of SEQ ID NO: 745, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding a peptide described herein comprises a nucleotide sequence described herein, e.g., as described in Table 1. In some embodiments, the nucleotide sequence encoding a peptide described herein is codon optimized. In some embodiments, the nucleotide sequence encoding a peptide described herein is isolated, e.g., recombinant.

In some embodiments, the nucleotide sequence encoding a peptide described herein comprises the nucleotide sequence of SEQ ID NO: 944, or a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 944. In some embodiments, the nucleotide sequence encoding a peptide described herein comprises a nucleotide sequence comprising at least one, two, three, four, five, six, or seven, but no more than ten different nucleotides, relative to the nucleotide sequence of SEQ ID NO: 944. In some embodiments the nucleic acid encoding a peptide described herein comprises a nucleotide sequence comprising the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto.

In some embodiments, the nucleotide sequence encoding a peptide described herein comprises the nucleotide sequence of SEQ ID NO: 745, or a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 745. In some embodiments, the nucleotide sequence encoding a peptide described herein comprises a nucleotide sequence comprising at least one, two, three, four, five, six, or seven, but no more than ten different nucleotides, relative to the nucleotide sequence of SEQ ID NO: 745. In some embodiments the nucleic acid encoding a peptide described herein comprises a nucleotide sequence comprising the nucleotide sequence of SEQ ID NO: 745, or a nucleotide sequence substantially identical (e.g., having at least 70%, 75%, 80%, 85%, 90%, 92%, 95%, 97%, 98%, or 99% sequence identity) thereto.

The present disclosure also provides a nucleic acid or polynucleotide encoding any of the peptides described herein and AAV capsid variants, AAV particles, vectors, and cells comprising the same.

### AAV Capsid Variant

In some embodiments, an AAV particle described herein comprises an AAV capsid variant, e.g., an AAV capsid variant described herein (e.g., an AAV capsid variant comprising a peptide or an amino acid sequence described herein). In some embodiments, an AAV capsid variant comprises a peptide as set forth in Tables 1 or 9.

In some embodiments, an AAV capsid variant described herein comprises an amino acid sequence comprising at least 3, 4, or 5 consecutive amino acids from SEQ ID NO: 943. In some embodiments, the amino acid sequence is present in loop VIII. In some embodiments, the amino acid sequence replaces one, two, three, four, five, or all of positions 578, 579, 580, 581, 582, and/or 583 (e.g., positions T578, A579, P580, A581, T582, and/or G583), relative to a reference sequence numbered according to the amino acid sequence of SEQ ID NO: 138. In some embodiments, the AAV capsid variant comprises one or more amino acid substitutions at positions 578, 579, 580, 581, 582, 583 (e.g., positions T578, A579, P580, A581, T582, and/or G583), relative to a reference sequence numbered according to the amino acid sequence of SEQ ID NO: 138.

In some embodiments, the 3 consecutive amino acids comprise NAA. In some embodiments, the 4 consecutive amino acids comprise NAAQ (SEQ ID NO: 2). In some embodiments, the 5 consecutive amino acids comprise NAAQA (SEQ ID NO: 3). In some embodiments, the 6 consecutive amino acids comprise NAAQAY (SEQ ID NO: 943).

In some embodiments, an AAV capsid variant described herein comprises an amino acid sequence comprising one, two, or three but no more than four modifications, e.g., substitutions (e.g., conservative substitutions), insertions, or deletions, relative to the amino acid sequence of any one of the sequences provided in Table 1. In some embodiments, the AAV capsid variant comprises amino acid sequence comprising one or two (e.g., no more than two) modifications, e.g., substitutions (e.g., conservative substitutions), insertions, or deletions, relative to the amino acid sequence of any one of the sequences provided in Table 1. In some embodiments, the AAV capsid variant comprises an amino acid sequence comprising one, two, or three but no more than four different amino acids, relative to the amino acid sequence of any one of the sequences provided in Table 1. In some embodiments, the AAV capsid variant comprises an amino acid sequence comprising one or two (e.g., no more than two) different amino acids, relative to the amino acid sequence of any one of the sequences provided in Table 1.

In some embodiments, the AAV capsid variant comprises an amino acid sequence comprising one, two, or three but no more than four modifications, e.g., substitutions (e.g., conservative substitutions), insertions, or deletions, relative to the amino acid sequence of NAAQAY (SEQ ID NO: 943). In some embodiments, the peptide comprises an amino acid sequence comprising one or two (e.g., no more than two) modifications, e.g., substitutions (e.g., conservative substitutions), insertions, or deletions, relative to the amino acid sequence of NAAQAY (SEQ ID NO: 943). In some embodiments, the AAV capsid variant comprises an amino acid sequence comprising one, two, or three but no more than four different amino acids, relative to the amino acid sequence of NAAQAY (SEQ ID NO: 943). In some embodiments, the peptide comprises one or two (e.g., no more than two) different amino acids, relative to the amino acid sequence of NAAQAY (SEQ ID NO: 943). In some embodiments, the amino acid sequence is present in loop VIII. In some embodiments, the amino acid sequence replaces one, two, three, four, five, or all of positions 578, 579, 580, 581, 582, and/or 583 (e.g., positions T578, A579, P580, A581, T582, and/or G583), relative to a reference sequence numbered according to the amino acid sequence of SEQ ID NO: 138. In some embodiments, the amino acid sequence replaces positions 578, 579, 580, 581, 582, and 583 (e.g., positions T578, A579, P580, A581, T582, and/or G583), relative to a reference sequence numbered according to the amino acid sequence of SEQ ID NO: 138.

In some embodiments, an AAV capsid variant described herein comprises the amino acid sequence of NAAQAY (SEQ ID NO: 943), wherein the amino acid sequence replaces positions 578, 579, 580, 581, 582, and 583 (e.g., positions T578, A579, P580, A581, T582, and/or G583), relative to a reference sequence numbered according to the amino acid sequence of SEQ ID NO: 138.

In some embodiments, an AAV capsid variant described herein comprises the amino acid sequence of NAAQAY (SEQ ID NO: 943), wherein the amino acid sequence corresponds to positions 578, 579, 580, 581, 582, and 583 of SEQ ID NO: 982.

In some embodiments, an AAV capsid variant described herein comprises an amino acid other than T at position 578 (e.g., N), P at position 580 (e.g., A), A at position 581 (e.g., Q), T at position 582 (e.g., A), and/or G at position 583 (e.g., Y), numbered relative to SEQ ID NO: 138. In some embodiments, the AAV capsid variant comprises an N at position 578, numbered relative to SEQ ID NO: 138. In some embodiments, the AAV capsid variant comprises an A at position 580, numbered relative to SEQ ID NO: 138. In some embodiments, the AAV capsid variant comprises a Q at position 581, numbered relative to SEQ ID NO: 138. In some embodiments, the AAV capsid variant comprises an A at position 582, numbered relative to SEQ ID NO: 138. In some embodiments, the AAV capsid variant comprises a Y at position 583, numbered relative to SEQ ID NO: 138. In some embodiments, the AAV capsid variant corresponds to positions 578-583 of SEQ ID NO: 982.

In some embodiments, an AAV capsid variant described herein comprises one, two, three, four, or all of an amino acid other than T at position 578, an amino acid other than P at position 580, an amino acid other than A at position 581, an amino acid other than T at position 582, and/or an amino acid other than G at position 583, numbered according to SEQ ID NO: 138. In some embodiments, the AAV capsid variant comprises an amino acid other than T at position 578, an amino acid other than P at position 580, an amino acid other than A at position 581, an amino acid other than T at position 582, and an amino acid other than G at position 583, numbered according to SEQ ID NO: 138.

In some embodiments, an AAV capsid variant described herein comprises one, two, three, four, five, or all of the amino acid N at position 578, A at position 579, A at position 580, Q at position 581, A at position 582, and/or Y at position 583, numbered according to SEQ ID NO: 138. In some embodiments, the AAV capsid variant comprises the amino acid N at position 578, A at position 579, A at position 580, Q at position 581, A at position 582, and Y at position 583, numbered according to SEQ ID NO: 138.

In some embodiments, an AAV capsid variant described herein comprises one, two, three, four, or all of the amino acid N at position 578, A at position 580, Q at position 581, A at position 582, and/or Y at position 583, numbered according to SEQ ID NO: 138. In some embodiments, the AAV capsid variant comprises the amino acid N at position 578, A at position 580, Q at position 581, A at position 582, and Y at position 583, numbered according to SEQ ID NO: 138.

In some embodiments, an AAV capsid variant described herein comprises one, two, three, four, or all of the amino acid substitutions T578N, P580A, A581Q, T582A, and/or G583Y, numbered according to SEQ ID NO: 138. In some embodiments, the AAV capsid variant comprises the amino acid substitutions T578N, P580A, A581Q, T582A, and G583Y, numbered according to SEQ ID NO: 138.

In some embodiments, an AAV capsid variant described herein comprises (i) the amino acid sequence AQAY (SEQ ID NO: 1), optionally wherein the amino acid sequence replaces positions 580-583 (e.g., P580, A581, T582, G583), numbered according to SEQ ID NO: 138; and (ii) an amino acid other than T at position 578, numbered according to SEQ ID NO: 138. In some embodiments, the AAV capsid variant comprises the amino acid N at position 578, numbered according to SEQ ID NO: 138.

In some embodiments, the AAV capsid variant (e.g., an AAV capsid variant described herein), comprises an amino acid sequence encoded by the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto. In some embodiments, the AAV capsid variant described herein, comprises an amino acid sequence encoded by the nucleotide sequence of SEQ ID NO: 944, or a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 944. In some embodiments, the AAV capsid variant comprises an amino acid sequence encoded by a nucleotide sequence comprising at least one, two, three, four, five, six, or seven, but no more than ten different nucleotides relative to the nucleotide sequence of SEQ ID NO: 944.

In some embodiments, the nucleotide sequence encoding the AAV capsid variant (e.g., an AAV capsid variant described herein), comprises the nucleotide sequence of SEQ ID NO: 944, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications, e.g., substitutions, insertions, or deletions, but no more than ten modifications, e.g., substitutions, insertions, or deletions, relative to the nucleotide sequences of SEQ ID NO: 944. In some embodiments, the nucleotide sequence encoding an AAV capsid variant described herein comprises a nucleotide sequence comprising at least one, two, three, four, five, six, or seven, but no more than ten different nucleotides relative to the nucleotide sequence of SEQ ID NO: 944.

In some embodiments, the AAV capsid variant further comprises a modification, e.g., an insertion, substitution, and/or deletion in loop I, II, IV, and/or VI. In some embodiments, loop I, II, IV, VI, and VIII can be identified as described in Govindasamy et al. Structurally Mapping the Diverse Phenotype of Adeno-Associated Virus Serotype 4. Journal of Virology. 2006 Dec. 80 (23): 11556-11570; and Govindasamy et al. Structural Insights into Adeno-Associated Virus Serotype 5. Journal of Virology. 2013 Oct. 87 (20): 11187-11199; the contents of which are each hereby incorporated by reference in their entirety.

In some embodiments, additional modifications, e.g., substitutions (e.g., conservative substitutions), insertions, and/or deletions can be introduced into an AAV capsid variant described herein at positions determined using a structural map of wild-type AAV5, e.g., a structural map described and generated by Govindasamy et al. et al. Structural Insights into Adeno-Associated Virus Serotype 5. Journal of Virology. 2013 Oct. 87 (20): 11187-11199 (the contents of which are hereby incorporated herein by reference in their entirety) or Walters et al. “Structure of Adeno-Associated Virus Serotype 5,” Journal of Virology, 2004, 78 (7): 3361-3371 (the contents of which are hereby incorporated by reference in their entirety).

In some embodiments, an AAV capsid variant described herein comprises a modification as described in Jose et al. “High-Resolution Structural Characterization of a New Adenoassociated Virus Serotype 5 Antibody Epitope toward Engineering Antibody-Resistant Recombinant Gene Delivery Vectors,” Journal of Virology, 2020, 93 (1): e01394-18; Qian et al. “Directed Evolution of AAV Serotype 5 for Increased Hepatocyte Transduction and Retained Low Humoral Seroreactivity,” Molecular Therapy: Methods and Clinical Development, 2021, 20:122-132; Afione et al. “Identification and Mutagenesis of the Adeno-Associated Virus 5 Sialic Acid Binding Region,” Journal of Virology, 2015, 89 (3): 1660-1672; and/or Wang et al. “Directed evolution of adeno-associated virus 5 capsid enables specific liver tropism,” Mol Ther Nucleic Acids, 2022, 28:293-306; the contents of each of which are hereby incorporated by reference in their entirety.

In some embodiments, the AAV capsid variant, further comprises an amino acid sequence comprising at least one, two or three modifications, e.g., substitutions (e.g., conservative substitutions), insertions, or deletions, but not more than 30, 20 or 10 modifications, e.g., substitutions (e.g., conservative substitutions), insertions, or deletions, of the amino acid sequence of SEQ ID NO: 138. In some embodiments, the AAV capsid variant, further comprises an amino acid sequence comprising at least one, two or three, but not more than 30, 20 or 10 different amino acids relative to the amino acid sequence of SEQ ID NO: 138. In some embodiments, the AAV capsid variant further comprises the amino acid sequence of SEQ ID NO: 138, or an amino acid sequence with at least 70% (e.g., at least about 80, 85, 90, 95, 96, 97, 98, or 99%) sequence identity thereto.

In some embodiments, the AAV capsid variant further comprises an amino acid sequence encoded by the nucleotide sequence of SEQ ID NO: 137, or a sequence with at least 70% (e.g., at least about 80, 85, 90, 95, 96, 97, 98, or 99%) sequence identity thereto. In some embodiments, the AAV capsid variant further comprises an amino acid sequence encoded by a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 137. In some embodiments, the AAV capsid variant further comprises an amino acid sequence encoded by a nucleotide sequence comprising at least one, two or three, but not more than 30, 20 or 10 different nucleotides, relative to the amino acid sequence of SEQ ID NO: 137.

In some embodiments, the nucleotide sequence encoding the AAV capsid variant further comprises the nucleotide sequence of SEQ ID NO: 137, or a sequence with at least 70% (e.g., at least about 80, 85, 90, 95, 96, 97, 98, or 99%) sequence identity thereto. In some embodiments, the nucleotide sequence encoding the AAV capsid variant further comprises a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 137. In some embodiments, the nucleotide sequence encoding the AAV capsid variant further comprises a nucleotide sequence comprising at least one, two or three, but not more than 30, 20 or 10 different nucleotides, relative to the amino acid sequence of SEQ ID NO: 137.

In some embodiments, an AAV capsid variant of the present disclosure comprises an amino acid sequence as described herein, e.g., an amino acid sequence of an AAV capsid variant of TTK-001, e.g., as described in Tables 3 and 4.

In some embodiments, an AAV capsid variant described herein comprises a VP1, VP2, and/or VP3 protein comprising an amino acid sequence described herein, e.g., an amino acid sequence of an AAV capsid variant of TTK-001, e.g., as described in Tables 3 and 4.

In some embodiments, an AAV capsid variant described herein comprises an amino acid sequence encoded by a nucleotide sequence as described herein, e.g., a nucleotide sequence of an AAV capsid variant of TTK-001, e.g., as described in Tables 3 and 5.

In some embodiments, a polynucleotide or nucleic acid encoding an AAV capsid variant, of the present disclosure comprises a nucleotide sequence described herein, e.g., a nucleotide sequence of an AAV capsid variant of TTK-001, e.g., as described in Tables 3 and 5.

TABLE 3
Exemplary full length capsid sequences

| | | | | DNA encoding |
|---|---|---|---|---|
| | VP1 DNA SEQ ID | VP1 Amino Acid | Peptide SEQ ID | Peptide SEQ ID |
| Name | NO: | SEQ ID NO: | NO: | NO: |
| TTK-001 | 984 | 982 | 943 | 944 |

TABLE 4
Exemplary full length capsid amino acid sequences

| | SEQ | |
|---|---|---|
| Name and | ID | |
| Annotation | NO: | Amino Acid Sequence |
| TTK-001 | 982 | MSFVDHPPDWLEEVGEGLREFLGLEAGPPKPKPNQQHQDQARGLVLPGYNYLGPGN |
| (modifications | | GLDRGEPVNRADEVAREHDISYNEQLEAGDNPYLKYNHADAEFQEKLADDTSFGGN |
| at positions | | LGKAVFQAKKRVLEPFGLVEEGAKTAPTGKRIDDHFPKRKKARTEEDSKPSTSSDA |
| 578, 580, 581, | | EAGPSGSQQLQIPAQPASSLGADTMSAGGGGPLGDNNQGADGVGNASGDWHCDSTW |
| 582, and 583, | | MGDRVVTKSTRTWVLPSYNNHQYREIKSGSVDGSNANAYFGYSTPWGYFDFNRFHS |
| numbered | | HWSPRDWQRLINNYWGFRPRSLRVKIFNIQVKEVTVQDSTTTIANNLTSTVQVFTD |
| relative to SEQ | | DDYQLPYVVGNGTEGCLPAFPPQVFTLPQYGYATLNRDNTENPTERSSFFCLEYFP |
| ID NO: 138 | | SKMLRTGNNFEFTYNFEEVPFHSSFAPSQNLFKLANPLVDQYLYRFVSTNNTGGVQ |
| underlined) | | FNKNLAGRYANTYKNWFPGPMGRTQGWNLGSGVNRASVSAFATTNRMELEGASYQV |
| | | PPQPNGMTNNLQGSNTYALENTMIFNSQPANPGTTATYLEGNMLITSESETQPVNR |
| | | VAYNVGGQMATNNQSSTNAAQAYTYNLQEIVPGSVWMERDVYLQGPIWAKIPETGA |
| | | HFHPSPAMGGFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTRYLTRPL |

TABLE 5
Exemplary full length capsid nucleic acid sequences

| | SEQ | |
|---|---|---|
| Name and | ID | |
| Annotation | NO: | NT Sequence |
| TTK-001 | 984 | ATGTCTTTTGTTGATCACCCTCCAGATTGGTTGGAAGAAGTTGGTGAAGGTCTTCG |
| (portion of | | CGAGTTTTTGGGCCTTGAAGCGGGCCCACCGAAACCAAAACCCAATCAGCAGCATC |
| sequence | | AAGATCAAGCCCGTGGTCTTGTGCTGCCTGGTTATAACTATCTCGGACCCGGAAAC |
| encoding the | | GGTCTCGATCGAGGAGAGCCTGTCAACAGGGCAGACGAGGTCGCGCGAGAGCACGA |
| amino acid | | CATCTCGTACAACGAGCAGCTTGAGGCGGGAGACAACCCCTACCTCAAGTACAACC |
| sequence of | | ACGCGGACGCCGAGTTTCAGGAGAAGCTCGCCGACGACACATCCTTCGGGGGAAAC |
| SEQ ID NO: | | CTCGGAAAGGCAGTCTTTCAGGCCAAGAAAAGGGTTCTCGAACCTTTTGGCCTGGT |
| 744 is | | TGAAGAGGGTGCTAAGACGGCCCCTACCGGAAAGCGGATAGACGACCACTTTCCAA |
| underlined) | | AAAGAAAGAAGGCTCGGACCGAAGAGGACTCCAAGCCTTCCACCTCGTCAGACGCC |
| | | GAAGCTGGACCCAGCGGATCCCAGCAGCTGCAAATCCCAGCCCAACCAGCCTCAAG |
| | | TTTGGGAGCTGATACAATGTCTGCGGGAGGTGGCGGCCCATTGGGCGACAATAACC |
| | | AAGGTGCCGATGGAGTGGGCAATGCCTCGGGAGATTGGCATTGCGATTCCACGTGG |
| | | ATGGGGGACAGAGTCGTCACCAAGTCCACCCGAACCTGGGTGCTGCCCAGCTACAA |
| | | CAACCACCAGTACCGAGAGATCAAAAGCGGCTCCGTCGACGGAAGCAACGCCAACG |
| | | CCTACTTTGGATACAGCACCCCCTGGGGGTACTTTGACTTTAACCGCTTCCACAGC |
| | | CACTGGAGCCCCCGAGACTGGCAAAGACTCATCAACAACTACTGGGGCTTCAGACC |
| | | CCGGTCCCTCAGAGTCAAAATCTTCAACATTCAAGTCAAAGAGGTCACGGTGCAGG |
| | | ACTCCACCACCACCATCGCCAACAACCTCACCTCCACCGTCCAAGTGTTTACGGAC |
| | | GACGACTACCAGCTGCCCTACGTCGTCGGCAACGGGACCGAGGGATGCCTGCCGGC |
| | | CTTCCCTCCGCAGGTCTTTACGCTGCCGCAGTACGGTTACGCGACGCTGAACCGCG |
| | | ACAACACAGAAAATCCCACCGAGAGGAGCAGCTTCTTCTGCCTAGAGTACTTTCCC |
| | | AGCAAGATGCTGAGAACGGGCAACAACTTTGAGTTTACCTACAACTTTGAGGAGGT |
| | | GCCCTTCCACTCCAGCTTCGCTCCCAGTCAGAACCTGTTCAAGCTGGCCAACCCGC |
| | | TGGTGGACCAGTACTTGTACCGCTTCGTGAGCACAAATAACACTGGCGGAGTCCAG |
| | | TTCAACAAGAACCTGGCCGGGAGATACGCCAACACCTACAAAAACTGGTTCCCGGG |
| | | GCCCATGGGCCGAACCCAGGGCTGGAACCTGGGCTCCGGGGTCAACCGCGCCAGTG |
| | | TCAGCGCCTTCGCCACGACCAATAGGATGGAGCTCGAGGGCGCGAGTTACCAGGTG |
| | | CCCCCGCAGCCGAACGGCATGACCAACAACCTCCAGGGCAGCAACACCTATGCCCT |
| | | GGAGAACACTATGATCTTCAACAGCCAGCCGGCGAACCCGGGCACCACCGCCACGT |
| | | ACCTCGAGGGCAACATGCTCATCACCAGCGAGAGCGAGACGCAGCCGGTGAACCGC |
| | | GTGGCGTACAACGTCGGCGGGCAGATGGCGACGAATAATCAGTCTTCTACGAATGC |
| | | GGCGCAGGCGTATACGTACAACCTCCAGGAAATCGTGCCCGGCAGCGTGTGGATGG |
| | | AGAGGGACGTGTACCTCCAAGGACCCATCTGGGCCAAGATCCCAGAGACGGGGGCG |
| | | CACTTTCACCCCTCTCCGGCCATGGGCGGATTCGGACTCAAACACCCACCGCCCAT |
| | | GATGCTCATCAAGAACACGCCTGTGCCCGGAAATATCACCAGCTTCTCGGACGTGC |
| | | CCGTCAGCAGCTTCATCACCCAGTACAGCACCGGGCAGGTCACCGTGGAGATGGAG |
| | | TGGGAGCTCAAGAAGGAAAACTCCAAGAGGTGGAACCCAGAGATCCAGTACACAAA |
| | | CAACTACAACGACCCCCAGTTTGTGGACTTTGCCCCGGACAGCACCGGGGAATACA |
| | | GAACCACCAGACCTATCGGAACCCGATACCTTACCCGACCCCTTTAA |

In some embodiments, an AAV capsid variant described herein comprises the amino acid sequence of SEQ ID NO: 982, or an amino acid sequence with at least 70% (e.g., at least about 75, 80, 85, 90, 95, 96, 97, 98, or 99%) sequence identity thereto. In some embodiments, an AAV capsid variant described herein comprises the amino acid sequence of SEQ ID NO: 982 or an amino acid sequence with at least 90% sequence identity thereto. In some embodiments, an AAV capsid variant described herein comprises the amino acid sequence of SEQ ID NO: 982 or an amino acid sequence with at least 95% sequence identity thereto. In some embodiments, an AAV capsid variant described herein comprises the amino acid sequence of SEQ ID NO: 982 or an amino acid sequence with at least 96% sequence identity thereto. In some embodiments, an AAV capsid variant described herein comprises the amino acid sequence of SEQ ID NO: 982 or an amino acid sequence with at least 97% sequence identity thereto. In some embodiments, an AAV capsid variant described herein comprises the amino acid sequence of SEQ ID NO: 982 or an amino acid sequence with at least 98% sequence identity thereto. In some embodiments, an AAV capsid variant described herein comprises the amino acid sequence of SEQ ID NO: 982 or an amino acid sequence with at least 99% sequence identity thereto. In some embodiments, an AAV capsid variant described herein comprises an amino acid sequence comprising at least one, two, or three modifications, e.g., substitutions (e.g., conservative substitutions), insertions, or deletions, but not more than 30, 20 or 10 modifications, e.g., substitutions (e.g., conservative substitutions), insertions, or deletions, relative to the amino acid sequence of SEQ ID NO: 982. In some embodiments, the AAV capsid variant, comprises an amino acid sequence comprising at least one, two or three, but not more than 30, 20 or 10 different amino acids, relative to the amino acid sequence of SEQ ID NO: 982.

In some embodiments, an AAV capsid variant described herein comprises an amino acid sequence encoded by the nucleotide sequence of SEQ ID NO: 984, or a nucleotide sequence with at least 70% (e.g., at least about 75, 80, 85, 90, 95, 96, 97, 98, or 99%) sequence identity thereto. In some embodiments, an AAV capsid variant described herein comprises an amino acid sequence encoded by a nucleotide sequence comprising at least one, two or three, but not more than 30, 20 or 10 different nucleotides, relative to the amino acid sequence of SEQ ID NO: 984. In some embodiments, an AAV capsid variant described herein comprises an amino acid sequence encoded by a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 984.

In some embodiments, the nucleotide sequence encoding an AAV capsid variant, described herein comprises the nucleotide sequence of SEQ ID NO: 984, or a nucleotide sequence with at least 70% (e.g., at least about 75, 80, 85, 90, 95, 96, 97, 98, or 99%) sequence identity thereto. In some embodiments, the nucleotide sequence encoding an AAV capsid variant described herein comprises the nucleotide sequence of SEQ ID NO: 984, or a nucleotide sequence with at least 70% (e.g., at least about 75, 80, 85, 90, 95, 96, 97, 98, or 99%) sequence identity thereto. In some embodiments, the nucleotide sequence encoding an AAV capsid variant described herein, comprises a nucleotide sequence comprising at least one, two or three modifications, e.g., substitutions, insertions, or deletions, but not more than 30, 20 or 10 modifications, e.g., substitutions, insertions, or deletions, relative to the nucleotide sequence of SEQ ID NO: 984. In some embodiments, the nucleotide sequence encoding an AAV capsid variant described herein, comprises a nucleotide sequence comprising at least one, two or three, but not more than 30, 20 or 10 different nucleotides, relative to the amino acid sequence of SEQ ID NO: 984. In some embodiments, the nucleic acid sequence encoding an AAV capsid variant described herein is codon optimized.

In some embodiments, an AAV capsid variant described herein comprises a VP1, VP2, VP3 protein, or a combination thereof. In some embodiments, an AAV capsid variant comprises the amino acid sequence corresponding to positions 137-724, e.g., a VP2, of SEQ ID NO: 982, or a sequence with at least 70% (e.g., at least about 75, 80, 85, 90, 95, 96, 97, 98, or 99%) sequence identity thereto. In some embodiments, the AAV capsid protein comprises the amino acid sequence corresponding to positions 193-724, e.g., a VP3, of SEQ ID NO: 982, or a sequence with at least 70% (e.g., at least about 75, 80, 85, 90, 95, 96, 97, 98, or 99%) sequence identity thereto. In some embodiments, the AAV capsid variant comprises the amino acid sequence corresponding to positions 1-724, e.g., a VP1, of SEQ ID NO: 982, or an amino acid sequence with at least 70% (e.g., at least about 75, 80, 85, 90, 95, 96, 97, 98, or 99%) sequence identity thereto.

In some embodiments, an AAV capsid variant described herein has an increased tropism for a muscle cell or tissue, relative to the tropism of a reference sequence comprising the amino acid sequence of SEQ ID NO: 138 or SEQ ID NO: 139. In some embodiments, the AAV capsid variant delivers an increased level of a payload to a muscle cell or region, optionally wherein the level of the payload is increased by at least 5, 10, 12, 15, 20, 21, or 25-fold, as compared to a reference sequence of SEQ ID NO: 138 or SEQ ID NO: 139. In some embodiments, the AAV capsid variant delivers an increased level of viral genomes to a muscle cell or region, optionally wherein the level of viral genomes is increased by at least 2, 2.5, 5, 5.5, 6, 6.5, 7, 7.5, 8, 8.5, 9, 9.5, 10, 10.5, 11, 11.5, 12, 12.5, 13, 13.5, 13.8, or 14-fold, as compared to a reference sequence of SEQ ID NO: 138 or SEQ ID NO:

139. In some embodiments, the muscle cell or region, is a heart cell or region or a quadriceps cell or region.

In some embodiments, an AAV capsid variant described herein has an increased tropism for a heart cell or tissue, relative to the tropism of a reference sequence comprising the amino acid sequence of SEQ ID NO: 138 or SEQ ID NO: 139. In some embodiments, the AAV capsid variant delivers an increased level of a payload to a heart cell or region, optionally wherein the level of the payload is increased by at least 2, 2.5, 3, 3.5, 4, 4.5 or 5-fold, as compared to a reference sequence of SEQ ID NO: 138. In some embodiments, the AAV capsid variant delivers an increased level of viral genomes to a heart cell or region, optionally wherein the level of viral genomes is increased by at least 5, 6, 7, 8, 9 or 10-fold, as compared to a reference sequence of SEQ ID NO: 138 or SEQ ID NO: 139.

In some embodiments, an AAV capsid variant described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138. In some embodiments, the AAV capsid variant transduces a brain cell or region, optionally wherein the level of transduction is at least 3, 4, 5, 6, 7, 8, 9, 10, 11, 12, 13, 14, 15, 20, 24, 25, 29, or 30-fold greater as compared to a reference sequence of SEQ ID NO: 138 or SEQ ID NO: 139. In some embodiments, the AAV capsid variant delivers an increased level of a payload to a brain cell or region, optionally wherein the level of the payload is increased by at least 3, 4, 5, 6, 7, 8, 9, 10, 11, 12, 13, or 14-fold, as compared to a reference sequence of SEQ ID NO: 138 or SEQ ID NO: 139. In some embodiments, the AAV capsid variant delivers an increased level of viral genomes to a brain cell or region, optionally wherein the level of viral genomes is increased by at least 3, 4, 5, 6, 7, 8, 9, 10, 11, 12, 13, 14, 15, 20, 24, 25, 29, or 30-fold, as compared to a reference sequence of SEQ ID NO: 138 or SEQ ID NO: 139. In some embodiments, the brain region is a caudate, motor cortex, putamen, thalamus, and/or cerebellum (e.g., the molecular and granule layer of the cerebellum).

In some embodiments, an AAV capsid variant described herein is enriched at least about 4, 4.6, 10, 20, 25, 28, 30, 40, 50, 60, 70, 80, 81, 80, 100, 101, or 110-fold in the brain compared to a reference sequence of SEQ ID NO: 138.

In some embodiments, an AAV capsid variant described herein is enriched in the brain of at least two to three species, e.g., a non-human primate and rodent (e.g., mouse) species, compared to a reference sequence of SEQ ID NO: 138. In some embodiments, an AAV capsid variant described herein is enriched at least about 4, 4.6, 10, 20, 25, 28, 30, 40, 50, 60, 70, 80, 81, 80, 100, 101, or 110-fold in the brain of at least two to three species, e.g., a non-human primate and rodent (e.g., mouse) species, compared to a reference sequence of SEQ ID NO: 138 or 982. In some embodiments, the at least two to three species are Macaca fascicularis, Chlorocebus sabaeus, Callithrix jacchus, rat and/or mouse (e.g., BALB/c mice and/or C57BL6 mice).

In some embodiments an AAV capsid variant described herein shows preferential transduction in a muscle region relative to the transduction in the liver.

In some embodiments an AAV capsid variant described herein shows preferential transduction in a heart region relative to the transduction in the liver.

In some embodiments an AAV capsid variant described herein shows preferential transduction in a brain region relative to the transduction in the liver.

In some embodiments, an AAV capsid variant described herein has decreased tropism for a liver cell or tissue, relative to the tropism of a reference sequence comprising the amino acid sequence of SEQ ID NO: 138 or SEQ ID SEQ ID NO: 139.

In some embodiments, an AAV capsid variant described herein is capable of transducing neuronal cells.

In some embodiments, an AAV capsid variant of the present disclosure is isolated, e.g., recombinant. In some embodiments, a polynucleotide encoding an AAV capsid polypeptide, e.g., an AAV capsid variant, of the present disclosure is isolated, e.g., recombinant.

Also provided herein are polynucleotide sequences encoding any of the AAV capsid variants described above and AAV particles, vectors, and cells comprising the same.

### AAV Serotypes and Capsids

In some embodiments, an AAV particle of the present disclosure may comprise a capsid protein or variant thereof any natural or recombinant AAV serotype. AAV serotypes may differ in characteristics such as, but not limited to, packaging, tropism, transduction and immunogenic profiles. While not wishing to be bound by theory, it is believed in some embodiments, that the AAV capsid protein, e.g., an AAV capsid variant, can modulate AAV particle tropism in a particular tissue.

In some embodiments, an AAV capsid variant described herein allows for blood brain barrier penetration following intravenous administration. In some embodiments, the AAV capsid variant allows for blood brain barrier penetration following intravenous administration, focused ultrasound (FUS), e.g., coupled with the intravenous administration of microbubbles (FUS-MB), or MRI-guided FUS coupled with intravenous administration. In some embodiments the AAV capsid variant allows for increased distribution to a brain region. In some embodiments, the brain region comprises a temporal cortex, perirhinal cortex, globus pallidus, putamen, caudate, thalamus, hippocampus, geniculate nucleus, Purkinje Layer, deep cerebellar nuclei, cerebellum, frontal cortex, sensory cortex, motor cortex, dentate nucleus, cerebellar cortex, cerebral cortex, brain stem, or a combination thereof. In some embodiments, the AAV capsid variant allows for preferential transduction in a brain region relative to the transduction in the dorsal root ganglia (DRG). In some embodiments, the AAV capsid variant allows for preferential transduction in a brain region relative to the transduction in the liver. In some embodiments, the AAV capsid variant allows for transduction in neuronal cells. In some embodiments, the AAV capsid variant allows for transduction in a non-neuronal cell, e.g., a glial cell (e.g., an astrocyte, an oligodendrocyte, or a combination thereof). In some embodiments, the AAV capsid variant allows for transduction in both neuronal cells and non-neuronal cell, e.g., a glial cell (e.g., an astrocyte, an oligodendrocyte, or a combination thereof).

In some embodiments, an AAV capsid variant allows for increased distribution to a spinal cord region. In some embodiments, the spinal region comprises a cervical spinal cord region, thoracic spinal cord region, and/or lumbar spinal cord region.

In some embodiments the AAV capsid variant, allows for increased distribution to a heart region.

In some embodiments, the AAV capsid variant, is suitable for intramuscular administration and/or transduction of muscle fibers. In some embodiments the AAV capsid variant, allows for increased distribution to a muscle region. In some embodiments, the muscle region comprises a heart muscle, quadriceps muscle, a diaphragm muscle region, or a combination thereof.

In some embodiments the AAV capsid variant, allows for increased distribution to a liver region.

In some embodiments, an AAV capsid described herein comprises a modification as described in Jose et al. High-Resolution Structural Characterization of a New Adenoassociated Virus Serotype 5 Antibody Epitope toward Engineering Antibody-Resistant Recombinant Gene Delivery Vectors. Journal of Virology. 2019 Jan. 93 (1): e01394-18; Qian et al. Directed Evolution of AAV Serotype 5 for Increased Hepatocyte Transduction and Retained Low Humoral Seroreactivity. Molecular Therapy: Methods & Clinical Development. 2020 Oct. 20:122-132; Afione et al. Identification and Mutagenesis of the Adeno-Associated Virus 5 Sialic Binding Region. Journal of Virology. 2015 Feb. 89 (3): 1660-1672; the contents of which are each hereby incorporated by reference in their entirety.

In some embodiments, the initiation codon for translation of the AAV VP1 capsid protein, e.g., a capsid variant, described herein may be CTG, TTG, or GTG as described in US Patent No. U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (e.g. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met-/AA-). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28 (5): 255-267; Hwang, et al. N-Terminal Acetylation of Cellular Proteins Creates Specific Degradation Signals. Science. 2010 Feb. 19. 327 (5968): 973-977; the contents of which are each incorporated herein by reference in its entirety.

According to the present disclosure, references to capsid proteins, e.g., AAV capsid variants, is not limited to either clipped (Met-/AA-) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a capsid protein or capsid polypeptide (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met-/AA-).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met-) of the 736 amino acid Met+sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1-) of the 736 amino acid AA1+sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met-/AA1-), and combinations thereof (Met+/AA1+ and Met-/AA1-).

As a non-limiting example, an AAV capsid serotype can include VP1 (Met+/AA1+), VP1 (Met-/AA1-), or a combination of VP1 (Met+/AA1+) and VP1 (Met-/AA1-). An AAV capsid serotype can also include VP3 (Met+/AA1+), VP3 (Met-/AA1-), or a combination of VP3 (Met+/AA1+) and VP3 (Met-/AA1-); and can also include similar optional combinations of VP2 (Met+/AA1) and VP2 (Met-/AA1-).

### Additional AAV Sequences

In some embodiments, the AAV capsid variant, comprises immediately subsequent to position 577, numbered relative to SEQ ID NO: 138, at least 3, 4, or 5 consecutive amino acids of any of amino acid sequence provided in Table 1. In some embodiments, the amino acid sequence replaces positions 578, 579, 580, 581, 582, and/or 583, numbered according to SEQ ID NO: 138.

In some embodiments, the AAV capsid variant, comprises a modification at position 578, 580, 581, 582, and/or 583, numbered relative to SEQ ID NO: 138 or corresponding to equivalent positions in any other AAV serotype (e.g., AAV1, AAV2, AAV3, AAV3b, AAV4, AAV6, AAV7, AAV8, AAV9, AAVrh8, AAVrh10, AAVrh32.33, AAVrh74, PHP.N, PHP.B, or an AAV serotype as provided in Table 6 of WO 2021/230987 (the contents of which are hereby incorporated by reference in their entirety)). In some embodiments, the amino acid sequence of NAAQAY (SEQ ID NO: 943) replaces positions 578, 579, 580, 581, 582, and/or 583, numbered according to the amino acid sequence of SEQ ID NO: 138, numbered according to SEQ ID NO: 138, or corresponding to equivalent positions in any other AAV serotype (e.g., AAV1, AAV2, AAV3, AAV3b, AAV4, AAV6, AAV7, AAV8, AAV9, AAVrh8, AAVrh10, AAVrh32.33, AAVrh74, PHP.N, PHP.B, or an AAV serotype as provided in Table 6 of WO 2021/230987 (the contents of which are hereby incorporated by reference in their entirety)).

In some embodiments, an AAV capsid variant described herein comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments, an AAV capsid variant described herein comprises the amino acid sequence of SEQ ID NO: 138 or an amino acid sequence having at least 90% identity thereto. In some embodiments, an AAV capsid variant described herein comprises the amino acid sequence of SEQ ID NO: 138 or an amino acid sequence having at least 95% identity thereto. In some embodiments, an AAV capsid variant described herein comprises the amino acid sequence of SEQ ID NO: 138 or an amino acid sequence having at least 96% identity thereto. In some embodiments, an AAV capsid variant described herein comprises the amino acid sequence of SEQ ID NO: 138 or an amino acid sequence having at least 97% identity thereto. In some embodiments, an AAV capsid variant described herein comprises the amino acid sequence of SEQ ID NO: 138 or an amino acid sequence having at least 98% identity thereto. In some embodiments, an AAV capsid variant described herein comprises the amino acid sequence of SEQ ID NO: 138 or an amino acid sequence having at least 99% identity thereto. In some embodiments the AAV capsid polypeptide or the AAV capsid variant, comprises an amino acid sequence comprising at least one, two, or three modifications, e.g., substitutions (e.g., conservative substitutions), but no more than 30, 20, or 10 modifications, e.g., substitutions (e.g., conservative substitutions), relative to the amino acid sequence of SEQ ID NO: 138. In some embodiments the AAV capsid polypeptide or the AAV capsid variant, comprises an amino acid sequence comprising at least one, two, or three, but no more than 30, 20, or 10 different amino acids, relative to the amino acid sequence of SEQ ID NO: 138. In some embodiments, the AAV capsid polypeptide or the AAV capsid variant, comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide or the AAV capsid variant comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto.

In some embodiments, an AAV capsid variant described herein comprises the amino acid sequence or is encoded by the nucleotide sequence of any of SEQ ID NO: 1 or 2 of U.S. Pat. No. 7,427,396; SEQ ID NO: 114 of US20030138772; SEQ ID NO: 199 of US20150315612; or SEQ ID NOs: 13, 14, 16, 17, 19, 20, 22, 23, 25, 26, 28, 29, 31, 32, 34, 35, 37, 38, 40, 41, 43, or 44 of US20160289275A1 (the contents of each are incorporated herein by reference in their entirety), or a sequence substantially identical (e.g., having at least 70%, 75%, 80%, 85%, 90%, 92%, 95%, 97%, 98%, or 99% sequence identity) thereto.

In some embodiments, the AAV capsid variant described herein comprises a modification, e.g., substitution, at position 569 (e.g., M569V), 652 (e.g., D652A), 362 (e.g., T362M), 359 (e.g., Q359D), 350 (e.g., E350Q), 533 (e.g., P533S), 585 (e.g., Y585V), 587 (e.g., L587T), 581 (e.g., A581T), 582 (e.g., T582A), 584 (e.g., T584A), or a combination thereof, all numbered relative to SEQ ID NO: 138.

In some embodiments, an AAV capsid variant described herein comprises an amino acid from a wild-type AAV5 sequence, e.g., the amino acid sequence of SEQ ID NO: 138, at one or more of positions 581 to 589, numbered relative to SEQ ID NO: 138. In some embodiments, the AAV capsid variant comprises 1, 2, 3, 4, 5, 6, 7, 8, or all of: the amino acid from a wild-type AAV5 sequence (e.g., SEQ ID NO: 138) at position 581 (e.g., comprises the amino acid A at position 581); the amino acid from a wild-type AAV5 sequence (e.g., SEQ ID NO: 138) at position 582 (e.g., comprises the amino acid T at position 582); the amino acid from a wild-type AAV5 sequence (e.g., SEQ ID NO: 138) at position 583 (e.g., comprises the amino acid G at position 583); the amino acid from a wild-type AAV5 sequence (e.g., SEQ ID NO: 138) at position 584 (e.g., comprises the amino acid T at position 584); the amino acid from a wild-type AAV5 sequence (e.g., SEQ ID NO: 138) at position 585 (e.g., comprises the amino acid Y at position 585); the amino acid from a wild-type AAV5 sequence (e.g., SEQ ID NO: 138) at position 586 (e.g., comprises the amino acid N at position 586); the amino acid from a wild-type AAV5 sequence (e.g., SEQ ID NO: 138) at position 587 (e.g., comprises the amino acid L at position 587); the amino acid from a wild-type AAV5 sequence (e.g., SEQ ID NO: 138) at position 588 (e.g., comprises the amino acid Q at position 588); and/or the amino acid from a wild-type AAV5 sequence (e.g., SEQ ID NO: 138) at position 589 (e.g., comprises the amino acid E at position 589).

In certain embodiments, an AAV capsid described herein does not comprise a T at position 581, an A at position 582, an A at position 584, a V at position 585, a T at position 585, a V at position 569, an A at position 652, an M at position 362, a Q at position 359, a Q at position 350, an S at position 533, or a combination thereof, all numbered relative to SEQ ID NO: 138.

In some embodiments, an AAV capsid described herein does not comprise a modification, e.g., substitution, at positions 581-589 (numbered according to SEQ ID NO: 138), wherein the modification has the amino acid sequence of any of the sequences provided in Tables 2, 7, 8, 10, 12, 14, 16, 18, 20, 22, 24, 26, 28, 30, 32, 34, 36, 39, 41, 43, 45, 47, 49, 51, 53, 55, 57, 59, 61, 63, 65, 67, 69, or 71-86 of WO 2021/242909.

In any of the embodiments described herein, a position numbered relative to SEQ ID NO: 138 can be identified by providing an alignment of a reference sequence and a query sequence, wherein the reference sequence is SEQ ID NO: 138, and identifying the residues corresponding to the positions in the query sequence that correspond to positions in the reference sequence.

TABLE 6
AAV Sequences

| | SEQ | |
|---|---|---|
| | ID | |
| Serotype | NO: | Sequence |
| AAV5 | 138 | MSFVDHPPDWLEEVGEGLREFLGLEAGPPKPKPNQQHQDQARGLVLPGYNYLGPGNGLDR |
| WT | | GEPVNRADEVAREHDISYNEQLEAGDNPYLKYNHADAEFQEKLADDTSFGGNLGKAVFQA |
| (amino | | KKRVLEPFGLVEEGAKTAPTGKRIDDHFPKRKKARTEEDSKPSTSSDAEAGPSGSQQLQI |
| acid) | | PAQPASSLGADTMSAGGGGPLGDNNQGADGVGNASGDWHCDSTWMGDRVVTKSTRTWVLP |
| | | SYNNHQYREIKSGSVDGSNANAYFGYSTPWGYFDFNRFHSHWSPRDWQRLINNYWGFRPR |
| | | SLRVKIFNIQVKEVTVQDSTTTIANNLTSTVQVFTDDDYQLPYVVGNGTEGCLPAFPPQV |
| | | FTLPQYGYATLNRDNTENPTERSSFFCLEYFPSKMLRTGNNFEFTYNFEEVPFHSSFAPS |
| | | QNLFKLANPLVDQYLYRFVSTNNTGGVQFNKNLAGRYANTYKNWFPGPMGRTQGWNLGSG |
| | | VNRASVSAFATTNRMELEGASYQVPPQPNGMTNNLQGSNTYALENTMIFNSQPANPGTTA |
| | | TYLEGNMLITSESETQPVNRVAYNVGGQMATNNQSSTTAPATGTYNLQEIVPGSVWMERD |
| | | VYLOGPIWAKIPETGAHFHPSPAMGGFGLKHPPPMMLIKNTPVPGNITSFSDVPVSSFIT |
| | | QYSTGQVTVEMEWELKKENSKRWNPEIQYTNNYNDPQFVDFAPDSTGEYRTTRPIGTRYL |
| | | TRPL |
| AAV5 | 137 | ATGTCTTTTGTTGATCACCCTCCAGATTGGTTGGAAGAAGTTGGTGAAGGTCTTCGCGAG |
| WT | | TTTTTGGGCCTTGAAGCGGGCCCACCGAAACCAAAACCCAATCAGCAGCATCAAGATCAA |
| (DNA) | | GCCCGTGGTCTTGTGCTGCCTGGTTATAACTATCTCGGACCCGGAAACGGTCTCGATCGA |
| | | GGAGAGCCTGTCAACAGGGCAGACGAGGTCGCGCGAGAGCACGACATCTCGTACAACGAG |
| | | CAGCTTGAGGCGGGAGACAACCCCTACCTCAAGTACAACCACGCGGACGCCGAGTTTCAG |
| | | GAGAAGCTCGCCGACGACACATCCTTCGGGGGAAACCTCGGAAAGGCAGTCTTTCAGGCC |
| | | AAGAAAAGGGTTCTCGAACCTTTTGGCCTGGTTGAAGAGGGTGCTAAGACGGCCCCTACC |
| | | GGAAAGCGGATAGACGACCACTTTCCAAAAAGAAAGAAGGCTCGGACCGAAGAGGACTCC |
| | | AAGCCTTCCACCTCGTCAGACGCCGAAGCTGGACCCAGCGGATCCCAGCAGCTGCAAATC |
| | | CCAGCCCAACCAGCCTCAAGTTTGGGAGCTGATACAATGTCTGCGGGAGGTGGCGGCCCA |
| | | TTGGGCGACAATAACCAAGGTGCCGATGGAGTGGGCAATGCCTCGGGAGATTGGCATTGC |
| | | GATTCCACGTGGATGGGGGACAGAGTCGTCACCAAGTCCACCCGAACCTGGGTGCTGCCC |
| | | AGCTACAACAACCACCAGTACCGAGAGATCAAAAGCGGCTCCGTCGACGGAAGCAACGCC |
| | | AACGCCTACTTTGGATACAGCACCCCCTGGGGGTACTTTGACTTTAACCGCTTCCACAGC |
| | | CACTGGAGCCCCCGAGACTGGCAAAGACTCATCAACAACTACTGGGGCTTCAGACCCCGG |
| | | TCCCTCAGAGTCAAAATCTTCAACATTCAAGTCAAAGAGGTCACGGTGCAGGACTCCACC |
| | | ACCACCATCGCCAACAACCTCACCTCCACCGTCCAAGTGTTTACGGACGACGACTACCAG |
| | | CTGCCCTACGTCGTCGGCAACGGGACCGAGGGATGCCTGCCGGCCTTCCCTCCGCAGGTC |
| | | TTTACGCTGCCGCAGTACGGTTACGCGACGCTGAACCGCGACAACACAGAAAATCCCACC |
| | | GAGAGGAGCAGCTTCTTCTGCCTAGAGTACTTTCCCAGCAAGATGCTGAGAACGGGCAAC |
| | | AACTTTGAGTTTACCTACAACTTTGAGGAGGTGCCCTTCCACTCCAGCTTCGCTCCCAGT |
| | | CAGAACCTGTTCAAGCTGGCCAACCCGCTGGTGGACCAGTACTTGTACCGCTTCGTGAGC |
| | | ACAAATAACACTGGCGGAGTCCAGTTCAACAAGAACCTGGCCGGGAGATACGCCAACACC |
| | | TACAAAAACTGGTTCCCGGGGCCCATGGGCCGAACCCAGGGCTGGAACCTGGGCTCCGGG |
| | | GTCAACCGCGCCAGTGTCAGCGCCTTCGCCACGACCAATAGGATGGAGCTCGAGGGCGCG |
| | | AGTTACCAGGTGCCCCCGCAGCCGAACGGCATGACCAACAACCTCCAGGGCAGCAACACC |
| | | TATGCCCTGGAGAACACTATGATCTTCAACAGCCAGCCGGCGAACCCGGGCACCACCGCC |
| | | ACGTACCTCGAGGGCAACATGCTCATCACCAGCGAGAGCGAGACGCAGCCGGTGAACCGC |
| | | GTGGCGTACAACGTCGGCGGGCAGATGGCCACCAACAACCAGAGCTCTACTACTGCCCCC |
| | | GCGACCGGCACGTACAACCTCCAGGAAATCGTGCCCGGCAGCGTGTGGATGGAGAGGGAC |
| | | GTGTACCTCCAAGGACCCATCTGGGCCAAGATCCCAGAGACGGGGGCGCACTTTCACCCC |
| | | TCTCCGGCCATGGGCGGATTCGGACTCAAACACCCACCGCCCATGATGCTCATCAAGAAC |
| | | ACGCCTGTGCCCGGAAATATCACCAGCTTCTCGGACGTGCCCGTCAGCAGCTTCATCACC |
| | | CAGTACAGCACCGGGCAGGTCACCGTGGAGATGGAGTGGGAGCTCAAGAAGGAAAACTCC |
| | | AAGAGGTGGAACCCAGAGATCCAGTACACAAACAACTACAACGACCCCCAGTTTGTGGAC |
| | | TTTGCCCCGGACAGCACCGGGGAATACAGAACCACCAGACCTATCGGAACCCGATACCTT |
| | | ACCCGACCCCTTTAA |
| AAV9/ | 139 | MAADGYLPDWLEDNLSEGIREWWALKPGAPQPKANQQHQDNARGLVLPGYKYLGPGNGLD |
| hu.14 WT | | KGEPVNAADAAALEHDKAYDQQLKAGDNPYLKYNHADAEFQERLKEDTSFGGNLGRAVFQ |
| (amino | | AKKRLLEPLGLVEEAAKTAPGKKRPVEQSPQEPDSSAGIGKSGAQPAKKRLNFGQTGDTE |
| acid) | | SVPDPQPIGEPPAAPSGVGSLTMASGGGAPVADNNEGADGVGSSSGNWHCDSQWLGDRVI |
| | | TTSTRTWALPTYNNHLYKQISNSTSGGSSNDNAYFGYSTPWGYFDFNRFHCHFSPRDWQR |
| | | LINNNWGFRPKRLNFKLFNIQVKEVTDNNGVKTIANNLTSTVQVFTDSDYQLPYVLGSAH |
| | | EGCLPPFPADVEMIPQYGYLTLNDGSQAVGRSSFYCLEYFPSQMLRTGNNFQFSYEFENV |
| | | PFHSSYAHSQSLDRLMNPLIDQYLYYLSKTINGSGONQQTLKFSVAGPSNMAVQGRNYIP |
| | | GPSYRQQRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAQAQTGWVQNQG |
| | | ILPGMVWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAVNTEGV |
| | | YSEPRPIGTRYLTRNL |

Viral Genome of the AAV particle

In some embodiments, an AAV particle as described herein comprising an AAV capsid variant described herein, may be used for the delivery of a viral genome to a tissue (e.g., CNS, heart, liver, and/or muscle). In some embodiments, an AAV particle comprising an AAV capsid variant described herein can be used for delivery of a viral genome to a tissue or cell, e.g., CNS, DRG, heart, liver, or muscle cell or tissue. In some embodiments, an AAV particle of the present disclosure is a recombinant AAV particle. In some embodiments, an AAV particle of the present disclosure is an isolated AAV particle.

The viral genome may encode any payload, such as but not limited to a polypeptide (e.g., a therapeutic polypeptide), an antibody, an enzyme, an RNAi agent and/or components of a gene editing system. In one embodiment, the AAV particles described herein are used to deliver a payload to cells of the CNS, after intravenous delivery. In another embodiment, the AAV particles described herein are used to deliver a payload to cells of the DRG, after intravenous delivery. In some embodiments, the AAV particles described herein are used to deliver a payload to cells of a heart, after intravenous delivery. In some embodiments, the AAV particles described herein are used to deliver a payload to cells of a muscle, e.g., a heart muscle, after intravenous delivery. In some embodiments, the AAV particles described herein are used to deliver a payload to cells of a liver after intravenous delivery.

In some embodiments, a viral genome of an AAV particle comprising an AAV capsid variant, as described herein, comprises a nucleotide sequence comprising a transgene encoding a payload. In some embodiments, the viral genome comprises an inverted terminal repeat sequence (ITR). In some embodiments, the viral genome comprises two ITR sequences, one at the 5′ end of the viral genome (e.g., 5′ relative to the encoded payload) and one at the 3′ end of the viral genome (e.g., 3′ relative to the encoded payload). In some embodiments, a viral genome of an AAV particle, e.g., an AAV particle comprising an AAV capsid variant described herein, may comprise a regulatory element (e.g., promoter), untranslated regions (UTR), a miR binding site, a polyadenylation sequence (polyA), a filler or stuffer sequence, an intron, and/or a linker sequence, e.g., for enhancing transgene expression.

In some embodiments, the viral genome components are selected and/or engineered for expression of the payload in a target tissue (e.g., CNS (e.g., brain, spinal cord, or both), heart, liver, and/or muscle tissue).

### Viral Genome Component: Inverted Terminal Repeats (ITRs)

In some embodiments, the AAV particle comprising an AAV capsid variant described herein comprises a viral genome comprising an ITR and a transgene encoding a payload. In some embodiments, the viral genome comprises two ITRs. In some embodiments, the two ITRs flank the nucleotide sequence encoding the payload at the 5′ and 3′ ends. In some embodiments, the ITRs function as origins of replication comprising recognition sites for replication. In some embodiments, the ITRs comprise sequence regions which can be complementary and symmetrically arranged. In some embodiments, the ITRs incorporated into viral genomes as described herein may be comprised of naturally occurring polynucleotide sequences or recombinantly derived polynucleotide sequences.

In some embodiments, the ITR may be from the same serotype as the capsid polypeptide, e.g., capsid variant, selected from any of the known serotypes, or a variant thereof. In some embodiments, the ITR may be of a different serotype than the capsid. In some embodiments, the viral genome comprises two ITR sequence regions, wherein the ITRs are of the same serotype as one another. In some embodiments, the viral genome comprises two ITR sequence regions, wherein the ITRs are of different serotypes. Non-limiting examples include zero, one, or both of the ITRs having the same serotype as the capsid. In some embodiments, both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 105, 130, 140, 141, 142, 145 nucleotides in length.

### Viral Genome Component: Promoters

In some embodiments, viral genome of an AAV particle described herein comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid variant described herein comprises a viral genome comprising a nucleic acid comprising a transgene encoding a payload, wherein the transgene is operably linked to a promoter. In some embodiments, the promoter is a species-specific promoter, an inducible promoter, a tissue-specific promoter, or a cell cycle-specific promoter (e.g., a promoter as described in Parr et al., Nat. Med.3:1145-9 (1997); the contents of which are herein incorporated by reference in their entirety).

In some embodiments, the promoter may be naturally occurring or non-naturally occurring. Non-limiting examples of promoters include those derived from viruses, plants, mammals, or humans. In some embodiments, the promoters may be those derived from human cells or systems. In some embodiments, the promoter may be truncated or mutated, e.g., a promoter variant.

In some embodiments, the promoter is a ubiquitous promoter, e.g., capable of expression in multiple tissues. In some embodiments the promoter is a human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter. In some embodiments, the promoter is a cell or tissue specific promoter, e.g., capable of expression in tissues or cells of the central or peripheral nervous systems, targeted regions within (e.g., frontal cortex), and/or sub-sets of cells therein (e.g., excitatory neurons). In some embodiments, the promoter is a cell-type specific promoters capable of expression of a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, glial cells, astrocytes, oligodendrocytes, and/or Schwann cells.

In some embodiments, the promoter is a liver specific promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., aMHC, cTnT, and CMV-MLC2k).

In some embodiments, the promoter is a tissue-specific promoter for payload expression in a tissue or cell of the central nervous system. In some embodiments, the promoter is a synapsin (Syn) promoter, glutamate vesicular transporter (VGLUT) promoter, vesicular GABA transporter (VGAT) promoter, parvalbumin (PV) promoter, sodium channel Nav 1.8 promoter, tyrosine hydroxylase (TH) promoter, choline acetyltransferase (ChaT) promoter, methyl-CpG binding protein 2 (MeCP2) promoter, Ca2+/calmodulin-dependent protein kinase II (CaMKII) promoter, metabotropic glutamate receptor 2 (mGluR2) promoter, neurofilament light chain (NFL) or heavy chain (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter, or a fragment thereof. In some embodiments, the promoter is a cell-type specific promoter capable of expression in an astrocyte, e.g., a glial fibrillary acidic protein (GFAP) promoter and a EAAT2 promoter, or a fragment thereof. In some embodiments, the promoter is a cell-type specific promoter capable of expression in an oligodendrocyte, e.g., a myelin basic protein (MBP) promoter or a fragment thereof.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In some embodiments, the promoter of the viral genome described herein (e.g., comprised within an AAV particle comprising an AAV capsid variant described herein) comprises an EF-1a promoter or variant thereof, e.g., as provided in Table 8. In some embodiments, the EF-1a promoter comprises the nucleotide sequence of any one of SEQ ID NOs: 2100, 2101, 2103, 2104, 2108, 2109, or 2111-2120, or a nucleotide sequence provided in Table 8, a nucleotide sequence comprising at least one, two, three, four, five, six or seven but no more than ten modifications, e.g., substitutions, insertions, or deletions, relative to the nucleotide sequence of SEQ ID NOs: 2100, 2101, 2103, 2104, 2108, 2109, or 2111-2120, or a nucleotide sequence provided in Table 8, or a nucleotide sequence with at least 70% (e.g., 80, 85%, 90%, 95%, 96%, 97%, 98%, or 99%) sequence identity to any one of SEQ ID NOs: 2100, 2101, 2103, 2104, 2108, 2109, or 2111-2120, or a nucleotide sequence provided in Table 8.

TABLE 8
Exemplary Promoter Variants

| | | SEQ ID |
|---|---|---|
| Description | Sequences | NO: |
| EF1a Promoter | CGTGAGGCTCCGGTGCCCGTCAGTGGGCAGAGCGCACATCGCCCACAGTCCCCGA | 2100 |
| (intron | GAAGTTGGGGGGAGGGGTCGGCAATTGAACCGGTGCCTAGAGAAGGTGGCGCGGG | |
| underlined) | GTAAACTGGGAAAGTGATGTCGTGTACTGGCTCCGCCTTTTTCCCGAGGGTGGGG | |
| | GAGAACCGTATATAAGTGCAGTAGTCGCCGTGAACGTTCTTTTTCGCAACGGGTT | |
| | TGCCGCCAGAACACAGGTAAGTGCCGTGTGTGGTTCCCGCGGGCCTGGCCTCTTT | |
| | ACGGGTTATGGCCCTTGCGTGCCTTGAATTACTTCCACCTGGCTGCAGTACGTGA | |
| | TTCTTGATCCCGAGCTTCGGGTTGGAAGTGGGTGGGAGAGTTCGAGGCCTTGCGC | |
| | TTAAGGAGCCCCTTCGCCTCGTGCTTGAGTTGAGGCCTGGCCTGGGCGCTGGGGC | |
| | CGCCGCGTGCGAATCTGGTGGCACCTTCGCGCCTGTCTCGCTGCTTTCGATAAGT | |
| | CTCTAGCCATTTAAAATTTTTGATGACCTGCTGCGACGCTTTTTTTCTGGCAAGA | |
| | TAGTCTTGTAAATGCGGGCCAAGATCTGCACACTGGTATTTCGGTTTTTGGGGCC | |
| | GCGGGCGGCGACGGGGCCCGTGCGTCCCAGCGCACATGTTCGGCGAGGCGGGGCC | |
| | TGCGAGCGCGGCCACCGAGAATCGGACGGGGGTAGTCTCAAGCTGGCCGGCCTGC | |
| | TCTGGTGCCTGGCCTCGCGCCGCCGTGTATCGCCCCGCCCTGGGCGGCAAGGCTG | |
| | GCCCGGTCGGCACCAGTTGCGTGAGCGGAAAGATGGCCGCTTCCCGGCCCTGCTG | |
| | CAGGGAGCTCAAAATGGAGGACGCGGCGCTCGGGAGAGCGGGCGGGTGAGTCACC | |
| | CACACAAAGGAAAAGGGCCTTTCCGTCCTCAGCCGTCGCTTCATGTGACTCCACG | |
| | GAGTACCGGGCGCCGTCCAGGCACCTCGATTAGTTCTCGAGCTTTTGGAGTACGT | |
| | CGTCTTTAGGTTGGGGGGAGGGGTTTTATGCGATGGAGTTTCCCCACACTGAGTG | |
| | GGTGGAGACTGAAGTTAGGCCAGCTTGGCACTTGATGTAATTCTCCTTGGAATTT | |
| | GCCCTTTTTGAGTTTGGATCTTGGTTCATTCTCAAGCCTCAGACAGTGGTTCAAA | |
| | GTTTTTTTCTTCCATTTCAGGTGTCGTGA | |
| miniEF1a | GCCCGTCAGTGGGCAGAGCGCACATCGCCCACAGTCCCCGAGAAGTTGGGGGGAG | 2101 |
| | GGGTCGGCAATTGAACCGGTGCCTAGAGAAGGTGGCGCGGGGTAAACTGGGAAAG | |
| | TGATGTCGTGTACTGGCTCCGCCTTTTTCCCGAGGGTGGGGGAGAACCGTATATA | |
| | AGTGCAGTAGTCGCCGTGAACGTTCTTTTTCGCAACGGGTTTGCCGCCAGAACAC | |
| | GCGTAAG | |
| Promoter | GCATG | |
| Variant 1 | | |
| Promoter | GGTGGAGAAGAGCATG | 2103 |
| Variant 2 | | |
| Promoter | GTCATCACTGAGGTGGAGAAGAGCATG | 2104 |
| Variant 3 | | |
| Promoter | CGTGAG | |
| Variant 4 | | |
| Promoter | GT | |
| Variant 5 | | |
| Promoter | GCTCCGGT | |
| Variant 6 | | |
| Promoter | GCCCGTCAGTGGGCAGAGCGCACATCGCCCACAGTCCCCGAGAAGTTGGGGGGAG | 2108 |
| Variant | GGGTCGGCAATTGAACCGGTGCCTAGAGAAGGTGGCGCGGGGTAAACTGGGAAAG | |
| 19 | TGATGTCGTGTACTGGCTCCGCCTTTTTCCCGAGGGTGGGGGAGAACCGTATATA | |
| | AGTGCAGTAGTCGCCGTGAACGTTCTTTTTCGCAACGGGTTTGCCGCCAGAACAC | |
| | AG | |
| Promoter | GCCCGTCAGTGGGCAGAGCGCACATCGCCCACAGTCCCCGAGAAGTTGGGGGGAG | 2109 |
| Variant | GGGTCGGCAATTGAACCGGTGCCTAGAGAAGGTGGCGCGGGGTAAACTGGGAAAG | |
| 20 | TGATGTCGTGTACTGGCTCCGCCTTTTTCCCGAGGGTGGGGGAGAACCGTATATA | |
| | AGTGCAGTAGTCGCCGTGAACGTTCTTTTTCGCAACGGGTTTGCCGCCAGAACAC | |
| | GC | |
| Promoter | GTAAG | |
| Variant 7 | | |
| Promoter | GTGCCCGTCAGTGGGCAGAGCGCACATCGCCCACAGTCCCCGAGAAGTTGGGGGG | 2111 |
| Variant 8 | AGGGGTCGGCAATTGAACCGGTGCCTAGAGAAGGTGGCGCGGGGTAAACTGGGAA | |
| | AGTGATGTCGTGTACTGGCTCCGCCTTTTTCCCGAGGGTGGGGGAGAACCGTATA | |
| | TAAGTGCAGTAGTCGCCGTGAACGTTCTTTTTCGCAACGGGTTTGCCGCCAGAAC | |
| | ACGCGTAAG | |
| Promoter | GCTCCGGTGCCCGTCAGTGGGCAGAGCGCACATCGCCCACAGTCCCCGAGAAGTT | 2112 |
| Variant | GGGGGGAGGGGTCGGCAATTGAACCGGTGCCTAGAGAAGGTGGCGCGGGGTAAAC | |
| 9 | TGGGAAAGTGATGTCGTGTACTGGCTCCGCCTTTTTCCCGAGGGTGGGGGAGAAC | |
| | CGTATATAAGTGCAGTAGTCGCCGTGAACGTTCTTTTTCGCAACGGGTTTGCCGC | |
| | CAGAACACGCGTAAG | |
| Promoter | CGTGAGGCTCCGGTGCCCGTCAGTGGGCAGAGCGCACATCGCCCACAGTCCCCGA | 2113 |
| Variant | GAAGTTGGGGGGAGGGGTCGGCAATTGAACCGGTGCCTAGAGAAGGTGGCGCGGG | |
| 10 | GTAAACTGGGAAAGTGATGTCGTGTACTGGCTCCGCCTTTTTCCCGAGGGTGGGG | |
| | GAGAACCGTATATAAGTGCAGTAGTCGCCGTGAACGTTCTTTTTCGCAACGGGTT | |
| | TGCCGCCAGAACACGCGTAAG | |
| Promoter | CGTGAGGCTCCGGTGCCCGTCAGTGGGCAGAGCGCACATCGCCCACAGTCCCCGA | 2114 |
| Variant | GAAGTTGGGGGGAGGGGTCGGCAATTGAACCGGTGCCTAGAGAAGGTGGCGCGGG | |
| 11 | GTAAACTGGGAAAGTGATGTCGTGTACTGGCTCCGCCTTTTTCCCGAGGGTGGGG | |
| | GAGAACCGTATATAAGTGCAGTAGTCGCCGTGAACGTTCTTTTTCGCAACGGGTT | |
| | TGCCGCCAGAACACAG | |
| Promoter | GCATGCGTGAGGCTCCGGTGCCCGTCAGTGGGCAGAGCGCACATCGCCCACAGTC | 2115 |
| Variant | CCCGAGAAGTTGGGGGGAGGGGTCGGCAATTGAACCGGTGCCTAGAGAAGGTGGC | |
| 12 | GCGGGGTAAACTGGGAAAGTGATGTCGTGTACTGGCTCCGCCTTTTTCCCGAGGG | |
| | TGGGGGAGAACCGTATATAAGTGCAGTAGTCGCCGTGAACGTTCTTTTTCGCAAC | |
| | GGGTTTGCCGCCAGAACACGCGTAAG | |
| Promoter | GCATGCGTGAGGCTCCGGTGCCCGTCAGTGGGCAGAGCGCACATCGCCCACAGTC | 2116 |
| Variant | CCCGAGAAGTTGGGGGGAGGGGTCGGCAATTGAACCGGTGCCTAGAGAAGGTGGC | |
| 13 | GCGGGGTAAACTGGGAAAGTGATGTCGTGTACTGGCTCCGCCTTTTTCCCGAGGG | |
| | TGGGGGAGAACCGTATATAAGTGCAGTAGTCGCCGTGAACGTTCTTTTTCGCAAC | |
| | GGGTTTGCCGCCAGAACACAG | |
| Promoter | GGTGGAGAAGAGCATGCGTGAGGCTCCGGTGCCCGTCAGTGGGCAGAGCGCACAT | 2117 |
| Variant | CGCCCACAGTCCCCGAGAAGTTGGGGGGAGGGGTCGGCAATTGAACCGGTGCCTA | |
| 14 | GAGAAGGTGGCGCGGGGTAAACTGGGAAAGTGATGTCGTGTACTGGCTCCGCCTT | |
| | TTTCCCGAGGGTGGGGGAGAACCGTATATAAGTGCAGTAGTCGCCGTGAACGTTC | |
| | TTTTTCGCAACGGGTTTGCCGCCAGAACACGCGTAAG | |
| Promoter | GGTGGAGAAGAGCATGCGTGAGGCTCCGGTGCCCGTCAGTGGGCAGAGCGCACAT | 2118 |
| Variant 15 | CGCCCACAGTCCCCGAGAAGTTGGGGGGAGGGGTCGGCAATTGAACCGGTGCCTA | |
| | GAGAAGGTGGCGCGGGGTAAACTGGGAAAGTGATGTCGTGTACTGGCTCCGCCTT | |
| | TTTCCCGAGGGTGGGGGAGAACCGTATATAAGTGCAGTAGTCGCCGTGAACGTTC | |
| | TTTTTCGCAACGGGTTTGCCGCCAGAACACAG | |
| Promoter | GTCATCACTGAGGTGGAGAAGAGCATGCGTGAGGCTCCGGTGCCCGTCAGTGGGC | 2119 |
| Variant 16 | AGAGCGCACATCGCCCACAGTCCCCGAGAAGTTGGGGGGAGGGGTCGGCAATTGA | |
| | ACCGGTGCCTAGAGAAGGTGGCGCGGGGTAAACTGGGAAAGTGATGTCGTGTACT | |
| | GGCTCCGCCTTTTTCCCGAGGGTGGGGGAGAACCGTATATAAGTGCAGTAGTCGC | |
| | CGTGAACGTTCTTTTTCGCAACGGGTTTGCCGCCAGAACACGCGTAAG | |
| Promoter | GTCATCACTGAGGTGGAGAAGAGCATGCGTGAGGCTCCGGTGCCCGTCAGTGGGC | 2120 |
| Variant 18 | AGAGCGCACATCGCCCACAGTCCCCGAGAAGTTGGGGGGAGGGGTCGGCAATTGA | |
| | ACCGGTGCCTAGAGAAGGTGGCGCGGGGTAAACTGGGAAAGTGATGTCGTGTACT | |
| | GGCTCCGCCTTTTTCCCGAGGGTGGGGGAGAACCGTATATAAGTGCAGTAGTCGC | |
| | CGTGAACGTTCTTTTTCGCAACGGGTTTGCCGCCAGAACACAG | |

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR (A/G) CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping

UUAUUUA (U/A) (U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo (dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In one embodiment, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprises at least one artificial UTR which is not a variant of a wild type UTR.

In one embodiment, the viral genome of the AAV particle comprises UTRs which have been selected from a family of transcripts whose proteins share a common function, structure, feature or property.

### Viral Genome Component: Polyadenylation Sequence

The viral genome of the AAV particle described herein (e.g., an AAV particle comprising an AAV capsid variant, described herein) may comprise a polyadenylation sequence. In some embodiments, the viral genome of the AAV particle (e.g., an AAV particle comprising an AAV capsid variant, described herein) comprises a polyadenylation sequence between the 3′ end of the nucleotide sequence encoding the payload and the 5′ end of the 3′ITR.

### Viral Genome Component: Introns

In some embodiments, the viral genome of the AAV particle as described herein (e.g., an AAV particle comprising an AAV capsid variant), comprises an element to enhance the payload target specificity and expression (See e.g., Powell et al. Viral Expression Cassette Elements to Enhance Transgene Target Specificity and Expression in Gene Therapy, Discov. Med, 2015, 19 (102): 49-57; the contents of which are herein incorporated by reference in their entirety), such as an intron.

Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

Viral Genome Component: Stuffer sequences

In some embodiments, the viral genome of an AAV particle described herein comprises an element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In some embodiments, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides.

Viral Genome Component: miRNA

In some embodiments, the viral genome comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and β-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles disclosed herein can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome disclosed herein, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleotide sequence encoding a payload), e.g., before the polyA sequence, 5′-UTR region of the viral genome (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, or deletions, but no more than four modifications, e.g., substitutions, insertions, or deletions relative to the nucleotide sequence of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions, insertions, but no more than four modifications, e.g., substitutions, insertions, or deletions relative to the nucleotide sequence of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complementary (e.g., at least 70%, 75%, 80%, 85%, 90%, 95%, 99% or 100% complementary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complementary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183, or a miR1).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 4673), or a nucleotide sequence having at least 50%, 55%, 60%, 65%, 70%, 75%, 80%, 85%, 90%, at least 95%, at least 99%, or 100% sequence identity, or comprising at least one, two, three, four, five, six, or seven modifications, e.g., substitutions, insertions, or deletions, but no more than ten modifications, e.g., insertions, deletions, or substitutions, relative to the nucleotide sequence of SEQ ID NO: 4673, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR 122 binding site series, optionally wherein the encoded miR122 binding site series comprises the nucleotide sequence of:

ACAAACACCATTGTCACACTCCACACAAACACCATTGTCACACTCCACACAAACACCATTGTCACACT CCA (SEQ ID NO: 4674), or a nucleotide sequence having at least 50%, 55%, 60%, 65%, 70%, 75%, 80%, 85%, 90%, at least 95%, at least 99%, or 100% sequence identity, or comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4674, e.g., wherein the modification can result in a mismatch between the encoded miR binding site and the corresponding miRNA. In some embodiments, at least two of the encoded miR 122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions relative to the nucleotide sequence of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4679), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4679, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA.

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and β-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 4675), a nucleotide sequence having at least 50%, 55%, 60%, 65%, 70%, 75%, 80%, 85%, 90%, at least 95%, at least 99%, or 100% sequence identity, or comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4675, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGIGCCATA (SEQ ID NO: 4676), or a nucleotide sequence having at least 50%, 55%, 60%, 65%, 70%, 75%, 80%, 85%, 90%, at least 95%, at least 99%, or 100% sequence identity, or comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4676, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g., an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 4677), a sequence having at least 50%, 55%, 60%, 65%, 70%, 75%, 80%, 85%, 90%, at least 95%, at least 99%, or 100% sequence identity, or comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4677, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA. In some embodiments, the spacer sequence comprises one or more of (i) GGAT; (ii) CACGTG; (iii) GCATGC, or a repeat of one or more of (i)-(iii).

In certain embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 4678), a sequence having at least 50%, 55%, 60%, 65%, 70%, 75%, 80%, 85%, 90%, at least 95%, at least 99%, or 100% sequence identity, or comprising at least one, two, three, four, five, six, or seven modifications, e.g., substitutions, insertions, or deletions, but no more than ten modifications, e.g., substitutions, insertions, or deletions, relative to the nucleotide sequence of SEQ ID NO: 4678, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii)

GCATGC, or a repeat of one or more of (i)-(iii). In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA.

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence comprising at least one, two, or three modifications, e.g., substitutions, insertions, or deletions, but no more than four modifications, e.g., substitutions, insertions, or deletions, relative to the nucleotide sequence of GATAGTTA.

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence comprising one, two, or three modifications, e.g., substitutions, insertions, or deletions, but no more than four modifications, e.g., substitutions, insertions, or deletions, relative to the nucleotide sequence of GATAGTTA.

### Genome Size

In one embodiment, the AAV particle described herein (e.g., an AAV particle comprising an AAV capsid variant), may comprise a single-stranded or double-stranded viral genome. The size of the viral genome may be small, medium, large or the maximum size. As described above, the viral genome may comprise a promoter and a polyA tail.

In one embodiment, the viral genome may be a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In one embodiment, the viral genome may be a small double stranded viral genome. A small double stranded viral genome may be 1.3 to 1.7 kb in size such as, but not limited to, about 1.3, 1.4, 1.5, 1.6, and 1.7 kb in size.

In one embodiment, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In one embodiment, the viral genome may be a medium double stranded viral genome. A medium double stranded viral genome may be 1.8 to 2.1 kb in size such as, but not limited to, about 1.8, 1.9, 2.0, and 2.1 kb in size.

In one embodiment, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In one embodiment, the viral genome may be a large double stranded viral genome. A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Payloads

In some embodiments, an AAV particle of the present disclosure (e.g., an AAV particle comprising an AAV capsid variant described herein) comprises a viral genome comprising a nucleic acid encoding a payload. In some embodiments, the encoded payload is an RNAi agent or a polypeptide. A payload of the present disclosure may be, but is not limited to, a peptide, a polypeptide, a protein, an antibody, an RNAi agent, etc.

In some embodiments, the nucleotide sequence encoding a payload may comprise a combination of coding and non-coding nucleic acid sequences. In some embodiments, the nucleotide sequence encoding the payload may encode a coding or non-coding RNA.

In some embodiments, the AAV particles described herein, e.g., an AAV particle comprising an AAV capsid variant, comprises a nucleic acid encoding a payload. In some embodiments, the encoded payload comprises a therapeutic protein, an antibody, an enzyme, one or more components of a genome editing system, and/or an RNAi agent (e.g., a dsRNA, siRNA, shRNA, pre-miRNA, pri-miRNA, miRNA, stRNA, lncRNA, piRNA, or snoRNA). In some embodiments, the encoded payload modulates, e.g., increases or decreases, the presence, level, and/or activity of a gene, mRNA, protein, or a combination thereof, e.g., in a cell or a tissue.

### Polypeptides

In some embodiments, the encoded payload of the AAV particle comprising an AAV capsid polypeptide, e.g., an AAV capsid variant, described herein comprises a polypeptide, protein, or peptide, e.g., a polypeptide, protein, or peptide described herein. The nucleic acid encoding the payload, may encode a product of any known gene and/or a recombinant version thereof. In some embodiments, the nucleic acid encoding the payload may encode at least one allele of apolipoprotein E (APOE) such as, but not limited to ApoE2, ApoE3 and/or ApoE4. In one embodiment, the nucleic acid encoding the payload encodes ApoE2 (cys112, cys158) protein or a fragment or variant thereof. In one embodiment, the nucleic acid encoding the payload encodes an ApoE3 (cys112, arg158) protein or fragment or variant thereof. In one embodiment, the nucleic acid encoding the payload encodes ApoE4 (arg112, arg158). As another non-limiting example, the encoded payload comprises an aromatic L-amin acid decarboxylase (AADC) protein. As another non-limiting example, the encoded payload comprises an antibody, or a fragment thereof. As another non-limiting example, the encoded payload comprises a human survival of motor neuron (SMN) 1 or SMN2 protein, or fragments or variants thereof. As another non-limiting example, the encoded payload region comprises a glucocerebrosidase (GBA1) protein, or a fragment or variant thereof. As another non-limiting example, the encoded payload comprises a granulin precursor or progranulin (GRN) protein, or a fragment or variant thereof. As another non-limiting example, the encoded payload comprises an aspartoacylase (ASPA) protein, or a fragment or variant thereof. As another non-limiting example, the encoded payload comprises a tripeptidyl peptidase I (CLN2) protein, or a fragment or variant thereof. As another non-limiting example, the encoded payload comprises a beta-galactosidase (GLB1) protein, or a fragment or variant thereof. As another non-limiting example, the encoded payload comprises a N-sulphoglucosamine sulphohydrolase (SGSH) protein, or a fragment or variant thereof. As another non-limiting example, the encoded payload comprises an N-acetyl-alpha-glucosaminidase (NAGLU) protein, or a fragment or variant thereof. As another non-limiting example, the encoded payload comprises an iduronate 2-sulfatase (IDS) protein, or a fragment or variant thereof. As another non-limiting example, the encoded payload comprises an intracellular cholesterol transporter (NPC1) protein, or a fragment or variant thereof. As another non-limiting example, the encoded payload comprises a gigaxonin (GAN) protein, or a fragment or variant thereof. The AAV viral genomes encoding polypeptides described herein may be useful in the fields of human disease, viruses, infections veterinary applications and a variety of in vivo and in vitro settings.

Amino acid sequences of a payload polypeptide encoded by a viral genome described herein, may be translated as a whole polypeptide, a plurality of polypeptides or fragments of polypeptides, which independently may be encoded by one or more nucleic acids, fragments of nucleic acids or variants of any of the aforementioned.

### Antibodies and Antibody Binding Fragments

In some embodiments, the encoded payload of AAV particle comprising an AAV capsid variant described herein comprises an antibody or antibody binding fragment. In some embodiments, the antibody may be a full antibody, a fragment, or any functional variant thereof. As non-limiting examples, an antibody may be a native antibody (e.g., with two heavy and two light chains), a heavy chain variable region, a light chain variable region, a heavy chain constant region, a light chain constant region, Fab, Fab′, F(ab′)₂, Fv, or scFv fragments, a diabody, a linear antibody, a single-chain antibody, a multi-specific antibody, an intrabody, one or more heavy chain complementarity determining regions (CDR), one or more light chain CDRs, a bi-specific antibody, a monoclonal antibody, a polyclonal antibody, a humanized antibody, an antibody mimetic, an antibody variant, a miniaturized antibody, a unibody, a maxibody, and/or a chimeric antigen receptor. The encoded antibody or antibody binding fragment may be useful in the treatment of a neurological disease, a neurodegenerative disorder, a muscular disease, a neuromuscular disorder, a neuro-oncological disorder, or any disorder associated with the central and/or peripheral nervous systems.

In some embodiments, the viral genome of the AAV particle (e.g., an AAV particle comprising an AAV capsid variant described herein) may comprise a nucleic acid which has been engineered to enable or enhance the expression of an antibody, or antibody binding fragment thereof.

In some embodiments, the encoded antibody of the payload of an AAV particle comprising an AAV capsid variant, described herein comprises at least one immunoglobulin variable domain sequence. An antibody may include, for example, full-length, mature antibodies and antigen-binding fragments of an antibody. For example, an antibody can include a heavy (H) chain variable domain sequence (VH), and a light (L) chain variable domain sequence (VL). In another example, an antibody includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments, e.g., an antibody binding fragments, retain the ability to selectively bind with their respective antigen or receptor.

In some embodiments, the antibody binding fragment comprises at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antigen binding fragments include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005).

In some embodiments, the encoded antibody of the payload of an AAV particle described herein comprises a multispecific antibody, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody comprises a third, fourth or fifth immunoglobulin variable domain. In some embodiments, a multispecific antibody is a bispecific antibody, a trispecific antibody, or tetraspecific antibody.

In some embodiments, an encoded multispecific antibody of the payload of an AAV particle described herein is an encoded bispecific antibody. A bispecific antibody has specificity for no more than two antigens. A bispecific antibody is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein).

An antibody or an antibody binding fragment encoded by a viral genome of an AAV particle described herein, may be, but is not limited to, an antibody or antibody fragment that binds to β-amyloid, APOE, tau, SOD1, TDP-43, huntingtin, and/or synuclein. In some embodiments, the encoded payload comprises an antibody or antibody fragment that binds to a neuro-oncology related target, e.g., HER2, EGFR (e.g., EGFRvIII). In some embodiments, the encoded payload comprises an antibody that binds to HER2/neu. In some embodiments, the encoded payload comprises an antibody that binds to β-amyloid. In some embodiments, the encoded payload comprises an antibody that binds to tau.

### Gene Editing System

In some embodiments, the encoded payload of AAV particle comprising an AAV capsid variant described herein comprises a gene editing system or one or more components thereof. In some embodiments, the gene editing system comprises nucleic acid sequences that encode proteins having enzymatic activity to (i) selectively induce double or single stranded breaks in a DNA or RNA sequence, or (ii) substitute, insert or delete a particular base or set of bases of a DNA or RNA sequence in the absence of a double or single stranded break in the DNA or RNA. In some embodiments, the gene editing system includes, but is not limited to a CRISPR-Cas system (including different Cas or Cas-related nucleases), a Zinc finger nuclease, a meganuclease, a TALEN or a base editors. In some embodiments, the gene editing system comprises a chromosomal integration of a transgene, e.g., introduced by a parvovirus vector in the absence of an exogenous nuclease or an enzymatic entity.

RNAi agents

In some embodiments, the encoded payload of AAV particle comprising an AAV capsid variant described herein comprises an RNAi agent, e.g., an RNAi agent described herein. In some embodiments, the encoded payload of a viral genome of an AAV particle comprising an AAV capsid variant described herein comprises an RNAi agent, the RNAi, such as but not limited to, a dsRNA, a siRNA, a shRNA, a pre-miRNA, a pri-miRNA, a miRNA, a stRNA, a lncRNA, a piRNA, or a snoRNA. In some embodiments, the encoded payload comprises an RNAi agent for inhibiting expression of a SOD1, MAPT, APOE, HTT, C9ORF72, TDP-43, APP, BACE, SNCA, ATXN1, ATXN3, ATXN7, SCN1A-SCN5A, or SCN8A-SCN11A gene, protein, and/or mRNA. In some embodiments, the RNAi agent encoded by a viral genome described herein inhibits SOD1, MAPT, APOE, HTT, C9ORF72, TDP-43, APP, BACE, SNCA, ATXN1, ATXN3, ATXN7, SCNIA-SCN5A, or SCN8A-SCN11A.

An AAV particle comprising an AAV capsid variant described herein may comprise a viral genome encoding an RNAi agent, which targets the mRNA of a gene to modulate, e.g., interfere with gene expression and/or protein production.

In some embodiments, the RNAi agent may target a gene at the location of a single-nucleotide polymorphism (SNP) or variant within the nucleotide sequence of the gene.

The RNAi agent may be an siRNA duplex, wherein the siRNA duplex contains an antisense strand (guide strand) and a sense strand (passenger strand) hybridized together forming a duplex structure, wherein the antisense strand is complementary to the nucleic acid sequence of the targeted gene, and wherein the sense strand is homologous to the nucleic acid sequence of the targeted gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

Each strand of an siRNA duplex targeting a gene of interest may be about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length.

In one embodiment, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding the target gene, and the region of complementarity is 30 nucleotides or less, and at least 15 nucleotides in length. Generally, the dsRNA is 19 to 25, 19 to 24 or 19 to 21 nucleotides in length. In some embodiments, the dsRNA is from about 15 to about 25 nucleotides in length, and in other embodiments the dsRNA is from about 25 to about 30 nucleotides in length. In some embodiments, the dsRNA is about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

In some embodiments, the encoded RNAi agent is an siRNA.

In some embodiments, the RNAi agent, e.g., an RNAi agent described herein inhibits the expression of the gene, mRNA, and/or protein by at least 10%, at least 20%, at least 25%, at least 30%, at least 35% or at least 40% or more, such as when assayed by a method known in the art. In some embodiments, the RNAi agent inhibits expression of a gene, mRNA, and protein by 50-100%, e.g., by 30%, 40%, 50%, 60%, 70%, 80%, 85%, 90%, 95% and 100%.

In some embodiments, the AAV particle described herein, comprising a viral genome encoding an RNAi agent targeting a gene of interest is administered to a subject in need for treating and/or ameliorating a disease, e.g., a neurological disorder of any disease associated with the central or peripheral nervous systems.

Design of siRNA

An AAV particle described herein (e.g., an AAV particle comprising an AAV capsid variant described herein) may comprise a viral genome encoding a siRNA molecule (e.g., siRNA duplex or encoded dsRNA) that target a gene of interest and suppress target gene expression, mRNA expression, and protein production. In some aspects, the siRNA molecules are designed and used to knock out target gene variants in cells, e.g., transcripts that are identified in neurological disease. In some aspects, the siRNA molecules are designed and used to knock down target gene variants in cells.

Some guidelines for designing siRNAs (for insertion into a viral genome of the AAV particles described herein) have been proposed in the art. These guidelines generally recommend generating a 19-nucleotide duplexed region, symmetric 2-3 nucleotide 3′overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such considerations, together with the specific sequence of a target gene, highly effective siRNA molecules essential for suppressing mammalian target gene expression may be readily designed.

In one embodiment, the sense and/or antisense strand is designed based on the method and rules outlined in European Patent Publication No. EP1752536, the contents of which are herein incorporated by reference in their entirety. As a non-limiting example, the 3′-terminal base of the sequence is adenine, thymine or uracil. As a non-limiting example, the 5′-terminal base of the sequence is guanine or cytosine. As a non-limiting example, the 3′-terminal sequence comprises seven bases rich in one or more bases of adenine, thymine and uracil.

In one embodiment, an siRNA molecule comprises a sense strand and a complementary antisense strand in which both strands are hybridized together to form a duplex structure. The antisense strand has sufficient complementarity to the target mRNA sequence to direct target-specific RNAi, e.g., the siRNA molecule has a sequence sufficient to trigger the destruction of the target mRNA by the RNAi machinery or process.

In some embodiments, the antisense strand and target mRNA sequences have 100% complementarity. The antisense strand may be complementary to any part of the target mRNA sequence. Neither the identity of the sense sequence nor the homology of the antisense sequence need be 100% complementary to the target.

In other embodiments, the antisense strand and target mRNA sequences comprise at least one mismatch. As a non-limiting example, the antisense strand and the target mRNA sequence have at least 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The siRNA molecule may have a length from about 10-50 or more nucleotides, e.g., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In one embodiment, the siRNA molecule has a length from about 19 to 25, 19 to 24 or 19 to 21 nucleotides.

In some embodiments, the siRNA molecule can be a synthetic RNA duplex comprising about 19 nucleotides to about 25 nucleotides, and two overhanging nucleotides at the 3′-end.

The siRNA molecule may comprise an antisense sequence and a sense sequence, or a fragment or variant thereof. As a non-limiting example, the antisense sequence and the sense sequence have at least 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The sense and antisense sequences may be completely complementary across a substantial portion of their length. In other embodiments, the sense sequence and antisense sequence may be at least 70, 80, 90, 95 or 99% complementary across independently at least 50, 60, 70, 80, 85, 90, 95, or 99% of the length of the strands.

In some embodiments, the sense and antisense strands of a siRNA duplex are linked by a short spacer sequence leading to the expression of a stem-loop structure termed short hairpin RNA (shRNA). The hairpin is recognized and cleaved by Dicer, thus generating mature siRNA molecules.

In some embodiments, the siRNA molecules, as well as associated spacer and/or flanking regions once designed, can be encoded by the viral genome of the AAV particles described herein, for delivery to a cell.

### Molecular Scaffold

In some embodiments, the siRNA molecules may be encoded in a modulatory polynucleotide which also comprises a molecular scaffold.

In some embodiments, the modulatory polynucleotide which comprises the payload (e.g., siRNA, miRNA or other RNAi agent described herein) includes a molecular scaffold which comprises a 5′ flanking sequence, a loop region, and/or a 3′ flanking region. In some embodiments a 5′ or 3′ flanking region may be of any length and may a wild type microRNA sequence or a portion thereof, or may be completely artificial. A 3′ flanking sequence may mirror the 5′ flanking sequence in size and origin. Either flanking sequence may be absent. In one embodiment, both the 5′ and 3′ flanking sequences are absent. The 3′ flanking sequence may optionally contain one or more CNNC motifs, where “N” represents any nucleotide. In some embodiments, the loop comprises at least one UGUG motif. In some embodiments, the UGUG motif is located at the 5′ terminus of the loop. In some embodiments the 5′ and 3′ flanking sequences are the same sequence. In some embodiments they differ by 2%, 3%, 4%, 5%, 10%, 20% or more than 30% when aligned to each other.

In some embodiments, modulatory polynucleotide comprises a stem loop structure. In some embodiments, the modulatory polynucleotide comprises in 5′ to 3′ order: a 5′ flanking sequence, a guide strand sequence, a loop region, a passenger strand sequence, and a 3′ flanking sequence. In some embodiments, the modulatory polynucleotide comprises in 5′ to 3′ order: a 5′ flanking sequence, a passenger strand sequence, a loop region, a guide strand sequence, and a 3′ flanking sequence.

In one embodiment, the molecular scaffold comprises a dual-function targeting modulatory polynucleotide. In one embodiment, the molecular scaffold may comprise one or more linkers known in the art. The linkers may separate regions or one molecular scaffold from another. As a non-limiting example, the molecular scaffold may be polycistronic.

In one embodiment, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

### AAV Production

Viral production disclosed herein describes processes and methods for producing AAV particles (with enhanced, improved and/or increased tropism for a target tissue), e.g., an AAV particle comprising an AAV capsid variant that may be used to contact a target cell to deliver a payload.

In some embodiments, disclosed herein is a method of making AAV particle of the present disclosure, e.g., an AAV particle comprising an AAV capsid variant the method comprising: (i) providing a host cell comprising a viral genome described herein and (ii) incubating the host cell under conditions suitable to enclose the viral genome in an AAV capsid variant, e.g., an AAV capsid variant described herein (e.g., an AAV capsid variant listed in Tables 3, 4, or 5), thereby making the AAV particle. In some embodiments, the method comprises prior to step (i), introducing a first nucleic acid comprising the viral genome into a cell. In some embodiments, the host cell comprises a second nucleic acid encoding the AAV capsid variant. In some embodiments, the second nucleic acid is introduced into the host cell prior to, concurrently with, or after the first nucleic acid molecule. In some embodiments, the AAV particle described herein is an isolated AAV particle. In some embodiments, the AAV particle described herein is a recombinant AAV particle.

Any method known in the art may be used for the preparation of AAV particles. In some embodiments, AAV particles are produced in mammalian cells (e.g., HEK293). In another embodiment, AAV particles are produced in insect cells (e.g., Sf9).

Methods of making AAV particles are well known in the art and are described in e.g., U.S. Pat. Nos. U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir., 219:37-44 (1996); Zhao et al., Vir.272:382-93 (2000); the contents of each of which are herein incorporated by reference in their entirety. In some embodiments, the AAV particles are made using the methods described in International Patent Publication WO2015191508, the contents of which are herein incorporated by reference in their entirety.

### Therapeutic Applications

The present disclosure provides a method for treating a disease, disorder and/or condition in a subject, including a human subject, comprising administering to the subject an AAV particle described herein, e.g., an AAV particle comprising an AAV capsid variant (e.g., an AAV capsid variant described herein), or administering to the subject any of the described compositions, including a pharmaceutical composition, described herein.

In some embodiments, the AAV particle of the present disclosure (e.g., an AAV particle comprising an AAV capsid variant) are administered to a subject prophylactically, to prevent on-set of disease. In another embodiment, the AAV particle (e.g., an AAV particle comprising an AAV capsid variant) of the present disclosure are administered to treat (e.g., lessen the effects of) a disease or symptoms thereof. In yet another embodiment, the AAV particle of the present disclosure (e.g., an AAV particle comprising an AAV capsid variant) are administered to cure (eliminate) a disease. In another embodiment, the AAV particle (e.g., an AAV particle comprising an AAV capsid variant) of the present disclosure are administered to prevent or slow progression of disease. In yet another embodiment, the AAV particle (e.g., an AAV particle comprising an AAV capsid variant) of the present disclosure are used to reverse the deleterious effects of a disease. Disease status and/or progression may be determined or monitored by standard methods known in the art.

In some embodiments, the AAV particle of the disclosure (e.g., an AAV particle comprising an AAV capsid variant) is useful for treatment, prophylaxis, palliation or amelioration of a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein (e.g., an AAV particle comprising an AAV capsid variant described herein) for a gene replacement therapy.

In some embodiments, provided herein is method for treating a neurological disorder and/or neurodegenerative disorder in a subject, comprising administering to the subject an effective amount of a pharmaceutical composition described herein or an AAV particle, e.g., a plurality of particles, comprising an AAV capsid variant described herein. In some embodiments, treatment of a neurological disorder and/or neurodegenerative disorder comprises prevention of said neurological disorder and/or neurological disorder.

In some embodiments, the AAV particle (e.g., an AAV particle comprising an AAV capsid variant) of the disclosure is useful for the treatment, prophylaxis, palliation or amelioration of neurological diseases and/or disorders. In some embodiments, the AAV particle of the disclosure (e.g., an AAV particle comprising an AAV capsid variant) is useful for the treatment, prophylaxis, palliation or amelioration of tauopathy.

In some embodiments, the AAV particle of the disclosure (e.g., an AAV particle comprising an AAV capsid variant) is for the treatment, prophylaxis, palliation or amelioration of Alzheimer's Disease. In some embodiments, treatment of Alzheimer's Disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ApoE2 protein, ApoE4 protein, an ApoE3 protein, BDNF protein, CYP46A1 protein, Klotho protein, fractalkine (FKN) protein, neprilysin protein (NEP), CD74 protein, caveolin-1, or a combination or variant thereof. In some embodiments, treatment of Alzheimer's Disease comprises the use of an AAV particle described herein (e.g., an AAV particle comprising an AAV capsid variant described herein) for a reduction in the expression of a tau gene and/or protein, a synuclein gene and/or protein, or a combination or variant thereof. In some embodiments, the payload encoded by an AAV particle comprising a capsid variant described herein comprises an antibody that binds to tau or synuclein, an RNAi agent for inhibiting tau or synuclein, a gene editing system (e.g., a CRISPR-Cas system) for altering tau or synuclein expression, or a combination thereof.

In some embodiments, the AAV particle of the disclosure (e.g., an AAV particle comprising an AAV capsid variant) is for the treatment, prophylaxis, palliation or amelioration of frontal temporal dementia. In some embodiments, treatment of frontal temporal dementia comprises the use of an AAV particle described herein (e.g., an AAV particle comprising an AAV capsid variant described herein) for a gene replacement therapy. In some embodiments, the payload encoded by an AAV particle comprising a capsid variant described herein comprises a progranulin protein or variant thereof.

In some embodiments, the AAV particle of the disclosure (e.g., an AAV particle comprising an AAV capsid variant) is useful for the treatment, prophylaxis, palliation or amelioration of Parkinson's Disease. In some embodiments, treatment of Parkinson's disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an AADC protein, GAD protein, GDNF protein, TH-GCH1 protein, GBA protein, AIMP2-DX2 protein, or a combination or variant thereof. In some embodiments, treatment of Parkinson's disease comprises the use of an AAV particle described herein (e.g., an AAV particle comprising an AAV capsid variant described herein) for a gene knock-down therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an alpha-synuclein gene, mRNA, and/or protein, or variant thereof.

In some embodiments, the AAV particle of the disclosure (e.g., an AAV particle comprising an AAV capsid variant) is useful for the treatment, prophylaxis, palliation or amelioration of an AADC deficiency. In some embodiments, treatment of AADC deficiency comprises the use of an AAV particle described herein (e.g., an AAV particle comprising an AAV capsid variant described herein) for a gene replacement therapy. In some embodiments, the payload encoded by an AAV particle comprising a capsid variant described herein comprises an AADC protein or variant thereof.

In some embodiments, the AAV particle of the disclosure (e.g., an AAV particle comprising an AAV capsid variant) is useful for the treatment, prophylaxis, palliation or amelioration of Amyotrophic lateral sclerosis (ALS). In some embodiments, treatment of ALS comprises the use of an AAV particle described herein (e.g., an AAV particle comprising an AAV capsid variant described herein) for a gene replacement therapy. In some embodiments, the payload encoded by an AAV particle comprising a capsid variant described herein comprises an TDP-43 protein, UPF1 protein, C9 or f72 protein, CCNF protein, HSF1 protein, Factor H protein, NGF protein, ADAR2 protein, GDNF protein, VEGF protein, HGF protein, NRTN protein, AIMP2-DX2 protein, or a combination or variant thereof. In some embodiments, treatment of ALS comprises the use of an AAV particle described herein (e.g., an AAV particle comprising an AAV capsid variant described herein) for a gene knock-down therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of a SOD1 or C9ORF72 gene, mRNA, and/or protein, or a combination or variant thereof.

In some embodiments, the AAV particle of the disclosure (e.g., an AAV particle comprising an AAV capsid variant) is useful for the treatment, prophylaxis, palliation or amelioration of Huntington's Disease. In some embodiments, treatment of ALS comprises the use of an AAV particle described herein (e.g., an AAV particle comprising an AAV capsid variant described herein) for a gene knock-down (e.g., knock-out) therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an HTT gene, mRNA, and/or protein, or a variant thereof.

In some embodiments, the AAV particle of the disclosure (e.g., an AAV particle comprising an AAV capsid variant) is useful for the treatment, prophylaxis, palliation or amelioration of spinal muscular atrophy. In some embodiments, treatment of spinal muscular atrophy comprises the use of an AAV particle described herein (e.g., an AAV particle comprising an AAV capsid variant described herein) for a gene replacement therapy. In some embodiments, the payload encoded by an

AAV particle comprising a capsid variant described herein comprises an SMN1 protein, an SMN2 protein, or a combination or variant thereof.

In some embodiments, the AAV particle of the disclosure (e.g., an AAV particle comprising an AAV capsid variant) is useful for the treatment, prophylaxis, palliation or amelioration of multiple system atrophy. In some embodiments, treatment of multiple system atrophy comprises the use of an AAV particle described herein (e.g., an AAV particle comprising an AAV capsid variant described herein) for a gene replacement therapy.

In some embodiments, the AAV particle of the disclosure (e.g., an AAV particle comprising an AAV capsid variant) is useful for the treatment, prophylaxis, palliation or amelioration of Gaucher disease (GD) (e.g., Type 1 GD, Type 2 GD, or Type 3 GD). In some embodiments, the AAV particle of the disclosure (e.g., an AAV particle comprising an AAV capsid variant) is useful for the treatment, prophylaxis, palliation or amelioration of Parkinson's disease associated with a GBA mutation. In some embodiments, the AAV particle of the disclosure (e.g., an AAV particle comprising an AAV capsid variant) is useful for the treatment, prophylaxis, palliation or amelioration of dementia with Lewy Bodies (DLB).

In some embodiments, the AAV particle of the disclosure (e.g., an AAV particle comprising an AAV capsid variant) is useful for treatment, prophylaxis, palliation or amelioration of a leukodystrophy, e.g., Alexander disease, autosomal dominant leukodystrophy with autonomic diseases (ADLD), adrenoleukodystrophy (ALD), Canavan disease, cerebrotendinous xanthomatosis (CTX), metachromatic leukodystrophy (MLD), Pelizaeus-Merzbacher disease, or Refsum disease. In some embodiments, treatment of MLD comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ARSA protein or variant thereof. In some embodiments, treatment of ALD comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ABCD-1 protein or variant thereof.

In some embodiments, the AAV particle of the disclosure (e.g., an AAV particle comprising an AAV capsid variant) is useful for the treatment, prophylaxis, palliation, or amelioration of megalencephalic leukoencephalopathy (MLC). In some embodiments, treatment of MLC comprises the use of an AAV particle described herein (e.g., an AAV particle comprising an AAV capsid variant described herein) for a gene replacement therapy. In some embodiments, the payload encoded by an AAV particle comprising a capsid variant described herein comprises an MLC1 protein or variant thereof.

In some embodiments, the AAV particle of the disclosure (e.g., an AAV particle comprising an AAV capsid variant) is useful for the treatment, prophylaxis, palliation, or amelioration of Krabbe disease. In some embodiments, treatment of Krabbe disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GALC protein or variant thereof.

In some embodiments, the AAV particle of the disclosure (e.g., an AAV particle comprising an AAV capsid variant) is useful for the treatment, prophylaxis, palliation, or amelioration of Mucopolysaccharidosis, e.g., a Type I (MPS I), Type II (MPS II), Type IIIA (MPS IIIA), Type IIIB (MPS IIIB), or Type IIIC (MPS IIIC). In some embodiments, treatment of Mucopolysaccharidosis comprises the use of an AAV particle described herein (e.g., an AAV particle comprising an AAV capsid variant described herein) for a gene replacement therapy or a gene editing therapy (e.g., enhancement or correction). In some embodiments, the payload encoded or corrected by an AAV particle comprising a capsid variant described herein comprises an IDUA protein, IDS protein, SGSH protein, NAGLU protein, HGSNAT protein, or a combination or variant thereof.

In some embodiments, the AAV particle of the disclosure (e.g., an AAV particle comprising an AAV capsid variant) is useful for the treatment, prophylaxis, palliation, or amelioration of Batten/NCL. In some embodiments, treatment of Batten/NCL comprises the use of an AAV particle described herein (e.g., an AAV particle comprising an AAV capsid variant described herein) for a gene replacement therapy. In some embodiments, the payload encoded by an AAV particle comprising a capsid variant described herein comprises a CLN1 protein, CLN2 protein, CLN3 protein, CLN5 protein, CLN6 protein, CLN7 protein, CLN8 protein, or a combination or variant thereof.

In some embodiments, the AAV particle of the disclosure (e.g., an AAV particle comprising an AAV capsid variant) is useful for the treatment, prophylaxis, palliation or amelioration of Rett Syndrome. In some embodiments, treatment of Rett Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises an MeCP2 protein or variant thereof.

In some embodiments, the AAV particle of the disclosure (e.g., an AAV particle comprising an AAV capsid variant) is useful for the treatment, prophylaxis, palliation, or amelioration of Angelman Syndrome. In some embodiments, treatment of Angelman Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises a UBE3A protein or variant thereof.

In some embodiments, the AAV particle of the disclosure (e.g., an AAV particle comprising an AAV capsid variant) is useful for the treatment, prophylaxis, palliation, or amelioration of Fragile X Syndrome. In some embodiments, treatment of Fragile X Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises a Reelin protein, a DgkK protein, a FMR1 protein, or a combination or variant thereof.

In some embodiments, the AAV particle of the disclosure (e.g., an AAV particle comprising an AAV capsid variant) is useful for the treatment, prophylaxis, palliation, or amelioration of Canavan Disease. In some embodiments, treatment of Canavan Disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ASPA protein or variant thereof.

In some embodiments, the AAV particle of the disclosure (e.g., an AAV particle comprising an AAV capsid variant) is useful for the treatment, prophylaxis, palliation, or amelioration of a Gangliosidosis, e.g., a GM1 Gangliosidosis or a GM2 Gangliosidosis (e.g., Tay Sachs Sandhoff). In some embodiments, treatment of a Gangliosidosis, e.g., a GM1 Gangliosidosis or a GM2 Gangliosidosis (e.g., Tay Sachs Sandhoff), comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GLB1 protein, a HEXA protein, a HEXB protein, a GM2A protein, or a combination or variant thereof.

In some embodiments, the AAV particle of the disclosure (e.g., an AAV particle comprising an AAV capsid variant) is useful for the treatment, prophylaxis, palliation, or amelioration of GM3 Synthase Deficiency. In some embodiments, treatment of GM3 Synthase Deficiency comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ST3GAL5 protein or variant thereof.

In some embodiments, the AAV particle of the disclosure (e.g., an AAV particle comprising an AAV capsid variant) is useful for the treatment, prophylaxis, palliation, or amelioration of a Niemann-Pick disorder, e.g., a Niemann-Pick A or a Niemann-Pick C1 (NPC-1). In some embodiments, treatment of a Niemann-Pick disorder, e.g., a Niemann-Pick A or a Niemann-Pick C1 (NPC-1) comprises the use of an AAV particle described herein (e.g., an AAV particle comprising an AAV capsid variant described herein) for a gene replacement therapy. In some embodiments, the payload encoded by an AAV particle comprising a capsid variant described herein comprises an ASM protein, an NPC1 protein, or variant thereof.

In some embodiments, the AAV particle of the disclosure (e.g., an AAV particle comprising an AAV capsid variant) is useful for the treatment, prophylaxis, palliation, or amelioration of Schwannoma (e.g., Neuroma). In some embodiments, treatment of Schwannoma (e.g., Neuroma) comprises the use of an AAV particle described herein (e.g., an AAV particle comprising an AAV capsid variant described herein) for a gene replacement therapy. In some embodiments, the payload encoded by an AAV particle comprising a capsid variant described herein comprises a Caspase-1 protein or variant thereof.

In some embodiments, the AAV particle of the disclosure (e.g., an AAV particle comprising an AAV capsid variant) is useful for the treatment, prophylaxis, palliation, or amelioration of a Tuberous Sclerosis, e.g., Tuberous Sclerosis Type 1 or Tuberous Sclerosis Type 2. In some embodiments, treatment of Tuberous Sclerosis, e.g., Tuberous Sclerosis Type 1 or Tuberous Sclerosis Type 2 comprises the use of an AAV particle described herein (e.g., an AAV particle comprising an AAV capsid variant described herein) for a gene replacement therapy. In some embodiments, the payload encoded by an AAV particle comprising a capsid variant described herein comprises a TSC1 protein, a TSC2 protein, or variant thereof.

In some embodiments, the AAV particle of the disclosure (e.g., an AAV particle comprising an AAV capsid variant) is useful for the treatment, prophylaxis, palliation, or amelioration of a CDKL5 Deficiency. In some embodiments, treatment of a CDKL5 Deficiency comprises the use of an AAV particle described herein (e.g., an AAV particle comprising an AAV capsid variant described herein) for a gene replacement therapy. In some embodiments, the payload encoded by an AAV particle comprising a capsid variant described herein comprises a CDKL5 protein or variant thereof.

In some embodiments, the AAV particle of the disclosure (e.g., an AAV particle comprising an AAV capsid variant) is useful for the treatment, prophylaxis, palliation, or amelioration of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder. In some embodiments, treatment of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder, comprises the use of an AAV particle described herein (e.g., an AAV particle comprising an AAV capsid variant described herein) for a gene replacement therapy. In some embodiments, the payload encoded by an AAV particle comprising a capsid variant described herein comprises a GJB1 protein, a MFN2 protein, a FIG. 4 protein, or variant thereof.

In some embodiments, the AAV particle of the disclosure (e.g., an AAV particle comprising an AAV capsid variant) is useful for the treatment, prophylaxis, palliation, or amelioration of an Aspartylglucosaminuria (AGU). In some embodiments, treatment of an AGU comprises the use of an AAV particle described herein (e.g., an AAV particle comprising an AAV capsid variant described herein) for a gene replacement therapy. In some embodiments, the payload encoded by an AAV particle comprising a capsid variant described herein comprises an AGA protein or variant thereof.

In some embodiments, the AAV particle of the disclosure (e.g., an AAV particle comprising an AAV capsid variant) is useful for the treatment, prophylaxis, palliation, or amelioration of a Leigh Syndrome. In some embodiments, treatment of a Leigh Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises a SURF1 protein or variant thereof.

In some embodiments, the AAV particle of the disclosure (e.g., an AAV particle comprising an AAV capsid variant) is useful for the treatment, prophylaxis, palliation, or amelioration of epilepsy. In some embodiments, treatment of epilepsy comprises the use of an AAV particle described herein (e.g., an AAV particle comprising an AAV capsid variant described herein) for a gene replacement therapy. In some embodiments, the payload encoded by an AAV particle comprising a capsid variant described herein comprises an NPY/Y2 protein, a Galanin protein, a Dynorphin protein, an AIMP2-DX2 protein, an SLC6A1 protein, an SLC13A5 protein, a KCNQ2 protein, or variant thereof.

In some embodiments, the AAV particle of the disclosure (e.g., an AAV particle comprising an AAV capsid variant) is useful for the treatment, prophylaxis, palliation, or amelioration of a Dravet Syndrome. In some embodiments, treatment of Dravet Syndrome comprises the use of an AAV particle described herein (e.g., an AAV particle comprising an AAV capsid variant described herein) for a gene replacement therapy. In some embodiments, the payload encoded by an AAV particle comprising a capsid variant described herein comprises an SCN1a protein, or variant thereof.

In some embodiments, the AAV particle of the disclosure (e.g., an AAV particle comprising an AAV capsid variant) is useful for the treatment, prophylaxis, palliation, or amelioration of a Duchenne muscular dystrophy (DMD). In some embodiments, treatment of DMD comprises the use of an AAV particle described herein (e.g., an AAV particle comprising an AAV capsid variant described herein) for a gene replacement therapy or enhancement (e.g., correction of exon-skipping), or a gene editing therapy (e.g., enhancement or correction). In some embodiments, the payload encoded or corrected by an AAV particle comprising a capsid variant described herein comprises a Dystrophin gene and/or protein, a Utrophin gene and/or protein, or a GALGT2 gene and/or protein, or a Follistatin gene and/or protein, or a combination or variant thereof.

In some embodiments, the AAV particle of the disclosure (e.g., an AAV particle comprising an AAV capsid variant) is useful for the treatment, prophylaxis, palliation, or amelioration of Pompe Disease. In some embodiments, treatment of Pompe Disease comprises the use of an AAV particle described herein (e.g., an AAV particle comprising an AAV capsid variant described herein) for a gene replacement therapy. In some embodiments, the payload encoded by an AAV particle comprising a capsid variant described herein comprises an alpha-glucosidase (GAA) protein, or variant thereof.

In some embodiments, the AAV particle of the disclosure (e.g., an AAV particle comprising an AAV capsid variant) is useful for the treatment, prophylaxis, palliation, or amelioration of Limb-Girdle Muscular Dystrophy (LGMD2A). In some embodiments, treatment of LGMD2A comprises the use of an AAV particle described herein (e.g., an AAV particle comprising an AAV capsid variant described herein) for a gene replacement therapy. In some embodiments, the payload encoded by an AAV particle comprising a capsid variant described herein comprises a calpain-3 (CAPN-3) protein, a dysferlin (DYSF) protein, a gamma-sarcoglycan (SGCG) protein, an alpha-sarcoglycan (SGCA) protein, a microdystrophin protein, a dystrophin protein, a beta-sarcoglycan (SGCB) protein, a fukutin-related protein (FKRP) protein, an anoctamin-5 (ANO5) protein, or a combination or variant thereof.

In some embodiments, the AAV particle of the disclosure (e.g., an AAV particle comprising an AAV capsid variant) is useful for the treatment, prophylaxis, palliation, or amelioration of a titinopathy. In some embodiments, treatment of titinopathy comprises the use of an AAV particle described herein (e.g., an AAV particle comprising an AAV capsid variant described herein) for a gene replacement therapy. In some embodiments, the payload encoded by an AAV particle comprising a capsid variant described herein comprises a titin protein or variant thereof.

In some embodiments, the AAV particle of the disclosure (e.g., an AAV particle comprising an AAV capsid variant) is useful for the treatment, prophylaxis, palliation, or amelioration of an X-linked myotubular myopathy. In some embodiments, treatment of X-linked myotubular myopathycomprises the use of an AAV particle described herein (e.g., an AAV particle comprising an AAV capsid variant described herein) for a gene replacement therapy. In some embodiments, the payload encoded by an AAV particle comprising a capsid variant described herein comprises a myotubularin protein (e.g., encoded by the MTM1 gene) or variant thereof.

In some embodiments, the AAV particle of the disclosure (e.g., an AAV particle comprising an AAV capsid variant) is useful for the treatment, prophylaxis, palliation, or amelioration of chronic or neuropathic pain.

In some embodiments, the AAV particle of the disclosure (e.g., an AAV particle comprising AAV capsid variant) is useful for treatment, prophylaxis, palliation, or amelioration of a disease associated with the central nervous system.

In some embodiments, the AAV particle of the disclosure (e.g., an AAV particle comprising an AAV capsid variant) is useful for treatment, prophylaxis, palliation, or amelioration of a disease associated with the peripheral nervous system.

In some embodiments, the AAV particle of the disclosure (e.g., an AAV particle comprising an AAV capsid variant) is useful for treatment, prophylaxis, palliation, or amelioration of a neuro-oncological disorder in a subject. In some embodiments, treatment of a neuro-oncological disorder comprises prevention of said neuro-oncological disorder. In some embodiments, a neuro-oncological disorder comprises a cancer of a primary CNS origin (e.g., a CNS cell, a tissue, or a region), or a metastatic cancer in a CNS cell, tissue, or region. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, the AAV particle of the disclosure (e.g., an AAV particle comprising an AAV capsid variant) is useful for the treatment, prophylaxis, palliation, or amelioration of a disease associated with expression of HER2, e.g., a disease associated with overexpression of HER2. In some embodiments, the AAV particle of the disclosure (e.g., an AAV particle comprising an AAV capsid variant) is useful for the treatment, prophylaxis, palliation, or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, the AAV particle of the disclosure (e.g., an AAV particle comprising an AAV capsid variant) is useful for treatment, prophylaxis, palliation, or amelioration of a muscular disorder and/or neuromuscular disorder in a subject. In some embodiments, treatment of a muscular disorder and/or neuromuscular disorder comprises prevention of said muscular disorder and/or neuromuscular disorder.

In some embodiments, the AAV particle of the disclosure (e.g., an AAV particle comprising an AAV capsid variant) is useful for treatment, prophylaxis, palliation or amelioration of a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction. In some embodiments, the cardiac disease is a disease associated with expression, e.g., aberrant expression, of LAMP2B, MYBPC3, TNNI3, LMNA, BAG3, DWORF, PKP2, Cx43, TAZ, CASQ2, SERCA2a, I-1c, S100A1 and/or ARC, S100A1, ASCL1, miR133, Mydelta3, Sav, or a combination or variant thereof. In some embodiments, treatment of a cardiac disorder described herein comprises the use of an AAV particle described herein (e.g., an AAV particle comprising an AAV capsid variant described herein) for a gene replacement therapy.

In some embodiments, the cardiac disease is a genetic disorder, e.g., an autosomal dominant genetic disorder, an autosomal recessive disorder, or an X-linked recessive genetic disorder. In some embodiments, the cardiomyopathy is a genetic disorder, e.g., a genetic disorder associated with an abnormality (e.g., mutation, insertion, rearrangement and/or deletion) in a gene chosen from TTN, LMNA, MYH7, MYH6, SCN5A, TNNT2, RBM20, TNNI3, MYL2, MYL3, PKP2, DSP, DSG2, DSC2, JUP, or a combination thereof. In some embodiments, the cardiac disorder is a dilated cardiomyopathy, e.g., a dilated cardiomyopathy associated with an abnormality (e.g., mutation, insertion, rearrangement and/or deletion) in a gene chosen from TTN, LMNA, MIH7, BAG3, MIPN, TNNT2, SCN5A, RBN20, TNPO, LAMA4, VCL, LDB3, TCAP, PSEN1/2, ACTN2, CRYAB, TPM1, ABCC9, ACTC1, PDLIM3, ILK, TNNC1, TNNI3, PLN, DES, SGCD, CSRP3, MIH6, EYA4, ANKRD1, DMD, GATAD1, TAZ/G4.5, or combination thereof. In some embodiments, the cardiac disorder is a hypertrophic cardiomyopathy, e.g., a hypertrophic cardiomyopathy associated with an abnormality (e.g., mutation, insertion, rearrangement and/or deletion) in a gene chosen from MYH7, TNNT2, TNNI3, TPM1, MYL2, MYL3, ACTC1, CSRP3, TTN, ACTN2, MYH6, TCAP, TNNC1, or a combination thereof. In some embodiments, the cardiac disorder is an arrhythmogenic ventricular cardiomyopathy, e.g., an arrhythmogenic ventricular cardiomyopathy associated with an abnormality (e.g., mutation, insertion, rearrangement and/or deletion) in a gene chosen from PKP2, DSG2, DSP, RYR2, DSC2, TGFB3, TMEM43, DES, TTN, LMNA, or a combination thereof.

In some embodiments, the AAV particle of the present disclosure (e.g., an AAV particle comprising an AAV capsid polypeptide, e.g., an AAV capsid variant) is administered to a subject having at least one of the diseases or symptoms described herein. In some embodiments, an AAV particle of the present disclosure is administered to a subject having or diagnosed with having a disease or disorder described herein.

Any neurological disease or disorder, neurodegenerative disorder, muscular disorder, neuromuscular disorder, and/or neuro-oncological disorder may be treated with the AAV particles of the disclosure, or pharmaceutical compositions thereof.

### Pharmaceutical Composition and Formulations

According to the present disclosure, an AAV particle comprising an AAV capsid variant described herein may be prepared as a pharmaceutical composition. In some embodiments, the pharmaceutical composition comprises at least one active ingredients. In some embodiments, the pharmaceutical composition comprises a pharmaceutically acceptable excipient.

In some embodiments, an AAV particle of the present disclosure (e.g., an AAV particle comprising an AAV capsid polypeptide, e.g., an AAV capsid variant) can be formulated using an excipient to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload. Formulations of the present disclosure can include, without limitation, saline, liposomes, lipid nanoparticles, polymers, peptides, proteins, cells transfected with viral vectors (e.g., for transfer or transplantation into a subject) and combinations thereof.

In some embodiments, the relative amount of the active ingredient (e.g., an AAV particle comprising an AAV capsid variant described herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between .5 and 50%, between 1-30%, between 5-80%, at least 80% (w/w) active ingredient.

In some embodiments, the pharmaceutical composition comprising an AAV particle described herein may comprise an AAV capsid variant and a viral genome encoding a payload, e.g., a payload described herein, with or without a pharmaceutically acceptable excipient.

The present disclosure also provides in some embodiments, a pharmaceutical composition suitable for administration to a subject, e.g., a human. In some embodiments, the pharmaceutical composition is administered to a subject, e.g., a human.

### Administration

In some embodiments, an AAV particle disclosed herein (e.g., an AAV particle comprising an AAV capsid variant) may be administered by a to a subject by a delivery route, e.g., a localized delivery route or a systemic delivery route.

In some embodiments, an AAV particle described herein (e.g., an AAV particle comprising an AAV capsid variant) may be administered via such a route that it is able to cross the blood-brain barrier, vascular barrier, or other epithelial barrier. In some embodiments, an AAV particle of the present disclosure (e.g., an AAV particle comprising an AAV capsid variant) may be administered in any suitable form, either as a liquid solution or suspension, as a solid form suitable for liquid solution or suspension in a liquid solution. In some embodiments, an AAV particle (e.g., an AAV particle comprising an AAV capsid variant) may be formulated with any appropriate and pharmaceutically acceptable excipient.

In some embodiments, the AAV particle described herein (e.g., an AAV particle comprising an AAV capsid variant) is administered intramuscularly, intravenously, intracerebrally, intrathecally, intracerebroventricularly, via intraparenchymal administration, or via intra-cisterna magna injection (ICM). In some embodiments, the AAV particle described herein (e.g., an AAV particle comprising an AAV capsid variant) is administered intramuscularly. In some embodiments, the AAV particle described herein (e.g., an AAV particle comprising an AAV capsid variant) is administered intravenously.

In some embodiments, an AAV particle of the present disclosure (e.g., an AAV particle comprising an AAV capsid variant) may be delivered to a subject via a single route administration. In some embodiments, an AAV particle of the present disclosure may be delivered to a subject via a multi-site route of administration. In some embodiments, a subject may be administered at 2, 3, 4, 5, or more than 5 sites.

In some embodiments, an AAV particle of the present disclosure (e.g., an AAV particle comprising an AAV capsid variant) is administered via a bolus infusion. In some embodiments, an AAV particle of the present disclosure is administered via sustained delivery over a period of minutes, hours, or days. In some embodiments, the infusion rate may be changed depending on the subject, distribution, formulation, and/or another delivery parameter. In some embodiments, an AAV particle of the present disclosure is administered using a controlled release. In some embodiments, an AAV particle of the present disclosure is administered using a sustained release, e.g., a release profile that conforms to a release rate over a specific period of time.

In some embodiments, an AAV particle (e.g., an AAV particle comprising an AAV capsid variant) may be delivered by more than one route of administration. As non-limiting examples of combination administrations, an AAV particle may be delivered by intrathecal and intracerebroventricular, or by intravenous and intraparenchymal administration.

### Intravenous Administration

In some embodiments, an AAV particle described herein (e.g., an AAV particle comprising an AAV capsid polypeptide, e.g., an AAV capsid variant) may be administered to a subject by systemic administration. In some embodiments, the systemic administration is intravenous administration. In another embodiment, the systemic administration is intraarterial administration. In some embodiments, an AAV particle of the present disclosure may be administered to a subject by intravenous administration. In some embodiments, the intravenous administration may be achieved by subcutaneous delivery. In some embodiments, the AAV particle is administered to the subject via focused ultrasound (FUS), e.g., coupled with the intravenous administration of microbubbles (FUS-MB) or MRI-guided FUS coupled with intravenous administration, e.g., as described in Terstappen et al. (Nat Rev Drug Discovery, doi.org/10.1038/s41573-021-00139-y (2021)), the contents of which are incorporated herein by reference in its entirety. In some embodiments, the AAV particle, e.g., an AAV particle described herein, is administered to the subject intravenously. In some embodiments, the subject is a human.

### Administration to the CNS

In some embodiments, an AAV particle described herein (e.g., an AAV particle comprising an AAV capsid variant) may be delivered by direct injection into the brain. As a non-limiting example, the brain delivery may be by intrahippocampal administration. In some embodiments, an AAV particle of the present disclosure may be administered to a subject by intraparenchymal administration. In some embodiments, the intraparenchymal administration is to tissue of the central nervous system. In some embodiments, an AAV particle of the present disclosure may be administered to a subject by intracranial delivery (See, e.g., U.S. Pat. No. 8,119,611; the content of which is incorporated herein by reference in its entirety). In some embodiments, an AAV particle described herein may be delivered by injection into the CSF pathway. Non-limiting examples of delivery to the CSF pathway include intrathecal and intracerebroventricular administration. In some embodiments, an AAV particle described herein may be administered via intracisternal magna (ICM) injection.

In some embodiments, an AAV particle of the present disclosure (e.g., an AAV particle comprising an AAV capsid variant) may be delivered to the brain by systemic delivery. As a non-limiting example, the systemic delivery may be by intravascular administration. As a non-limiting example, the systemic or intravascular administration may be intravenous.

In some embodiments, an AAV particle (e.g., an AAV particle comprising an AAV capsid variant) of the present disclosure may be delivered by an intraocular delivery route. A non-limiting example of an intraocular administration includes an intravitreal injection.

### Intramuscular Administration

In some embodiments, an AAV particle described herein (e.g., an AAV particle comprising an AAV capsid variant) may be delivered by intramuscular administration. Without wishing to be bound by theory, it is believed in some embodiments, that the multi-nucleated nature of muscle cells provides an advantage to gene transduction subsequent to AAV delivery. In some embodiments, cells of the muscle are capable of expressing recombinant proteins with the appropriate post-translational modifications. Without wishing to be bound by theory, it is believed in some embodiments, the enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered. In some embodiments, an AAV particle of the present disclosure may be delivered by an intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, an AAV particle of the present disclosure (e.g., an AAV particle comprising an AAV capsid variant) is administered to a subject and transduces the muscle of a subject. As a non-limiting example, an AAV particle is administered by intramuscular administration. In some embodiments, an AAV particle of the present disclosure may be administered to a subject by subcutaneous administration. In some embodiments, the intramuscular administration is via systemic delivery. In some embodiments, the intramuscular administration is via intravenous delivery. In some embodiments, the intramuscular administration is via direct injection to the muscle.

In some embodiments, the muscle is transduced by administration, e.g., intramuscular administration. In some embodiments, an intramuscular delivery comprises administration at one site. In some embodiments, an intramuscular delivery comprises administration at more than one site. In some embodiments, an intramuscular delivery comprises administration at two, three, four, or more sites. In some embodiments, intramuscular delivery is combined with at least one other method of administration.

In some embodiments, an AAV particle pf the present disclosure (e.g., an AAV particle comprising an AAV capsid variant) may be administered to a subject by peripheral injections. Non-limiting examples of peripheral injections include intraperitoneal, intramuscular, intravenous, conjunctival, or joint injection. It was disclosed in the art that the peripheral administration of AAV particles can be transported to the central nervous system, for example, to the motor neurons (e.g., U.S. Patent Publication Nos. 20100240739 and 20100130594; the content of each of which is incorporated herein by reference in their entirety).

In some embodiments, an AAV particle of the present disclosure (e.g., an AAV particle comprising an AAV capsid variant) may be administered to a subject by intraparenchymal administration. In some embodiments, the intraparenchymal administration is to muscle tissue. In some embodiments, an AAV particle of the present disclosure is delivered as described in Bright et al 2015 (Neurobiol Aging. 36 (2): 693-709), the contents of which are herein incorporated by reference in their entirety. In some embodiments, an AAV particle of the present disclosure is administered to the gastrocnemius muscle of a subject. In some embodiments, an AAV particle of the present disclosure is administered to the bicep femorii of the subject. In some embodiments, an AAV particles of the present disclosure is administered to the tibialis anterior muscles. In some embodiments, an AAV particle of the present disclosure is administered to the soleus muscle.

### Depot Administration

As described herein, in some embodiments, a pharmaceutical composition and/or an AAV particle of the present disclosure (e.g., an AAV particle comprising an AAV capsid polypeptide, e.g., an AAV capsid variant) are formulated in depots for extended release. Generally, specific organs or tissues are targeted for administration.

In some embodiments, a pharmaceutical composition and/or an AAV particle of the present disclosure (e.g., an AAV particle comprising an AAV capsid polypeptide, e.g., an AAV capsid variant) are spatially retained within or proximal to target tissues. Provided are methods of providing a pharmaceutical composition, an AAV particle, to target tissues of mammalian subjects by contacting target tissues (which comprise one or more target cells) with the pharmaceutical composition and/or the AAV particle, under conditions such that they are substantially retained in target tissues, e.g., such that at least 10, 20, 30, 40, 50, 60, 70, 80, 85, 90, 95, 96, 97, 98, 99, 99.9, 99.99 or greater than 99.99% of the composition is retained in the target tissues. In some embodiments, retention is determined by measuring the amount of pharmaceutical composition and/or AAV particle, that enter a target cell or a plurality of target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of a pharmaceutical composition and/or an AAV particle, administered to a subject are present intracellularly at a period of time following administration. For example, intramuscular injection to a subject may be performed using aqueous compositions comprising a pharmaceutical composition and/or an AAV particle of the present disclosure and a transfection reagent, and retention is determined by measuring the amount of the pharmaceutical composition and/or the AAV particle, present in the muscle cell or plurality of muscle cells.

In some embodiments, disclosed herein are methods of providing a pharmaceutical composition and/or an AAV particle of the present disclosure (e.g., an AAV particle comprising an AAV capsid polypeptide, e.g., an AAV capsid variant) to a tissue of a subject, by contacting the tissue (comprising a cell, e.g., a plurality of cells) with the pharmaceutical composition and/or the AAV particle under conditions such that they are substantially retained in the tissue. In some embodiments, a pharmaceutical composition and/or AAV particle described herein comprise a sufficient amount of an active ingredient such that the effect of interest is produced in at least one cell. In some embodiments, a pharmaceutical composition and/or an AAV particle generally comprise one or more cell penetration agents. In some embodiments, the disclosure provides a naked formulations (such as without cell penetration agents or other agents), with or without pharmaceutically acceptable carriers.

### Methods of Treatment

Provided in the present disclosure are methods for introducing (e.g., delivering) an AAV particle of the present disclosure (e.g., an AAV particle comprising an AAV capsid variant described herein) into cells. In some embodiments, the method comprises introducing into said cells an AAV particle or vector described herein in an amount sufficient to modulate, e.g., increase, the production of a target gene, mRNA, and/or protein. In some embodiments, the method comprises introducing into said cells an AAV particle or vector described herein in an amount sufficient to modulate, e.g., decrease, expression of a target gene, mRNA, and/or protein. In some embodiments, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes. In other embodiments, the cells may be a muscle cell (e.g., a cell of a diaphragm, a quadriceps, or a heart (e.g., a heart atrium or a heart ventricle)) or a liver cell. In some embodiments, the cell may be a heart cell (e.g., a cell of a heart atrium or a cell of a heart ventricle).

Disclosed in the present disclosure are methods for treating a neurological disease/disorder or a neurodegenerative disorder, a muscular or neuromuscular disorder, or a neuro-oncological disorder associated with aberrant, e.g., insufficient or increased, function/presence of a protein, e.g., a target protein in a subject in need of treatment.

In some embodiments, the method comprises administering to the subject a therapeutically effective amount of a composition comprising AAV particles of the present disclosure. As a non-limiting example, the AAV particles can increase target gene expression, increase target protein production, and thus reduce one or more symptoms of neurological disease in the subject such that the subject is therapeutically treated.

In other embodiments, the method comprises administering to the subject a therapeutically effective amount of a composition comprising AAV particles (e.g., an AAV particle comprising an AAV capsid polypeptide, e.g., an AAV capsid variant) comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles of the present disclosure (e.g., an AAV particle comprising an AAV capsid variant described herein) is administered to the central nervous system of the subject via systemic administration. In some embodiments, the systemic administration is intravenous (IV) injection. In some embodiments, the AAV particle described herein or a pharmaceutical composition comprising an AAV particle described herein is administered by focused ultrasound (FUS), e.g., coupled with the intravenous administration of microbubbles (FUS-MB) or MRI-guided FUS coupled with intravenous administration.

In some embodiments, the composition comprising the AAV particle of the present disclosure (e.g., an AAV particle comprising an AAV capsid variant) is administered to the central nervous system of the subject via intraventricular administration. In some embodiments, the composition comprising the AAV particle of the present disclosure (e.g., an AAV particle comprising an AAV capsid variant) is administered via intra-cisterna magna injection (ICM).

In some embodiments, the composition comprising an AAV particle of the present disclosure (e.g., an AAV particle comprising an AAV capsid variant) is administered to the central nervous system of the subject via intraventricular injection and intravenous injection.

In some embodiments, the composition comprising the AAV particle of the present disclosure (e.g., an AAV particle comprising an AAV capsid variant) is administered to the central nervous system of the subject via ICM injection and intravenous injection at a specific dose per subject. As a non-limiting example, the AAV particles are administered via ICM injection at a dose of 1×10⁴ VG per subject. As a non-limiting example, the AAV particles are administered via IV injection at a dose of 2×10¹³ VG per subject.

In some embodiments, the composition comprising the AAV particle of the present disclosure (e.g., an AAV particle comprising an AAV capsid variant) is administered to the central nervous system of the subject. In other embodiments, the composition comprising the AAV particles of the present disclosure is administered to a CNS tissue of a subject (e.g., putamen, hippocampus, thalamus, or cortex of the subject).

In some embodiments, the composition comprising the AAV particle of the present disclosure (e.g., an AAV particle comprising an AAV capsid variant) is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In some embodiments, the composition comprising the AAV particle of the present disclosure (e.g., an AAV particle comprising an AAV capsid variant) is administered to the central nervous system of the subject via intraparenchymal injection and intravenous injection.

In some embodiments, the composition comprising the AAV particle of the present disclosure (e.g., an AAV particle comprising an AAV capsid variant) is administered to the central nervous system of the subject via intraventricular injection, intraparenchymal injection and intravenous injection.

In some embodiments, the composition comprising an AAV particle (e.g., an AAV particle comprising an AAV capsid variant) of a plurality of particles of the present disclosure is administered to a muscle of the subject via intravenous injection. In some embodiments, the composition comprising an AAV particle of a plurality of particles of the present disclosure is administered to a muscle of the subject via intramuscular injection.

In some embodiments, an AAV particle of the present disclosure (e.g., an AAV particle comprising an AAV capsid variant) may be delivered into specific types of cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells. In some embodiments, an AAV particle of the present disclosure may be delivered into a muscle cell, e.g., a cell of the quadriceps, diaphragm, liver, and/or heart (e.g., heart atrium or heart ventricle).

In some embodiments, an AAV particle (e.g., an AAV particle comprising an AAV capsid polypeptide, e.g., an AAV capsid variant), e.g., a plurality of particles, of the present disclosure may be delivered to a cell or region of the midbrain. In some embodiments, an AAV particle (e.g., an AAV particle comprising an AAV capsid polypeptide, e.g., an AAV capsid variant), e.g., a plurality of particles, of the present disclosure may be delivered to a cell or region of the brains stem.

In some embodiments, an AAV particle (e.g., an AAV particle comprising an AAV capsid polypeptide, e.g., an AAV capsid variant), e.g., a plurality of particles, of the present disclosure may be delivered to neurons in the putamen, hippocampus, thalamus and/or cortex.

In some embodiments, an AAV particle (e.g., an AAV particle comprising an AAV capsid variant), e.g., a plurality of particles, of the present disclosure may be used as a therapy for a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein (e.g., an AAV particle comprising an AAV capsid variant described herein) for a gene replacement therapy.

In some embodiments, an AAV particle (e.g., an AAV particle comprising an AAV capsid variant), e.g., a plurality of particles, of the present disclosure may be used as a therapy for a neurological disease.

In some embodiments, an AAV particle (e.g., an AAV particle comprising an AAV capsid variant), e.g., a plurality of particles, of the present disclosure may be used as a therapy for tauopathies.

In some embodiments, an AAV particle (e.g., an AAV particle comprising an AAV capsid variant), e.g., a plurality of particles, of the present disclosure may be used as a therapy for Alzheimer's Disease.

In some embodiments, an AAV particle (e.g., an AAV particle comprising an AAV capsid variant), e.g., a plurality of particles, of the present disclosure may be used as a therapy for Amyotrophic Lateral Sclerosis.

In some embodiments, an AAV particle (e.g., an AAV particle comprising an AAV capsid variant), e.g., a plurality of particles, of the present disclosure may be used as a therapy for Huntington's Disease.

In some embodiments, an AAV particle (e.g., an AAV particle comprising an AAV capsid variant), e.g., a plurality of particles, of the present disclosure may be used as a therapy for Parkinson's Disease.

In some embodiments, an AAV particle (e.g., an AAV particle comprising an AAV capsid variant), e.g., a plurality of particles, of the present disclosure may be used as a therapy for Gaucher disease (GD) (e.g., Type 1 GD, Type 2 GD, or Type 3 GD). In some embodiments, an AAV particle (e.g., an AAV particle comprising an AAV capsid variant), e.g., a plurality of particles, of the present disclosure may be used as a therapy for Parkinson's disease associated with a GBA mutation. In some embodiments, an AAV particle (e.g., an AAV particle comprising an AAV capsid variant), e.g., a plurality of particles, of the present disclosure may be used as a therapy for dementia with Lewy Bodies (DLB).

In some embodiments, an AAV particle (e.g., an AAV particle comprising an AAV capsid variant), e.g., a plurality of particles, of the present disclosure may be used as a therapy for spinal muscular atrophy.

In some embodiments, an AAV particle (e.g., an AAV particle comprising an AAV capsid variant), e.g., a plurality of particles, of the present disclosure may be used as a therapy for a leukodystrophy, e.g., Alexander disease, autosomal dominant leukodystrophy with autonomic diseases (ADLD), Canavan disease, cerebrotendinous xanthomatosis (CTX), metachromatic leukodystrophy (MLD), Pelizaeus-Merzbacher disease, or Refsum disease.

In some embodiments, an AAV particle (e.g., an AAV particle comprising an AAV capsid variant), e.g., a plurality of particles, of the present disclosure may be used as a therapy for chronic or neuropathic pain.

In some embodiments, an AAV particle (e.g., an AAV particle comprising an AAV capsid variant), e.g., a plurality of particles, of the present disclosure may be used as a therapy for a disease associated with expression of HER2, e.g., a disease associated with overexpression of HER2. In some embodiments, the AAV particle of the disclosure (e.g., an AAV particle comprising an AAV capsid variant) is useful for the treatment, prophylaxis, palliation or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, an AAV particle (e.g., an AAV particle comprising an AAV capsid variant) e.g., a plurality of particles, of the present disclosure may be used as a therapy for a neuro-oncological disorder. In some embodiments, the neuro-oncological disorder is a cancer of primary CNS origin (e.g., a cancer of a CNS cell and/or CNS tissue). In some embodiments, the neuro-oncological disorder is metastatic cancer in a CNS cell, CNS region, and/or a CNS tissue. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, an AAV particle (e.g., an AAV particle comprising an AAV capsid variant), e.g., a plurality of particles, of the present disclosure may be used as a therapy for a muscular disorder or a neuromuscular disorder.

In some embodiments, an AAV particle (e.g., an AAV particle comprising an AAV capsid variant), e.g., a plurality of particles, of the present disclosure may be used as a therapy for a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction.

In some embodiments, administration of the AAV particle described herein (e.g., an AAV particle comprising an AAV capsid polypeptide, e.g., an AAV capsid variant) to a subject may increase target gene, mRNA, and/or protein levels in a subject, relative to a control, e.g., the gene, mRNA, and/or mRNA levels in the subject prior to receiving AAV particle. The target gene, mRNA, and/or protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may increase the gene, mRNA, and/or protein levels of a target protein by fold increases over baseline. In some embodiments, AAV particles lead to 5-6 times higher levels of a target gene, mRNA, or protein.

In some embodiments, administration of the AAV particle described herein (e.g., an AAV particle comprising an AAV capsid polypeptide, e.g., an AAV capsid variant), e.g., an AAV particle comprising a nucleic acid encoding a siRNA molecule or an antibody or antibody fragment, to a subject may decrease target gene, mRNA, and/or protein levels in a subject, relative to a control, e.g., the gene, mRNA, and/or mRNA levels in the subject prior to receiving AAV particle. The target gene, mRNA, and/or protein levels may be decreased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may decrease the gene, mRNA, and/or protein levels of a target protein by fold decreases over baseline.

In some embodiments, the AAV particles of the present disclosure (e.g., an AAV particle comprising an AAV capsid polypeptide, e.g., an AAV capsid variant) may be used to increase target protein and reduce symptoms of neurological disease in a subject. In some embodiments, the AAV particles of the present disclosure (e.g., an AAV particle comprising an AAV capsid polypeptide, e.g., an AAV capsid variant) may be used to decrease target protein and reduce symptoms of neurological disease in a subject.

In some embodiments, the AAV particles of the present disclosure (e.g., an AAV particle comprising an AAV capsid polypeptide, e.g., an AAV capsid variant) may be used to reduce the decline of functional capacity and activities of daily living as measured by a standard evaluation system such as, but not limited to, the total functional capacity (TFC) scale.

In some embodiments, the AAV particles of the present disclosure (e.g., an AAV particle comprising an AAV capsid polypeptide, e.g., an AAV capsid variant) may be used to improve performance on any assessment used to measure symptoms of neurological disease. Such assessments include, but are not limited to ADAS-cog (Alzheimer Disease Assessment Scale-cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5 (6): 349-358 (2012)), the contents of which are herein incorporated by reference in their entirety.

In some embodiments, the present composition is administered as a solo therapeutic or as combination therapeutic for the treatment of a neurological disease/disorder or a neurodegenerative disorder, a muscular disorder or neuromuscular disorder, and/or a neuro-oncological disorder.

The AAV particles (e.g., an AAV particle comprising an AAV capsid variant) encoding the target protein may be used in combination with one or more other therapeutic agents. In some embodiments, compositions can be administered concurrently with, prior to, or subsequent to, additional therapeutic or medical procedures. In general, each agent will be administered at a dose and/or on a time schedule determined for that agent.

Therapeutic agents that may be used in combination with the AAV particles of the present disclosure (e.g., an AAV particle comprising an AAV capsid variant) can be small molecule compounds which are antioxidants, anti-inflammatory agents, anti-apoptosis agents, calcium regulators, anti-glutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles of the present disclosure (e.g., an AAV particle comprising an AAV capsid variant) for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle described herein (e.g., an AAV particle comprising an AAV capsid variant) may be co-administered with AAV particles expressing neurotrophic factors such as AAV-IGF-I (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In some embodiments, administration of the AAV particles (e.g., an AAV particle comprising an AAV capsid variant) to a subject will modulate, e.g., increase or decrease, the expression of a target protein in a subject and the modulation, e.g., increase or decrease of the presence, level, activity, and/or expression of the target protein will reduce the effects and/or symptoms of a neurological disease/disorder or a neurodegenerative disorder, a muscular disorder or neuromuscular disorder, and/or a neuro-oncological disorder in a subject.

### Definitions

Unless defined otherwise, all technical and scientific terms used herein have the same meaning as commonly understood by one of ordinary skill in the art to which the invention pertains.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” and “consisting essentially thereof” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to an AAV capsid, e.g., an AAV capsid variant, and a polynucleotide, e.g., a viral genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Amelioration: As used herein, the term “amelioration” or “ameliorating” refers to a lessening of severity of at least one indicator of a condition or disease. For example, in the context of a neurodegeneration disorder, amelioration includes the reduction of neuron loss.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of a gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. When referring to a measurable value such as an amount, a temporal duration, and the like, the term is meant to encompass is meant to encompass variations of ±20% or in some instances±10%, or in some instances±5%, or in some instances±1%, or in some instances±0.1% from the specified value, as such variations are appropriate to perform the disclosed methods.

Capsid: As used herein, the term “capsid” refers to the exterior, e.g., a protein shell, of a virus particle, e.g., an AAV particle, that is substantially (e.g., >50%, >90%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

### Complementary and Substantially Complementary: As Used Herein, the Term

“complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. The term “complementary” as used herein can encompass fully complementary, partially complementary, or substantially complementary. As used herein, the term “substantially complementary” means that the siRNA has a sequence (e.g., in the antisense strand) which is sufficient to bind the desired target mRNA, and to trigger the RNA silencing of the target mRNA. “Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95% or less. For example, gaps or discontinuities may be present in the capsid so long as the viral genome is retained in the capsid, e.g., prior to entry into a cell.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to production of an RNA template from a DNA sequence (e.g., by transcription). In some embodiments, expression further comprises one or more of: (1) processing of an RNA transcript (e.g., by splicing, editing, 5′ cap formation, and/or 3′ end processing); (2) translation of an RNA into a polypeptide or protein; and (3) post-translational modification of a polypeptide or protein.

Fragment: A “fragment,” as used herein, refers to a portion. For example, an antibody fragment may comprise a CDR, or a heavy chain variable region, or a scFv, etc. In some embodiments, a fragment is a nucleic acid fragment.

Identity: As used herein, “identity” refers to the subunit sequence identity between two polymeric molecules, e.g., between two nucleic acid molecules, such as, two DNA molecules or two

RNA molecules, or between two polypeptide molecules. When a subunit position in both of the two molecules is occupied by the same monomeric subunit; e.g., if a position in each of two DNA molecules is occupied by adenine, then they are identical at that position. The identity between two sequences is a direct function of the number of matching positions; e.g., if half (e.g., five positions in a polymer ten subunits in length) of the positions in two sequences are identical, the two sequences are 50% identical; if 90% of the positions (e.g., 9 of 10), are matched, the two sequences are 90% identical. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids

Research, 12 (1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Isolated: As used herein, the term “isolated” refers to a substance or entity that is altered or removed from the natural state, e.g., altered or removed from at least some of the components with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant or may be incorporated into a vector.

Neurological disease: As used herein, a “neurological disease” is any disease associated with the central or peripheral nervous system and components thereof (e.g., neurons).

Orthogonal evolution: As used herein, the term “orthogonal evolution” refers to a method wherein AAV particles are administered for a first round of AAV selection as described herein across a set of any number of cell- and/or subject-types that may be from different species and/or strains, and wherein any number of additional, e.g., subsequent, AAV selection rounds are performed either across a set of any number of cell- and/or subject-types that may be from different species and/or strains, or across a set of any number of cell- and/or subject-types that may be from the same species and/or strain.

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

RNAi agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

miR binding site: As used herein, a “miR binding site” comprises a nucleic acid sequence (whether RNA or DNA, e.g., differ by “U” of RNA or “T” in DNA) that is capable of binding, or binds, in whole or in part to a microRNA (miR), e.g., through complete or partial hybridization. Typically, such binding occurs between the miR and the miR binding site in the reverse complement orientation. In some embodiments, the miR binding site is transcribed from the AAV viral genome encoding the miR binding site.

In some embodiments, a miR binding site may be encoded or transcribed in series. Such a “miR binding site series” or “miR BSs” may include two or more miR binding sites having the same or different nucleic acid sequence.

Spacer: As used here, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

Sample: As used herein, the term “sample” or “biological sample” refers to a subset of its tissues, cells, nucleic acids, or component parts (e.g., body fluids, including but not limited to blood, serum, mucus, lymphatic fluid, synovial fluid, cerebrospinal fluid, saliva, amniotic fluid, amniotic cord blood, urine, vaginal fluid, and semen).

Self-complementary viral particle: As used herein, a “self-complementary viral particle” is a particle comprised of at least two components, a protein capsid and a self-complementary viral genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Target Cells: As used herein, “target cells” or “target tissue” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Variant: As used herein, the term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: As used herein, the term “functional variant” refers to a polypeptide variant or a polynucleotide variant that has at least one activity of the reference sequence.

Vector: As used herein, the term “vector” refers to any molecule or moiety which transports, transduces, or otherwise acts as a carrier of a heterologous molecule. In some embodiments, vectors may be plasmids. Vectors of the present disclosure may be produced recombinantly. The heterologous molecule may be a polynucleotide and/or a polypeptide.

Viral Genome: As used herein, the term “viral genome” refers to the nucleic acid sequence(s) encapsulated in an AAV particle. A viral genome comprises a nucleic acid sequence with at least one payload region encoding a payload and at least one ITR.

### Equivalents and Scope

The disclosures of each and every patent, patent application, and publication cited herein are hereby incorporated herein by reference in their entirety. Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1. HIGH-THROUGHPUT SCREEN OF TRACER AAV LIBRARY IN NHP AND MICE

A TRACER based method as described in WO 2020/072683, WO 2021/202651, and WO 2021/230987, the contents of which are herein incorporated by reference in their entirety, was used to generate the AAV capsid variants described herein. An orthogonal evolution approach was combined with high throughput screening by NGS. Briefly, various libraries of AAV capsid variants were generated using a sliding window approach using windows of multiple sizes in order to randomly introduce amino acid modifications, e.g., substitutions, into various positions across loop VIII of AAV5, including between residues 570-586, relative to a reference sequence numbered according to SEQ ID NO: 138. The initial library (passage 1) was passed first through cynomolgus macaques (Macaca fascicularis, n=2, 2-4 years of age) or human brain microvascular endothelial cells (hBMVECs) followed by monkey BMVECs. These first passage libraries were then pooled and screened through cynomolgus macaques (passage 2). After the second passage (e.g., 28 days post injection into two NHPs), RNA was extracted from multiple brain and spinal cord regions. Following RNA recovery and RT-PCR amplification, a systematic NGS enrichment analysis was performed to calculate fold enrichment relative to an AAV5 wild-type control. The top variants were selected for the creation of a synthetic library.

After creation of the synthetic library with the sub-selected variants, the synthetic library was screened (passage 3) in two NHPs (2-4 years of age) or mice (BALB/c or C57BL/6). The animals were injected intravenously with the synthetic library. After a period in vivo, (e.g., 28-days) RNA was extracted from nervous tissue, e.g., brain, spinal cord, and DRG of the NHPs. Following RNA recovery and RT-PCR amplification, a systematic NGS enrichment analysis was performed, and the peptides comprised within the variants were identified and the capsid enrichment ratio for each variant compared to the wild-type AAV5 control was calculated (fold enrichment relative to wild-type AAV5). Values above 1 indicate an increase in expression relative to AAV5. All NHPs were dosed intravenously at 2e13 VG/kg across the screen.

Following these three passages, approximately many variants were identified with an average fold change greater than wild-type AAV5 in the brain of the NHPs (Macaca fascicularis) and also mice. As shown in Table 9, the capsid variant comprising the amino acid sequence NAAQAY (SEQ ID NO: 943) resulted in a fold-change in expression of 110.8 in the brain of NHPs compared to the AAV5 control. This capsid variant also led to an increase in expression in the brain of both species of mice investigated, leading to a 141.6-fold increase in expression in the brain of BALB/c mice and a 16.9-fold increase in expression in the brain of C57BL/6 mice, compared to the AAV5 control.

TABLE 9
NGS fold-enrichment of AAV capsid variant in NHPs and mice

| | SEQ | Fold enrichment over AAV5 in | SEQ | Fold enrichment over | |
|---|---|---|---|---|---|
| Peptide | ID | the brain of NHPs (Macaca | ID | AAV5 in the brain of mice | |
| Sequence | NO: | fascicularis) | NO: | BALB/c | C57BL/6 |
| NAAQAY | 943 | 110.828 | 943 | 141.574 | 16.868 |

Taken together, these results demonstrate that after 3 rounds of screening of this AAV5 variant library with loop VIII modifications in NHPs (cynomolgus macaques (Macaca fascicularis)), and mice (BALB/c and C57BL6), many AAV capsid variants outperformed the wild-type AAV5, for example, in penetration of the blood brain barrier (BBB) and expression in the brain. Also, capsid variants were identified that could infect both mice and NHPs, indicating cross-species tropism and compatibility.

### Example 2. Evaluation of TTK-001 AAV Capsid Variant in Diverse Primate Species

This Example evaluates the tropism and cross-species compatibility of the TTK-001 (SEQ ID NO: 982 (amino acid) and 984 (DNA), comprising SEQ ID NO: 943) capsid variant in two diverse primate species, marmosets (Callithrix jacchus) and African green monkeys (Chlorocebus sabaeus), as compared to their tropism in cynomolgus macaques (Macaca fascicularis) provided in Example 1. The amino acid and DNA sequences of the TTK-001 capsid variant are provided, e.g., in Tables 4 and 5, respectively.

To investigate tropism in African green monkeys, AAV particles comprising the TTK-001 capsid variant or an AAV5 control under the control of a synapsin promoter, were intravenously injected into the African green monkeys (n=2, 3-12 years of age) at a dose of 2E13 vg/kg. After 14-days in life, the brains and tissues (liver, DRG, quadriceps, and heart) of the NHPs were collected and RNA was extracted. Following RNA recovery and RT-PCR amplification, a systematic NGS enrichment analysis was performed to calculate the fold enrichment ratio relative to the AAV5 wild-type control.

To investigate tropism in marmoset monkeys, AAV particles comprising the TTK-001 capsid variant, or an AAV5 control, were intravenously injected into marmosets (n=2, >10 months of age) at a dose of 2E13 vg/kg. After 28-days in life, the brains and tissues (liver quadriceps, and heart) of the NHPs were collected and RNA was extracted. Following RNA recovery and RT-PCR amplification, a systematic NGS enrichment analysis was performed to calculate the fold enrichment ratio relative to the AAV5 wild-type control.

As provided in Table 20 (African green monkeys) and Table 21 (marmosets), the TTK-001 capsid variant demonstrated increased CNS tropism in diverse primate species. The TTK-001 capsid variant demonstrated a 110.8-fold increase in expression relative to AAV5 in the brain of cynomolgus macaques (Table 9, Example 1), a 28.97-fold increase in expression relative to AAV5 in the brain of African green monkeys, and a 101.6-fold increase in expression relative to AAV5 in the brain of marmosets. Furthermore, TTK-001 also resulted in increased expression in the brain of

BALB/c mice and C57BL6 mice (Table 9, Example 1), demonstrating average fold changes in expression relative to AAV5 of 81.3 and 4.6, respectively.

TABLE 20
NGS-Fold Enrichment of TTK-001 in African green monkeys

| | SEQ ID | Fold Enrichment relative to AAV5 | | | | | |
|---|---|---|---|---|---|---|---|
| Sequence | NO: | Brain | DRG | Heart | Liver DNA | Liver RNA | Muscle |
| NAAQAY | 943 | 28.967 | 12.585 | 22.774 | 0.065 | 0.074 | 36.451 |

TABLE 21
NGS-Fold Enrichment of TTK-001 in Marmosets

| | | Fold Enrichment relative to AAV5 | | | | |
|---|---|---|---|---|---|---|
| | SEQ ID | | | Liver | Liver | |
| Sequence | NO: | Brain | Heart | DNA | RNA | Muscle |
| NAAQAY | 943 | 101.574 | 15.628 | 0.130 | 0.069 | 24.733 |

Taken together, these data demonstrate that the AAV5 capsid variant TTK-001 demonstrated increased CNS tropism relative to the AAV5 control in the CNS across three diverse primate species and rats, providing evidence of strong cross-species capacity.

### Example 3. Individual Capsid Characterization in NHPs

The goal of these experiments was to determine the transduction level, tropism, ability to cross the blood brain barrier, and overall spatial distribution in the central nervous system (CNS) and peripheral tissues of a capsid variant selected from the study described in Example 1 relative to AAV5 following intravenous injection in marmosets (Callithrix jacchus). The capsid variant was TTK-001 (SEQ ID NO: 982 (amino acid) and 984 (DNA), comprising SEQ ID NO: 943), as outlined in Table 3 above. The amino acid and DNA sequences of TTK-001 are provided, e.g., in Tables 4 and 5, respectively.

AAV particles were generated with the TTK-001 capsid variant, the AAV5 capsid control, or the AAV9 capsid control which comprised a self-complementary viral genome encoding a histone H2b protein with an S tag (TTK-001 capsid variant), T7 tag (AAV5 capsid control), or HA tag (AAV9 control capsid) driven by a ubiquitous CAG promoter. The AAV particles comprising the TTK-001 capsid variant, the AAV5 capsid control, or the AAV9 capsid control were administered to the marmosets (Callithrix jacchus) (n=3) intravenously in a single solution, at the doses indicated in Table 22. The in-life period was 28 days and then various CNS and peripheral tissues were collected for measuring transgene mRNA (expression) by RT-qPCR, protein expression by IHC, and viral DNA (biodistribution) by ddPCR. Data were then normalized to the dose of each viral vector in the dosing solution.

TABLE 22
Titer of the AAV particles comprising the various
capsids in solution dosed in marmosets

| | | Ratio of Capsid Variant to |
|---|---|---|
| Capsid Variant | Actual Titer Dosed | AAV9 |
| AAV5 | 2.88 vg/mL | 0.72 |
| AAV9 | 4.00 vg/mL | 1.0 |
| TTK-001 | 2.69 vg/mL | 0.67 |

Biodistribution and transgene expression of TTK-001 was measured in the peripheral tissues, including the muscle (quadriceps), heart, and liver, of the marmosets. As shown in Table 23 and Table 24, the TTK-001 capsid variant, an AAV5 capsid variant, demonstrated increased biodistribution and transgene expression in the muscle (quadriceps) relative to the AAV5 and the AAV9 control capsids. More specifically, TTK-001 demonstrated 11.99 fold-greater transgene expression relative to AAV9 and 21.34 fold-greater transgene expression relative to AAV5 in the muscle of marmosets. In the heart, the TTK-001 capsid variant demonstrated increased biodistribution (Table 23) and transgene expression (Table 24) relative to AAV5, and increased biodistribution (Table 23) and comparable transgene expression (Table 24) relative to AAV9. In the liver, the TTK-001 capsid variant exhibited lower biodistribution (Table 23) and transgene expression (Table 24) relative to both AAV5 and AAV9, indicating that the TTK-001 capsid variant was detargeted in the liver relative to AAV5 and AAV9 in marmosets.

Biodistribution and transgene expression of TTK-001 was also measured in various tissues of the central nervous system (Table 23 and Table 24). TTK-001 also demonstrated increased biodistribution and transgene expression in the caudate and motor cortex in the brain of the marmosets relative to the AAV5 capsid control, as well as the AAV9 capsid control (Tables 23 and 24). Similar expression and distribution was observed by immunohistochemistry. More specifically, staining for TTK-001 was detected in the caudate, putamen, thalamus, and cerebellum, and this staining was increased relative to AAV5. Staining for TTK-001 was also observed in the molecular and granule layer of the cerebellum.

TABLE 23
Quantification of viral genome copies per diploid genome
(biodistribution) by ddPCR following intravenous administration
of AAV particles comprising a TTK-001 capsid normalized
to the actual titer of the viral vector in the dosing solution
(vg/dg = viral genome copies/diploid genome)

| | Tissue | | | | | |
|---|---|---|---|---|---|---|
| | Caudate | | | Motor Cortex | | |
| | | vg/dg | vg/dg | | vg/dg | vg/dg |
| | | relative to | relative to | | relative to | relative to |
| Capsid | vg/dg | AAV9 | AAV5 | vg/dg | AAV9 | AAV5 |
| AAV9 | 0.01 | 1.00 | — | 0.02 | 1.00 | — |
| AAV5 | 0.03 | 3.12 | 1.00 | 0.05 | 2.10 | 1.0 |
| TTK-001 | 0.33 | 29.53 | 9.44 | 0.54 | 24.75 | 11.65 |
| | Heart | | | Muscle | | |
| | | vg/dg | vg/dg | | vg/dg | vg/dg |
| | | relative to | relative to | | relative to | relative to |
| Capsid | vg/dg | AAV9 | AAV5 | vg/dg | AAV9 | AAV5 |
| AAV9 | 0.48 | 1.00 | — | 0.23 | 1.00 | — |
| AAV5 | 0.40 | 0.80 | 1.00 | 1.18 | 6.08 | 1.00 |
| TTK-001 | 3.63 | 6.97 | 8.47 | 3.02 | 13.85 | 2.52 |

| | Liver | | |
|---|---|---|---|
| | | vg/dg | vg/dg |
| | | relative to | relative to |
| Capsid | vg/dg | AAV9 | AAV5 |
| AAV9 | 13.79 | 1.00 | — |
| AAV5 | 18.28 | 1.41 | 1.00 |
| TTK-001 | 3.90 | 0.29 | 0.22 |

TABLE 24
Quantification of transgene mRNA by RT-qPCR following intravenous
administration of AAV particles comprising a TTK-001 capsid normalized
to the actual titer of the viral vector in the dosing solution (mRNA =
transgene mRNA fold over housekeeping gene; rel. to AAV9 =
transgene mRNA fold over housekeeping gene relative to AAV9; rel. to
AAV5 transgene mRNA fold over housekeeping gene relative to AAV5)

| | Tissue | | | | | |
|---|---|---|---|---|---|---|
| | Caudate | | | Motor Cortex | | |
| Capsid | mRNA | rel. AAV9 | rel. AAV5 | mRNA | rel. AAV9 | rel. AAV5 |
| AAV9 | 0.03 | 1.00 | — | 0.05 | 1.00 | — |
| AAV5 | 0.02 | 0.61 | 1.00 | 0.01 | 0.31 | 1.00 |
| TTK-001 | 0.15 | 5.25 | 8.56 | 0.17 | 3.61 | 13.34 |
| | Heart | | | Muscle | | |
| Capsid | mRNA | rel. AAV9 | rel. AAV5 | mRNA | rel. AAV9 | rel. AAV5 |
| AAV9 | 12.67 | 1.00 | — | 0.15 | 1.00 | — |
| AAV5 | 4.36 | 0.30 | 1.0 | 0.11 | 0.48 | 1.00 |
| TTK-001 | 15.97 | 1.19 | 4.07 | 3.27 | 11.99 | 21.34 |

| | Liver | | |
|---|---|---|---|
| Capsid | mRNA | rel. AAV9 | rel. AAV5 |
| AAV9 | 1.85 | 1.00 | — |
| AAV5 | 6.89 | 3.35 | 1.0 |
| TTK-001 | 0.57 | 0.29 | 0.09 |

### CONCLUSIONS

Taken together, these data demonstrate that TTK-001, which is an AAV5 capsid variant, with enhanced muscle tropism as well as enhanced tropism in the central nervous system, following intravenous injection in marmosets.

We claim:

1. An AAV5 capsid variant comprising the amino acid sequence of positions 193-724 of SEQ ID NO: 982, or an amino acid sequence at least 95% identical thereto, wherein the AAV capsid variant comprises an N at position 578 and an A at position 580, numbered according to SEQ ID NO: 982.

2. An AAV5 capsid variant comprising the amino acid sequence of positions 137-724 of SEQ ID NO: 982, or an amino acid sequence at least 95% identical thereto, wherein the AAV capsid variant comprises an N at position 578 and an A at position 580, numbered according to SEQ ID NO: 982.

3. An AAV5 capsid variant comprising the amino acid sequence of SEQ ID NO: 982, or an amino acid sequence at least 95% identical thereto, wherein the AAV capsid variant comprises an N at position 578 and an A at position 580, numbered according to SEQ ID NO: 982.

4. The AAV5 capsid variant of any one of claims 1-3, which further comprises one, two, or all of an amino acid other than A at position 581, T at position 582, and/or G at position 583, numbered according to SEQ ID NO: 138.

5. The AAV capsid variant of any one of claims 1-4, which further comprises one, two, or all of Q at position 581, A at position 582, and Y at position 583, numbered according to SEQ ID NO: 982.

6. An AAV5 capsid variant comprising the amino acid sequence of positions 193-724 of SEQ ID NO: 982, or an amino acid sequence at least 95% identical thereto, wherein the AAV capsid variant comprises an N at position 578, A at position 580, Q at position 581, A at position 582, and Y at position 583, numbered according to SEQ ID NO: 982.

7. An AAV5 capsid variant comprising the amino acid sequence of positions 137-724 of SEQ ID NO: 982, or an amino acid sequence at least 95% identical thereto, wherein the AAV capsid variant comprises an N at position 578, A at position 580, Q at position 581, A at position 582, and Y at position 583, numbered according to SEQ ID NO: 982.

8. An AAV5 capsid variant comprising the amino acid sequence of SEQ ID NO: 982, or an amino acid sequence at least 95% identical thereto, wherein the AAV capsid variant comprises an N at position 578, A at position 580, Q at position 581, A at position 582, and Y at position 583, numbered according to SEQ ID NO: 982.

9. The AAV5 capsid variant of any one of claim 1, or 4-6, which comprises the amino acid sequence of positions 193-724 of SEQ ID NO: 982.

10. The AAV5 capsid variant of any one of claim 1, 2, or 4-7, which comprises the amino acid sequence of positions 137-724 of SEQ ID NO: 982.

11. The AAV5 capsid variant of any one of claims 1-10, which comprises the amino acid sequence of SEQ ID NO: 982.

12. The AAV5 capsid variant of any one of claims 1-11, wherein the nucleotide sequence encoding the AAV5 capsid variant comprises SEQ ID NO: 984, or a nucleotide sequence at least 95% identical thereto.

13. The AAV5 capsid variant of any one of claims 1-12, wherein the AAV5 capsid variant has one, two, three, four, or all of the following properties:
(i) has an increased tropism for a muscle cell or tissue, relative to the tropism of a reference sequence comprising the amino acid sequence of SEQ ID NO: 138 or SEQ ID NO: 139;
(ii) transduces a muscle region, optionally wherein the level of transduction is at least 2, 5, 10, 15, 20, or 25-fold greater as compared to a reference sequence of SEQ ID NO: 138 or 139, e.g., when measured by an assay, e.g., an immunohistochemistry assay or a qPCR assay, e.g., as described in Example 3;
(iii) delivers an increased level of a payload to a muscle cell or region, optionally wherein the level of the payload is increased by at least 5, 10, 12, 15, 20, 21, or 25-fold, as compared to a reference sequence of SEQ ID NO: 138 or SEQ ID NO: 139, e.g., when measured by an assay, e.g., a qRT-PCR or a qPCR assay (e.g., as described in Example 3;
(iv) delivers an increased level of viral genomes to a muscle cell or region, optionally wherein the level of viral genomes is increased by at least 2, 2.5, 5, 5.5, 6, 6.5, 7, 7.5, 8, 8.5, 9, 9.5, 10, 10.5, 11, 11.5, 12, 12.5, 13, 13.5, 13.8, or 14-fold, as compared to a reference sequence of SEQ ID NO: 138 or SEQ ID NO: 139, e.g., when measured by an assay, e.g., a qRT-PCR or a qPCR assay (e.g., as described in Example 3); and/or
(v) has an decreased tropism for a liver cell or tissue, relative to the tropism of a reference sequence comprising the amino acid sequence of SEQ ID NO: 138 or SEQ ID SEQ ID NO: 139

14. The AAV5 capsid variant of any one of claims 1-13, wherein the AAV5 capsid variant has one, two, three, four, or all of the following properties:
(i) has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138 or SEQ ID SEQ ID NO: 139;
(ii) transduces a brain cell or region, e.g., (e.g., the caudate, motor cortex, putamen, thalamus, and/or cerebellum (e.g., the molecular and granule layer of the cerebellum)), optionally wherein the level of transduction is at least 3, 4, 5, 6, 7, 8, 9, 10, 11, 12, 13, 14, 15, 20, 24, 25, 29, or 30-fold greater as compared to a reference sequence of SEQ ID NO: 138 or SEQ ID NO: 139, e.g., when measured by an assay, e.g., an immunohistochemistry assay or a qPCR assay, e.g., as described in Example 3;
(iii) is enriched at least about 4, 4.6, 10, 20, 25, 28, 30, 40, 50, 60, 70, 80, 81, 80, 100, 101, or 110-fold, in the brain compared to a reference sequence of SEQ ID NO: 138, e.g., when measured by an assay as described in Example 1 or 2;
(iv) delivers an increased level of a payload to a brain cell or region, optionally wherein the level of the payload is increased by at least 3, 4, 5, 6, 7, 8, 9, 10, 11, 12, 13, or 14-fold, as compared to a reference sequence of SEQ ID NO: 138 or SEQ ID NO: 139, e.g., when measured by an assay, e.g., a qRT-PCR or a qPCR assay (e.g., as described in Example 3), optionally wherein the brain region is a caudate, motor cortex, putamen, thalamus, and/or cerebellum (e.g., the molecular and granule layer of the cerebellum); and/or
(v) delivers an increased level of viral genomes to a brain cell or region, optionally wherein the level of viral genomes is increased by at least 3, 4, 5, 6, 7, 8, 9, 10, 11, 12, 13, 14, 15, 20, 24, 25, 29, or 30-fold, as compared to a reference sequence of SEQ ID NO: 138 or SEQ ID NO: 139, e.g., when measured by an assay, e.g., a qRT-PCR or a qPCR assay (e.g., as described in Example 3), optionally wherein the brain region is a caudate, motor cortex, putamen, thalamus, and/or cerebellum (e.g., the molecular and granule layer of the cerebellum).

15. A polynucleotide encoding the AAV5 capsid variant of any one of claims 1-14.

16. The polynucleotide of claim 15, which comprises the nucleotide sequence of SEQ ID NO: 984, or a nucleotide sequence at least 95% identical thereto.

17. A peptide comprising:
(a) the amino acid sequence of SEQ ID NO: 943;
(b) an amino acid sequence comprising at least 4 or 5 consecutive amino acids from SEQ ID NO: 943;
(c) an amino acid sequence comprising one, two, or three but no more than four different amino acids, relative to the amino acid sequence of SEQ ID NO: 943; or
(d) an amino sequence comprising one, two, or three but no more than four modifications, e.g., substitutions (e.g., conservative substitutions), insertions, or deletions, relative to the amino acid sequence of SEQ ID NO: 943.

18. An AAV5 capsid variant comprising the peptide of claim 17.

19. An AAV particle comprising the AAV capsid variant of any one of claim 1-14 or 18, an AAV capsid variant encoded by the polynucleotide of claim 15 or 16, or an AAV capsid variant comprising the peptide of claim 17.

20. The AAV particle of claim 19, which comprises a nucleotide sequence encoding a payload, optionally wherein the encoded payload comprises a therapeutic protein or functional variant thereof;
an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.

21. The AAV particle of claim 20, wherein:
(i) the therapeutic protein or functional variant thereof, e.g., a recombinant protein, is associated with (e.g., aberrantly expressed in) a neurological or neurodegenerative disorder, a muscular, a muscular dystrophy, neuromuscular disorder, or a neuro-oncological disorder, optionally wherein the therapeutic protein or functional variant thereof is chosen from apolipoprotein E (APOE) (e.g., ApoE2, ApoE3 and/or ApoE4); human survival of motor neuron (SMN) 1 or SMN2;
glucocerebrosidase (GBA1); aromatic L-amino acid decarboxylase (AADC); aspartoacylase (ASPA);
tripeptidyl peptidase I (CLN2); beta-galactosidase (GLB1); N-sulphoglucosamine sulphohydrolase (SGSH); N-acetyl-alpha-glucosaminidase (NAGLU); iduronate 2-sulfatase (IDS); intracellular cholesterol transporter (NPC1); gigaxonin (GAN); titn; myotubularin; calpain-3 (CAPN-3); dysferlin (DYSF); gamma-sarcoglycan (SGCG); alpha-sarcoglycan (SGCA); microdystrophin; dystrophin;
beta-sarcoglycan (SGCB); fukutin-related protein (FKRP); anoctamin-5 (ANO5); or a combination thereof;
(ii) the antibody or antibody binding fragment binds to
(a) a CNS related target, e.g. an antigen associated with a neurological or neurodegenerative disorder, e.g., β-amyloid, APOE, tau, SOD1, TDP-43, huntingtin (HTT), and/or synuclein;
(b) a muscular or neuromuscular related target, e.g., an antigen associated with a muscular or neuromuscular disorder; or
(c) a neuro-oncology related target, e.g., an antigen associated with a neuro-oncological disorder, e.g., HER2, or EGFR (e.g., EGFRvIII);
(iii) the enzyme comprises a meganuclease, a zinc finger nuclease, a TALEN, a recombinase, integrase, a base editor, a Cas9, or a fragment thereof;
(iv) the component of a gene editing system comprises one or more components of a CRISPR-Cas system, optionally wherein the one or more components of the CRISPR-Cas system comprises a Cas9, e.g., a Cas9 ortholog or a Cpf1, and a single guide RNA (sgRNA), wherein:
(a) the sgRNA is located upstream (5′) of the cas9 enzyme; and/or
(b) the sgRNA is located downstream (3′) of the cas9 enzyme; and/or
(v) the RNAi agent (e.g., a dsRNA, siRNA, shRNA, pre-miRNA, pri-miRNA, miRNA, stRNA, lncRNA, piRNA, or snoRNA), modulates, e.g., inhibits, expression of, a CNS related gene, mRNA, and/or protein, optionally wherein the CNS related gene is chosen from SOD1, MAPT, APOE, HTT, C9ORF72, TDP-43, APP, BACE, SNCA, ATXN1, ATXN3, ATXN7, SCNIA-SCN5A, SCN8A-SCN11A, or a combination thereof.

22. The AAV particle of any one of claims 19-21, which comprises a viral genome comprising a promoter operably linked to the nucleic acid sequence encoding the payload, optionally wherein:
(i) the promoter is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor β-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light chain (NFL) or heavy chain (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., aMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof;
(ii) the promoter is an EF-1a promoter variant, e.g., a truncated EF-1a promoter; or
(iii) the promoter comprises the nucleotide sequence of any one of SEQ ID NOs: 987-1007, a nucleotide sequence comprising at least one, two, three, four, five, six, or seven but no more than four modifications, e.g., substitutions, relative to the nucleotide sequence of SEQ ID NOs: 987-1007, or a nucleotide sequence with at least 80% (e.g., 85%, 90%, 95%, 96%, 97%, 98%, or 99%) sequence identity to any one of SEQ ID NOs: 987-1007.

23. The AAV particle of claim 22, wherein the viral genome further comprises:
(i) a polyA signal sequence;
(ii) an inverted terminal repeat (ITR) sequence, optionally wherein the ITR sequence is positioned 5′ relative to the encoded payload and/or the ITR sequence is positioned 3′ relative to the encoded payload;
(iii) an enhancer, a Kozak sequence, an intron region, and/or an exon region; and/or
(iv) a nucleotide sequence encoding a miR binding site, e.g., a miR binding site that modulates, e.g., reduces, expression of the antibody molecule encoded by the viral genome in a cell or tissue where the corresponding miRNA is expressed, optionally wherein the encoded miR binding site modulates, e.g., reduces, expression of the encoded antibody molecule in a cell or tissue of the DRG, liver, heart, hematopoietic lineage, or a combination thereof.

24. The AAV particle of claim 22 or 23, wherein the viral genome comprises:
(i) at least 1-5 copies of the encoded miR binding site, e.g., at least 1, 2, 3, 4, or 5 copies;
(ii) at least 3 copies of an encoded miR binding sites, optionally wherein:
(a) all three copies comprise the same miR binding site, or at least one, two, three, or all of the copies comprise a different miR binding site; and/or
(b) the 3 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA; or
(iii) at least 4 copies of an encoded miR binding site, optionally wherein
(a) all four copies comprise the same miR binding site, or at least one, two, three, or all of the copies comprise a different miR binding site; and/or
(b) the 4 copies of the encoded miR binding sites are continuous (e.g., not separated by a spacer), or are separated by a spacer, optionally wherein the spacer comprises the nucleotide sequence of GATAGTTA, or a nucleotide sequence having at least one, two, or three modifications, e.g., substitutions (e.g., conservative substitutions), but no more than four modifications, e.g., substitutions (e.g., conservative substitutions), relative to GATAGTTA.

25. The AAV particle of claim 23 or 24, wherein the encoded miR binding site comprises a miR122 binding site, a miR183 binding site, a miR-1 binding site, a miR-142-3p, or a combination thereof, optionally wherein:
(i) the encoded miR122 binding site comprises the nucleotide sequence of SEQ ID NO: 4673, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4673;
(ii) the encoded miR183 binding site comprises the nucleotide sequence of SEQ ID NO: 4676, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4676;
(iii) the encoded miR-1 binding site comprises the nucleotide sequence of SEQ ID NO: 4679, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4679; and/or
(iv) the encoded miR-142-3p binding site comprises the nucleotide sequence of SEQ ID NO: 4675, or a nucleotide sequence substantially identical (e.g., having at least 70%, 75%, 80%, 85%, 90%, 92%, 95%, 97%, 98%, or 99% sequence identity) thereto; or a nucleotide sequence comprising at least one, two, three, four, five, six, or seven modifications, e.g., substitutions, insertions, or deletions, but no more than ten modifications, e.g., substitutions, insertions, or deletions, relative to SEQ ID NO: 4675.

26. The AAV particle of any one of claims 19-25, wherein the viral genome is:
(i) single stranded; or
(ii) self-complementary

27. The AAV5 capsid variant, polynucleotide, peptide, or AAV particle of any one of the preceding claims which is isolated, e.g., recombinant.

28. A vector comprising a polynucleotide encoding the AAV5 capsid variant of any one of claim 1-14, 18, or 27, the polynucleotide of any one of claim 15, 16, or 27, or a polynucleotide encoding the peptide of claim 17 or 27.

29. A cell, e.g., a host cell, comprising the AAV5 capsid variant of any one of claim 1-14, 18, or 27, the polynucleotide of any one of claim 15, 16, or 27, a polynucleotide encoding the peptide of claim 17 or 27, the AAV particle of any one of claims 19-27, or the vector of claim 28, optionally wherein:
(i) the cell is a mammalian cell or an insect cell;
(ii) the cell is a cell of a brain region or a spinal cord region, optionally a cell of the caudate, motor cortex, putamen, thalamus, or cerebellum (e.g., the molecular and granule layer of the cerebellum); and/or
(iii) the cell is a neuron, a sensory neuron, a motor neuron, an astrocyte, a glial cell, oligodendrocyte, or a muscle cell (e.g., a cell of the heart, diaphragm, or quadriceps).

30. A method of making an AAV particle, comprising
(i) providing a host cell comprising a viral genome; and
(ii) incubating the host cell under conditions suitable to enclose the viral genome in the AAV5 capsid variant of any one of claim 1-14, 18, or 27, or an AAV capsid variant encoded by the polynucleotide of any one of claim 15, 16, or 27;
thereby making the AAV particle.

31. A pharmaceutical composition comprising the AAV particle of any one of claims 19-27, an AAV particle comprising the AAV capsid variant of any one of claim 1-14, 18, or 27, or an AAV particle comprising the peptide of claim 17 or 27, and a pharmaceutically acceptable excipient.

32. A method of delivering a payload to a cell or tissue (e.g., a CNS cell or a CNS tissue; or a muscle cell or tissue), comprising administering an effective amount of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27.

33. The method of claim 32, wherein the cell is:
(i) a cell of a brain region or a or a spinal cord region, optionally a cell of the t caudate, motor cortex, putamen, thalamus, cerebellum (e.g., the molecular and granule layer of the cerebellum), or a combination thereof;
(ii) is a cell of the heart, e.g., a heart atrium or a heart ventricle;
(iii) is a cell of the muscle (e.g., a cell of the quadriceps);
(iv) a neuron, a sensory neuron, a motor neuron, an astrocyte, a glial cell, or an oligodendrocyte;
(v) within a subject, optionally wherein the subject has, has been diagnosed with having, or is at risk of having a genetic disorder (e.g., a monogenic disorder or a polygenic disorder), a neurological disorder (e.g., a neurodegenerative disorder), a neuro-oncological disorder, a muscular disorder, a muscular dystrophy, or a neuromuscular disorder.

34. A method of treating a subject having or diagnosed with having a genetic disorder, e.g., a monogenic disorder or a polygenic disorder, comprising administering to the subject an effective amount of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27.

35. A method of treating a subject having or diagnosed with having a neurological disorder, e.g., a neurodegenerative disorder, comprising administering to the subject an effective amount of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27.

36. A method of treating a subject having or diagnosed with having a muscular disorder, a muscular dystrophy, or a neuromuscular disorder, comprising administering to the subject an effective amount of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27.

37. A method of treating a subject having or diagnosed with having a neuro-oncological disorder, comprising administering to the subject an effective amount of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27.

38. The method of any one of claims 33-37, wherein the genetic disorder, neurological disorder, neurodegenerative disorder, muscular disorder, muscular dystrophy, neuromuscular disorder, or neuro-oncological disorder is Duchenne muscular dystrophy (DMD), Limb-Girdle Muscular Dystrophy (LGMD2A), Becker muscular dystrophy (BMD), congenital muscular dystrophy, Facioscapulohumeral muscular dystrophy, titinopathy, Emery Dreifuss muscular dystrophy, X-linked myotubular myopathy, Huntington's Disease, Amyotrophic Lateral Sclerosis (ALS), Gaucher Disease, Dementia with Lewy Bodies, Parkinson's disease, Spinal Muscular Atrophy, Alzheimer's Disease, a leukodystrophy (e.g., Alexander disease, autosomal dominant leukodystrophy with autonomic diseases (ADLD)), Canavan disease, cerebrotendinous xanthomatosis (CTX), metachromatic leukodystrophy (MLD), Pelizaeus-Merzbacher disease, Refsum disease, or a cancer (e.g., a HER2/neu positive cancer or a glioblastoma).

39. The method of any one of claims 34-38, wherein treating comprises prevention of progression of the disease or disorder in the subject.

40. The method of any one of claims 33-39, wherein the subject is a human.

41. The method of any one of claims 32-40, wherein the AAV particle is administered to the subject:
(i) intravenously, via intra-cisterna magna injection (ICM), intracerebrally, intrathecally, intracerebroventricularly, via intraparenchymal administration, or intramuscularly;
(ii) via focused ultrasound (FUS), e.g., coupled with the intravenous administration of microbubbles (FUS-MB), or MRI-guided FUS coupled with intravenous administration;
(iii) intravenously; or
(iv) intramuscularly.

42. The pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27, for use in a method of delivering a payload to a cell or tissue.

43. The pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27, for use in a method of treating a genetic disorder, a neurological disorder, a neurodegenerative disorder, a muscular disorder, a muscular dystrophy, a neuromuscular disorder, or a neuro-oncological disorder.

44. The pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27, for use in the manufacture of a medicament.

45. Use of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27, in the manufacture of a medicament.

46. Use of the pharmaceutical composition of claim 31, the AAV particle of any one of claims 19-27, an AAV particle comprising the AAV capsid variant of any one of claim 1-14, 18, or 27, or an AAV particle comprising the peptide of claim 17 or 27, in the manufacture of a medicament for treating a genetic disorder, a neurological disorder, a neurodegenerative disorder, a muscular disorder, a muscular dystrophy, a neuromuscular disorder, or a neuro-oncological disorder.
